(19) United States
(12) Patent Application Publication
(10) Pub. No.: US 2024/0349766 A1
(43) Pub. Date: Oct. 24, 2024

(54) COMPOSITIONS FOR PREPARING ANIMAL-FREE EGG-LIKE PRODUCTS

(71) Applicant: Clara Foods Co., Daly City, CA (US)

(72) Inventors: Kritika MAHADEVAN, Daly City, CA (US); Ying Joy ZHONG, Fremont, CA (US); Isha JOSHI, Daly City, CA (US); Farnoosh AYOUGHI, Daly City, CA (US); Ranjan PATNAIK, Daly City, CA (US); A Samuel POTTINGER, Daly City, CA (US); Dane Mathias JACOBSON, Newton, MA (US); Andrew Keum Min MIYASHIRO, Foster City, CA (US); Harshal KSHIRSAGAR, Daly City, CA (US)

(73) Assignee: Clara Foods Co., Daly City, CA (US)

(21) Appl. No.: 18/761,860

(22) Filed: Jul. 2, 2024

Related U.S. Application Data
(63) Continuation of application No. 18/057,134, filed on Nov. 18, 2022.
Application No. 18/057,134 is a continuation of application No. PCT/US2022/017580, filed on Feb. 23, 2022.
(60) Provisional application No. 63/152,489, filed on Feb. 23, 2021.

(51) Int. Cl. A23L 15/00; A23J 1/14; A23J 1/18; C07K 14/465; C07K 14/77

(52) U.S. Cl. CPC A23L 15/35; A23J 1/14; A23J 1/18; C07K 14/465; C07K 14/77; A23V 2002/00

(57) ABSTRACT

The present disclosure provides compositions for preparation of egg-like products.

### CROSS-REFERENCE TO RELATED APPLICATIONS

This application is a continuation of U.S. patent application Ser. No. 18/057,134, filed Nov. 18, 2022, which is a continuation of International Patent Application No. PCT/US2022/017580, filed Feb. 23, 2022, which claims priority to U.S. Provisional Patent Application No. 63/152,489, filed Feb. 23, 2021; each of which is incorporated by reference herein in its entirety.

### SEQUENCE LISTING

The instant application contains a Sequence Listing which has been submitted in XML format and is hereby incorporated by reference in its entirety. Said XML copy, created on Nov. 18, 2022, is named 49160-720.301.xml and is 207,858 bytes in size.

### BACKGROUND

Alternatives to eggs have been researched in an attempt to find egg substitutes for subjects who would like to or are required to avoid eggs. More recently, egg alternatives have been used in different aspects of cooking, such as in baking. Yet, when used alone or as an ingredient to create a food product many egg alternatives differ substantially from eggs in taste, color, cohesiveness, and texture. Thus, there is a need for an improved egg substitute to be used alone or as in ingredient in a food product.

### SUMMARY OF THE INVENTION

The present disclosure is in the field of food products and specifically relates to a liquid composition used in the preparation of, or replacement of, an egg-like product. In particular, the egg substitute may be used in or as food items, such as a scramble. The egg substituted may be formed from one or more recombinantly-produced proteins, e.g., recombinantly-produced ovomucoid (rOVD) and recombinantly-produced ovalbumin (rOVA).

Certain embodiments provided herein include a composition comprising a substantially liquid mixture for preparation of, or replacement of, an egg-like product, the substantially liquid mixture may comprise: (a) one or more egg-related proteins selected from the group consisting of a recombinant ovomucoid (rOVD), and a recombinant ovalbumin (rOVA), and a recombinant lysozyme (rOVL); (b) a protein component, wherein the protein component may comprise a plant protein; (c) a dietary fiber-providing component, wherein the dietary fiber-providing component may comprise a plant fiber; (d) a starch-providing component, wherein the starch-providing component may comprise polysaccharides, e.g., having glucose monomers joined via α-1,4 linkages; (e) a gelation agent; (f) a salt and/or another flavoring agent; (g) a lipid component; and (h) water. In some embodiments, the liquid mixture may be substantially devoid of any hen-derived egg protein. In some embodiments, the composition may be a vegan composition. In some embodiments, the one or more egg-related proteins may comprise rOVD.

In some aspects, described herein are liquid whole egg substitute compositions. In some embodiments, the composition may comprise: (a) recombinant egg-white proteins may consist of a recombinant ovomucoid (rOVD) and a recombinant ovalbumin (rOVA); (b) one or more gelation/thickening agents; (c) a salt and/or another flavoring agent; (d) a lipid component; and (e) water; wherein a weight ratio of recombinant egg-white proteins to lipid component may be greater than 1:1.

In some embodiments, the weight ratio of rOVD and rOVA may be from about 1:50 to about 2:1. In some embodiments, the weight percent of protein to composition may be greater than about 2% on a w/w basis. In some embodiments, the weight percent of protein to composition may be less than about 15% on a w/w basis. In some embodiments, the composition lacks any animal-derived substances or any animal-derived components.

In some embodiments, a weight ratio of rOVD and rOVA may be less than about 1:50, may be less than about 1:40, may be less than about 1:30, may be less than about 1:20, may be less than about 1:10, may be less than about 1:5, may be less than about 1:4, may be less than about 1:3, may be less than about 1:2, less than about 1:1, or may be less than about 2:1. In some embodiments, the weight percent of rOVA to composition may be from about 2% to about 10% on a w/w basis. In some embodiments, the rOVA has one or more N-linked glycosylation sites having mannose linked to an N-acetyl glucosamine, and wherein the N-linked glycosylation sites lack galactose.

In some embodiments, the rOVA has at least 80%, 85%, 90%, 92%, 95%, 96%, 97%, 98%, 99% of 100% sequence identity to any one of SEQ ID NO: 45 to SEQ ID NO: 118. In some embodiments, the weight percent of rOVD to composition may be from about 0.15% to about 4.5% on a w/w basis. In some embodiments, the rOVD comprises a glycosylation pattern that differs from the glycosylation pattern of a native chicken ovomucoid. In some embodiments, the rOVD comprises at least one glycosylated asparagine residue. In some embodiments, the rOVD may be substantially devoid of N-linked mannosylation. In some embodiments, the rOVD has at least 80%, 85%, 90%, 92%, 95%, 96%, 97%, 98%, 99% of 100% sequence identity to any one of SEQ ID NO. 1 to SEQ ID NO: 44.

In some embodiments, when the composition and a whole hen's egg are prepared as a scramble, the scrambled composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the composition and a composition may comprise a protein component may consist of proteins obtained from a plant are prepared as a scramble, the scrambled composition provides better sensory attributes than those of a scrambled composition may comprise a protein component may consist of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, the amino acid profile of the recombinant egg-white proteins may be closer to a whole hen's egg than the amino acid profile of a protein component may consist of proteins obtained from a plant. The amino acid profile may be calculated as the percent by mass of each amino acid in the protein source. For instance, mass of cysteine in whole hens egg divided by mass of protein in whole hens egg. In some embodiments, the nutrition value provided by amino acids of the recombinant egg-white proteins may be closer to a whole hen's egg than the nutrition value provided by amino acids of a protein component may consist of proteins obtained from a plant. In some embodiments, the recombinant egg-white protein comprises a fraction of cysteine, methionine, and/or lysine amino acids that may be closer to the fraction in a whole hen's egg than the fraction in a protein component may consist of proteins obtained from a plant.

In some embodiments, the recombinant egg-white protein comprises a larger fraction of cysteine, methionine, and/or lysine amino acids than the fraction in a composition may comprise a protein component may consist of proteins obtained from a plant. In some embodiments, the recombinant egg-white protein comprises a fraction of cysteine and methionine amino acids closer to the fraction in a whole hen's egg than the fraction in a protein component may consist of proteins obtained from a plant. In some embodiments, the fraction of cysteine, methionine, and/or lysine amino acids in the recombinant egg-white proteins provides, in part, a flavor and/or smell that approximates the flavor and/or smell of a whole hen's egg.

In some embodiments, the fraction of cysteine, methionine, and/or lysine amino acids in the recombinant egg-white proteins provides, in part, a flavor and/or smell that may be superior to the flavor and/or smell of composition may comprise a protein component may consist of proteins obtained from a plant. In some embodiments, the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein. In some embodiments, the proteins obtained from a plant comprise or consist of chickpea protein and mung bean protein or the proteins obtained from a plant comprise or consist of lupin bean protein and pea protein.

In some aspects, provided herein is a powdered whole egg substitute composition. The composition may comprise: (a) recombinant egg-white proteins may consist of a recombinant ovomucoid (rOVD) and a recombinant ovalbumin (rOVA); (b) one or more gelation/thickening agents; (c) a salt and/or another flavoring agent; and (d) a lipid component; wherein a weight ratio of recombinant egg-white proteins to lipid component may be greater than 1:1.

In some embodiments, a weight ratio of rOVD and rOVA may be from about 1:50 to about 2:1. In some embodiments, the weight percent of protein to composition may be greater than about 10% on a w/w basis. In some embodiments, the weight percent of protein to composition may be less than about 95% on a w/w basis. In some embodiments, the composition lacks any animal-derived substances or any animal-derived components.

In some embodiments, a weight ratio of rOVD and rOVA may be less than about 1:50, may be less than about 1:40, may be less than about 1:30, may be less than about 1:20, may be less than about 1:10, may be less than about 1:5, may be less than about 1:4, may be less than about 1:3, may be less than about 1:2, less than about 1:1, or may be less than about 2:1. In some embodiments, the weight percent of rOVA to composition may be from about 9% to about 86% on a w/w basis. In some embodiments, the rOVA has one or more N-linked glycosylation sites having mannose linked to an N-acetyl glucosamine, and wherein the N-linked glycosylation sites lack galactose. In some embodiments, the rOVA has at least 80%, 85%, 90%, 92%, 95%, 96%, 97%, 98%, 99% of 100% sequence identity to any one of SEQ ID NO: 45 to SEQ ID NO: 118. In some embodiments, the weight percent of rOVD to composition may be from about 0.6% to about 50% on a w/w basis.

In some embodiments, the rOVD comprises a glycosylation pattern that differs from the glycosylation pattern of a native chicken ovomucoid. In some embodiments, the rOVD comprises at least one glycosylated asparagine residue. In some embodiments, the rOVD may be substantially devoid of N-linked mannosylation. In some embodiments, the rOVD has at least 80%, 85%, 90%, 92%, 95%, 96%, 97%, 98%, 99% of 100% sequence identity to any one of SEQ ID NO. 1 to SEQ ID NO: 44.

In some embodiments, when the composition may be combined with a liquid to form a liquid whole egg substitute composition, and when the liquid whole egg substitute composition and a whole hen's egg are prepared as a scramble, the scrambled whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the composition may be combined with a liquid to form a liquid whole egg substitute composition, and when the liquid whole egg substitute composition and a liquid composition may comprise a protein component may consist of proteins obtained from a plant are prepared as a scramble, the scrambled whole egg substitute composition provides better sensory attributes than those of a scrambled composition may comprise a protein component may consist of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, the amino acid profile of the recombinant egg-white proteins may be closer to a whole hen's egg than the amino acid profile of a protein component may consist of proteins obtained from a plant. In some embodiments, the nutrition value provided by amino acids of the recombinant egg-white proteins may be closer to a whole hen's egg than the nutrition value provided by amino acids of a protein component may consist of proteins obtained from a plant. In some embodiments, the recombinant egg-white protein comprises a fraction of cysteine, methionine, and/or lysine amino acids that may be closer to the fraction in a whole hen's egg than the fraction in a protein component may consist of proteins obtained from a plant.

In some embodiments, the recombinant egg-white protein comprises a larger fraction of cysteine, methionine, and/or lysine amino acids than the fraction in a composition may comprise a protein component may consist of proteins obtained from a plant. In some embodiments, the recombinant egg-white protein comprises a fraction of cysteine and methionine amino acids closer to the fraction in a whole hen's egg than the fraction in a protein component may consist of proteins obtained from a plant. In some embodiments, the fraction of cysteine, methionine, and/or lysine amino acids in the recombinant egg-white proteins provides, in part, a flavor and/or smell that approximates the flavor and/or smell of a whole hen's egg.

In some embodiments, the fraction of cysteine, methionine, and/or lysine amino acids in the recombinant egg-white proteins provides, in part, a flavor and/or smell that may be superior to the flavor and/or smell of composition may comprise a protein component may consist of proteins obtained from a plant. In some embodiments, the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein. In some embodiments, the proteins obtained from a plant comprise or consist of chickpea protein and mung bean protein or the proteins obtained from a plant comprise or consist of lupin bean protein and pea protein.

In some aspects, provided herein is a liquid whole egg substitute composition. In some embodiments, the composition may comprise: (a) recombinant egg-white proteins may comprise a recombinant ovomucoid (rOVD) and/or a recombinant ovalbumin (rOVA); (b) one or more gelation/thickening agents; (c) a salt and/or another flavoring agent; (d) a lipid component; and (e) water; wherein a weight ratio of recombinant egg-white proteins to lipid component may be greater than 1:1.

In some embodiments, a weight ratio of rOVD and rOVA may be from about 1:50 to about 2:1. In some embodiments, the weight percent of protein to composition may be greater than about 2% on a w/w basis. In some embodiments, the weight percent of protein to composition may be less than about 20% on a w/w basis. In some embodiments, the composition lacks any animal-derived substances or any animal-derived components. In some embodiments, a weight ratio of rOVD and rOVA may be less than about 1:50, may be less than about 1:40, may be less than about 1:30, may be less than about 1:20, may be less than about 1:10, may be less than about 1:5, may be less than about 1:4, may be less than about 1:3, may be less than about 1:2, less than about 1:1, or may be less than about 2:1.

In some embodiments, the weight percent of rOVA to composition may be from about 2% to about 10% on a w/w basis. In some embodiments, the rOVA has one or more N-linked glycosylation sites having mannose linked to an N-acetyl glucosamine, and wherein the N-linked glycosylation sites lack galactose. In some embodiments, the rOVA has at least 80%, 85%, 90%, 92%, 95%, 96%, 97%, 98%, 99% of 100% sequence identity to any one of SEQ ID NO: 45 to SEQ ID NO: 118. In some embodiments, the weight percent of rOVD to composition may be from about 0.15% to about 4.5% on a w/w basis. In some embodiments, the rOVD comprises a glycosylation pattern that differs from the glycosylation pattern of a native chicken ovomucoid.

In some embodiments, the rOVD comprises at least one glycosylated asparagine residue. In some embodiments, the rOVD may be substantially devoid of N-linked mannosylation. In some embodiments, the rOVD has at least 80%, 85%, 90%, 92%, 95%, 96%, 97%, 98%, 99% of 100% sequence identity to any one of SEQ ID NO. 1 to SEQ ID NO: 44. In some embodiments, when the composition and a whole hen's egg are prepared as a scramble, the scrambled composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the composition and a composition may comprise a protein component may consist of proteins obtained from a plant are prepared as a scramble, the scrambled composition provides better sensory attributes than those of a scrambled composition may comprise a protein component may consist of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the amino acid profile of the recombinant egg-white proteins may be closer to a whole hen's egg than the amino acid profile of a protein component may consist of proteins obtained from a plant. In some embodiments, the nutrition value provided by amino acids of the recombinant egg-white proteins may be closer to a whole hen's egg than the nutrition value provided by amino acids of a protein component may consist of proteins obtained from a plant. In some embodiments, the recombinant egg-white protein comprises a fraction of cysteine, methionine, and/or lysine amino acids that may be closer to the fraction in a whole hen's egg than the fraction in a protein component may consist of proteins obtained from a plant.

In some embodiments, the recombinant egg-white protein comprises a larger fraction of cysteine, methionine, and/or lysine amino acids than the fraction in a composition may comprise a protein component may consist of proteins obtained from a plant. In some embodiments, the fraction of cysteine, methionine, and/or lysine amino acids in the recombinant egg-white proteins provides, in part, a flavor and/or smell that approximates the flavor and/or smell of a whole hen's egg.

In some embodiments, the fraction of cysteine, methionine, and/or lysine amino acids in the recombinant egg-white proteins provides, in part, a flavor and/or smell that may be superior to the flavor and/or smell of composition may comprise a protein component may consist of proteins obtained from a plant. In some embodiments, the composition further comprises one or more proteins obtained from a plant. In some embodiments, the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein. In some embodiments, the proteins obtained from a plant comprise or consist of chickpea protein and mung bean protein or the proteins obtained from a plant comprise or consist of lupin bean protein and pea protein. In some embodiments, the recombinant egg-white proteins further comprises recombinant lysozyme (rOVL).

In some embodiments, the weight percent of rOVL to composition may be from about 0.1% to about 5% on a w/w basis. In some embodiments, the rOVL may be a recombinant chicken egg white lysozyme (cOVL) or a recombinant goose lysozyme (gOVL). In some embodiments, the rOVL has at least 80%, 85%, 90%, 92%, 95%, 96%, 97%, 98%, 99% of 100% sequence identity to any one of SEQ ID NO: 119 to SEQ ID NO: 125.

In some aspects, provided herein is a powdered whole egg substitute composition. In some embodiments, the composition may comprise: (a) recombinant egg-white proteins may comprise a recombinant ovomucoid (rOVD) and/or a recombinant ovalbumin (rOVA); (b) one or more gelation/thickening agents; (c) a salt and/or another flavoring agent; and (d) a lipid component wherein a weight ratio of recombinant egg-white proteins to lipid component may be greater than 1:1. In some embodiments, a weight ratio of rOVD and rOVA may be from about 1:50 to about 2:1.

In some embodiments, the weight percent of protein to composition may be greater than about 10% on a w/w basis. In some embodiments, the weight percent of protein to composition may be less than about 95% on a w/w basis. In some embodiments, the composition lacks any animal-derived substances or any animal-derived components.

In some embodiments, a weight ratio of rOVD and rOVA may be less than about 1:50, may be less than about 1:40, may be less than about 1:30, may be less than about 1:20, may be less than about 1:10, may be less than about 1:5, may be less than about 1:4, may be less than about 1:3, may be less than about 1:2, less than about 1:1, or may be less than about 2:1. In some embodiments, the weight percent of rOVA to composition may be from about 9% to about 86% on a w/w basis. In some embodiments, the rOVA has one or more N-linked glycosylation sites having mannose linked to an N-acetyl glucosamine, and wherein the N-linked glycosylation sites lack galactose. In some embodiments, the rOVA has at least 80%, 85%, 90%, 92%, 95%, 96%, 97%, 98%, 99% of 100% sequence identity to any one of SEQ ID NO: 45 to SEQ ID NO: 118.

In some embodiments, the weight percent of rOVD to composition may be from about 0.6% to about 50% on a w/w basis. In some embodiments, the rOVD comprises a glycosylation pattern that differs from the glycosylation pattern of a native chicken ovomucoid. In some embodiments, the rOVD comprises at least one glycosylated asparagine residue. In some embodiments, the rOVD may be substantially devoid of N-linked mannosylation. In some embodiments, the rOVD has at least 80%, 85%, 90%, 92%, 95%, 96%, 97%, 98%, 99% of 100% sequence identity to any one of SEQ ID NO. 1 to SEQ ID NO: 44.

In some embodiments, when the composition may be combined with a liquid to form a liquid whole egg substitute composition, and when the liquid whole egg substitute composition and a whole hen's egg are prepared as a scramble, the scrambled whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the composition may be combined with a liquid to form a liquid whole egg substitute composition, and when the liquid whole egg substitute composition and a liquid composition may comprise a protein component may consist of proteins obtained from a plant are prepared as a scramble, the scrambled whole egg substitute composition provides better sensory attributes than those of a scrambled composition may comprise a protein component may consist of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, the amino acid profile of the recombinant egg-white proteins may be closer to a whole hen's egg than the amino acid profile of a protein component may consist of proteins obtained from a plant. In some embodiments, the nutrition value provided by amino acids of the recombinant egg-white proteins may be closer to a whole hen's egg than the nutrition value provided by amino acids of a protein component may consist of proteins obtained from a plant. In some embodiments, the recombinant egg-white protein comprises a fraction of cysteine, methionine, and/or lysine amino acids that may be closer to the fraction in a whole hen's egg than the fraction in a protein component may consist of proteins obtained from a plant. In some embodiments, the recombinant egg-white protein comprises a larger fraction of cysteine, methionine, and/or lysine amino acids than the fraction in a composition may comprise a protein component may consist of proteins obtained from a plant.

In some embodiments, the fraction of cysteine, methionine, and/or lysine amino acids in the recombinant egg-white proteins provides, in part, a flavor and/or smell that approximates the flavor and/or smell of a whole hen's egg. In some embodiments, the fraction of cysteine, methionine, and/or lysine amino acids in the recombinant egg-white proteins provides, in part, a flavor and/or smell that may be superior to the flavor and/or smell of composition may comprise a protein component may consist of proteins obtained from a plant. In some embodiments, the composition further comprises one or more proteins obtained from a plant.

In some embodiments, the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein. In some embodiments, the proteins obtained from a plant comprise or consist of chickpea protein and mung bean protein or the proteins obtained from a plant comprise or consist of lupin bean protein and pea protein. In some embodiments, the recombinant egg-white proteins further comprises recombinant lysozyme (rOVL).

In some embodiments, the weight percent of rOVL to composition may be from about 0.1% to about 15% on a w/w or w/v basis. In some embodiments, the rOVL may be a recombinant chicken egg white lysozyme (cOVL) or a recombinant goose lysozyme (gOVL). In some embodiments, the rOVL has at least 80%, 85%, 90%, 92%, 95%, 96%, 97%, 98%, 99% of 100% sequence identity to any one of SEQ ID NO: 119 to SEQ ID NO: 125. In some embodiments, the recombinant egg-white proteins are expressed in Pichia pastoris. In some embodiments, the one or more gelation agents comprises one or more polysaccharide-based hydrocolloids or protein-based hydrocolloids.

In some embodiments, the one or more polysaccharide or protein-based hydrocolloids comprises a beta-glucan (such as a bacterial beta-glucan, barley beta-glucan, Betafectin/TH-glucan, botryosphaeran, callose, carboxymethylpachymaran, cereal beta-glucan, cerevan, chitin-glucan, chrysolaminarin, coriolan, curdlan, epiglucan, fungal beta-glucan, grifolan, krestin, laminaran/laminarin, latiglucan, lentinan, leucosin, lichenan/lichenin, mycolaminarin, oat beta-glucan, pachymaran/pachyman, paramylon, pendulan, pestalotan, phycarine, pleuran, polycan, polysaccharide-glucan, pustulan, scleroglucan/sclero-beta-glucan, sclerotinan/sclerotan, tylopilan, yeast beta-glucan, yestimun, and zymosan), gellan gum (e.g., high acyl gellan gum and low acyl gellan gum), guar gum, locust bean gum, xanthan gum, carageenan (e.g., kappa carrageenan and iota carrageenan), alginate, sodium alginate, agar, gum arabic, lecithin, gelatin, pectin, Psyllium, corn starch, potato starch, rice starch, tapioca starch, modified starch, carboxy methylcellulose, methylcellulose, hydroxypropyl methylcullose, konjac gum, or transglutaminase.

In some embodiments, the polysaccharide-based hydrocolloids comprises a beta-glucan. In some embodiments, the beta-glucan is a bacterial beta-glucan, fungal beta-glucan, yeast beta-glucan or cereal beta-glucan such as an oat beta-glucan, or a specific beta-glucan listed in the previous paragraph. In some embodiments, the polysaccharide-based hydrocolloids comprises high acyl gellan gum or low acyl gellan gum. In some embodiments, the polysaccharide-based hydrocolloids comprises a beta-glucan and a gellan gum. In some embodiments, the weight percent of the one or more gelation agents to composition may be from about 0.5% to about 5% on a w/w or w/v basis. In some embodiments, the salt comprises white salt, black salt, or Himalayan black salt (e.g., Rock salts (such as kala namak)) and/or comprises a Na+, Ca+2, K+, or Mg+2 cation, optionally, wherein the salt serves as a cross-linking agent.

In some embodiments, the salt comprises Rock salts (such as kala namak). In some embodiments, the weight percent of the salt to composition may be from about 0.1% to about 2% on a w/w or w/v basis. In some embodiments, the other flavoring agent comprises a natural or synthetic flavoring. In some embodiments, the synthetic flavoring comprises synthetic egg yolk flavor. In some embodiments, the weight percent of the other flavoring agent to composition may be from about 0.1% to about 5% on a w/w or w/v basis. In some embodiments, the lipid component comprises one or more saturated vegetable oils or unsaturated vegetable oils.

In some embodiments, the one or more saturated vegetable oils or unsaturated vegetable oils comprises coconut oil, palm oil, palm kernel oil, canola oil, soybean oil, corn oil, cottonseed oil, olive oil, flaxseed oil, sunflower oil, safflower oil, peanut oil, or avocado oil. In some embodiments, the one or more saturated vegetable oils or unsaturated vegetable oils are in their natural state or are chemically or enzymatically processed. In some embodiments, the chemically or enzymatically processing produces an interesterified oil. In some embodiments, the saturated vegetable oils or unsaturated vegetable oil comprises one or more of coconut oil, palm oil, and palm kernel oil. In some embodiments, the saturated vegetable oils or unsaturated vegetable oil comprises two or more of coconut oil, palm oil, and palm kernel oil. In some embodiments, the saturated vegetable oils or unsaturated vegetable oil comprises each of coconut oil, palm oil, and palm kernel oil.

In some embodiments, the weight percent of the lipid component to composition may be from about 2% to about 15% on a w/w or w/v basis. In some embodiments, the composition further comprises one or more thickening agents. In some embodiments, the one or more thickening agents comprises corn starch, potato starch, arrowroot starch, rice starch, tapioca starch, tapioca syrup, rice syrup, modified starch, carboxymethylcellulose, guar gum, locust bean gum, xanthan gum, carrageenan, gum Arabic, and Psyllium.

In some embodiments, the one or more thickening agents comprises one or more of tapioca syrup, Psyllium, and xanthan gum. In some embodiments, the one or more thickening agents comprises two or more of tapioca syrup, Psyllium, and xanthan gum. In some embodiments, the one or more thickening agents comprises each of tapioca syrup, Psyllium, and xanthan gum. In some embodiments, the weight percent of the one or more thickening agents to composition may be from about 0.1% to about 30% on a w/w basis. In some embodiments, the composition further comprises one or more natural or synthetic coloring. In some embodiments, the one or more natural or synthetic coloring may be pineapple yellow. In some embodiments, the weight percent of the one or more natural or synthetic coloring to composition may be from about 0.1% to about 2% on a w/w basis.

In some embodiments, the composition further comprises one or more a natural emulsifiers or synthetic emulsifiers. In some embodiments, the one or more a natural emulsifiers or synthetic emulsifiers comprises soy or sunflower lecithin, mono- and diglycerides, ethoxylated mono- and diglycerides, polyglycerol esters, sugar esters, polysorbate, and sorbitan. In some embodiments, the one or more a natural emulsifiers or synthetic emulsifiers comprises sunflower lecithin. In some embodiments, the weight percent of the one or more natural or synthetic coloring to composition may be from about 0.1% to about 2% on a w/w basis. In some embodiments, the composition further comprises one or more dietary fiber-containing component comprises one or more of Psyllium husk fiber, Bamboo fiber, oat fiber, carrot fiber, flaxseed, chia seed, wheat fiber, pea fiber, potato fiber, apple fiber, citrus fiber, accacia fiber, and cellulose fiber.

In some embodiments, the dietary fiber-containing component may be present in the substantially liquid mixture in a concentration from about 0.1% to about 10% on a weight per weight or weight per volume basis. In some embodiments, the dietary fiber-containing component comprises Psyllium husk fiber. In some embodiments, the weight percent of the Psyllium husk fiber to composition may be from about 0.1% to about 5% on a w/w or w/v basis. In some embodiments, the weight percent of the Psyllium husk fiber to composition may be about 0.7% on a w/w or w/v basis.

In some embodiments, the composition further comprises a flour. In some embodiments, the composition further comprises a leavening agent. In some embodiments, the leavening agent may be baking powder, yeast or baking soda. In some embodiments, when the composition may be a liquid, the composition further comprises a syrup component.

In some embodiments, the syrup component comprises honey, high fructose corn syrup, high maltose corn syrup, corn syrup (e.g., glucose-free corn syrup), simple syrup (e.g., may comprise sucrose), sweet potato syrup, tapioca syrup, maple syrup, agave syrup, cane syrup, golden syrup, or brown rice syrup, or a combination thereof. In some embodiments, the weight percent of the syrup component to composition may be from about 0.1% to about 5%, from about 0.3% to about 2%, or from about 0.5% to about 1.5% on a w/w or w/v basis. In some embodiments, when the composition may be a liquid, the weight percent of the water to composition may be from about 25% to about 90%, about 50% to about 85%, or from about 65% to about 80% on a w/w or w/v basis. In some embodiments, the composition has a shelf-life of greater than 3, 4, 5, 6, or 7 days at a refrigerated temperature of about 37° F.

In some aspects, provided herein is a liquid whole egg substitute composition. The composition may comprise: (a) recombinant egg-white proteins may consist of a recombinant ovomucoid (rOVD) and a recombinant ovalbumin (rOVA); (b) one or more gelation agents; (c) a salt and/or another flavoring agent; (d) a lipid component; (e) one or more thickening agents; (f) one or more natural or synthetic coloring; (g) one or more a natural emulsifiers or synthetic emulsifiers; and (h) water; wherein a weight ratio of recombinant egg-white proteins to lipid component may be greater than 1:1.

In some embodiments, a weight ratio of rOVD and rOVA may be from about 1:50 to about 2:1. In some embodiments, the weight percent of protein to composition may be greater than about 2% on a w/w basis. In some embodiments, the weight percent of protein to composition may be less than about 15% on a w/w basis. In some embodiments, the composition lacks any animal-derived substances or any animal-derived components. In some embodiments, a weight ratio of rOVD and rOVA may be less than about 1:50, may be less than about 1:40, may be less than about 1:30, may be less than about 1:20, may be less than about 1:10, may be less than about 1:5, may be less than about 1:4, may be less than about 1:3, may be less than about 1:2, less than about 1:1, or may be less than about 2:1.

In some embodiments, the weight percent of rOVA to composition may be from about 2% to about 10% on a w/w basis. In some embodiments, the rOVA has one or more N-linked glycosylation sites having mannose linked to an N-acetyl glucosamine, and wherein the N-linked glycosylation sites lack galactose. In some embodiments, the rOVA has at least 80%, 85%, 90%, 92%, 95%, 96%, 97%, 98%, 99% of 100% sequence identity to any one of SEQ ID NO: 45 to SEQ ID NO: 118. In some embodiments, the weight percent of rOVD to composition may be from about 0.15% to about 4.5% on a w/w basis. In some embodiments, the rOVD comprises a glycosylation pattern that differs from the glycosylation pattern of a native chicken ovomucoid.

In some embodiments, the rOVD comprises at least one glycosylated asparagine residue. In some embodiments, the rOVD may be substantially devoid of N-linked mannosylation. In some embodiments, the rOVD has at least 80%, 85%, 90%, 92%, 95%, 96%, 97%, 98%, 99% of 100% sequence identity to any one of SEQ ID NO. 1 to SEQ ID NO: 44. In some embodiments, when the composition and a whole hen's egg are prepared as a scramble, the scrambled composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the composition and a composition may comprise a protein component may consist of proteins obtained from a plant are prepared as a scramble, the scrambled composition provides better sensory attributes than those of a scrambled composition may comprise a protein component may consist of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, the amino acid profile of the recombinant egg-white proteins may be closer to a whole hen's egg than the amino acid profile of a protein component may consist of proteins obtained from a plant.

In some embodiments, the nutrition value provided by amino acids of the recombinant egg-white proteins may be closer to a whole hen's egg than the nutrition value provided by amino acids of a protein component may consist of proteins obtained from a plant. In some embodiments, the recombinant egg-white protein comprises a fraction of cysteine, methionine, and/or lysine amino acids that may be closer to the fraction in a whole hen's egg than the fraction in a protein component may consist of proteins obtained from a plant. In some embodiments, the recombinant egg-white protein comprises a larger fraction of cysteine, methionine, and/or lysine amino acids than the fraction in a composition may comprise a protein component may consist of proteins obtained from a plant.

In some embodiments, the fraction of cysteine, methionine, and/or lysine amino acids in the recombinant egg-white proteins provides, in part, a flavor and/or smell that approximates the flavor and/or smell of a whole hen's egg. In some embodiments, the fraction of cysteine, methionine, and/or lysine amino acids in the recombinant egg-white proteins provides, in part, a flavor and/or smell that may be superior to the flavor and/or smell of composition may comprise a protein component may consist of proteins obtained from a plant. In some embodiments, the composition further comprises one or more proteins obtained from a plant. In some embodiments, the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein. In some embodiments, the proteins obtained from a plant comprise or consist of chickpea protein and mung bean protein or the proteins obtained from a plant comprise or consist of lupin bean protein and pea protein.

In some embodiments, the one or more gelation agents comprises a beta-glucan and/or a gellan gum. In some embodiments, the salt comprises Rock salts (such as kala namak). In some embodiments, the other flavoring agent comprises synthetic egg yolk flavor. In some embodiments, the lipid component comprises one or more, two more, or each of coconut oil, palm oil, and palm kernel oil. In some embodiments, the one or more thickening agents comprises one or more, two or more of, or each of tapioca syrup, Psyllium, and xanthan gum.

In some embodiments, the one or more natural or synthetic coloring may be pineapple yellow.

In some embodiments, the one or more a natural emulsifiers or synthetic emulsifiers comprises sunflower lecithin.

In some aspects, provided herein is a powdered whole egg substitute composition. The composition may comprise: (a) recombinant egg-white proteins may consist of a recombinant ovomucoid (rOVD) and a recombinant ovalbumin (rOVA); (b) one or more gelation agents; (c) a salt and/or another flavoring agent; and (d) a lipid component; (e) one or more thickening agents; (f) one or more natural or synthetic coloring; and (g) one or more a natural emulsifiers or synthetic emulsifiers; wherein a weight ratio of recombinant egg-white proteins to lipid component may be greater than 1:1.

In some embodiments, a weight ratio of rOVD and rOVA may be from about 1:50 to about 2:1. In some embodiments, the weight percent of protein to composition may be greater than about 10% on a w/w basis. In some embodiments, the weight percent of protein to composition may be less than about 95% on a w/w basis. In some embodiments, the composition lacks any animal-derived substances or any animal-derived components.

In some embodiments, a weight ratio of rOVD and rOVA may be less than about 1:50, may be less than about 1:40, may be less than about 1:30, may be less than about 1:20, may be less than about 1:10, may be less than about 1:5, may be less than about 1:4, may be less than about 1:3, may be less than about 1:2, less than about 1:1, or may be less than about 2:1.

In some embodiments, the weight percent of rOVA to composition may be from about 9% to about 86% on a w/w basis. In some embodiments, the rOVA has one or more N-linked glycosylation sites having mannose linked to an N-acetyl glucosamine, and wherein the N-linked glycosylation sites lack galactose. In some embodiments, the rOVA has at least 80%, 85%, 90%, 92%, 95%, 96%, 97%, 98%, 99% of 100% sequence identity to any one of SEQ ID NO: 45 to SEQ ID NO: 118. In some embodiments, the weight percent of rOVD to composition may be from about 0.6% to about 50% on a w/w basis.

In some embodiments, the rOVD comprises a glycosylation pattern that differs from the glycosylation pattern of a native chicken ovomucoid. In some embodiments, the rOVD comprises at least one glycosylated asparagine residue. In some embodiments, the rOVD may be substantially devoid of N-linked mannosylation. In some embodiments, the rOVD has at least 80%, 85%, 90%, 92%, 95%, 96%, 97%, 98%, 99% of 100% sequence identity to any one of SEQ ID NO. 1 to SEQ ID NO: 44.

In some embodiments, when the composition may be combined with a liquid to form a liquid whole egg substitute composition, and when the liquid whole egg substitute composition and a whole hen's egg are prepared as a scramble, the scrambled whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, when the composition may be combined with a liquid to form a liquid whole egg substitute composition, and when the liquid whole egg substitute composition and a liquid composition may comprise a protein component may consist of proteins obtained from a plant are prepared as a scramble, the scrambled whole egg substitute composition provides better sensory attributes than those of a scrambled composition may comprise a protein component may consist of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

In some embodiments, the amino acid profile of the recombinant egg-white proteins may be closer to a whole hen's egg than the amino acid profile of a protein component may consist of proteins obtained from a plant. In some embodiments, the nutrition value provided by amino acids of the recombinant egg-white proteins may be closer to a whole hen's egg than the nutrition value provided by amino acids of a protein component may consist of proteins obtained from a plant. In some embodiments, the recombinant egg-white protein comprises a fraction of cysteine, methionine, and/or lysine amino acids that may be closer to the fraction in a whole hen's egg than the fraction in a protein component may consist of proteins obtained from a plant. In some embodiments, the recombinant egg-white protein comprises a larger fraction of cysteine, methionine, and/or lysine amino acids than the fraction in a composition may comprise a protein component may consist of proteins obtained from a plant.

In some embodiments, the fraction of cysteine, methionine, and/or lysine amino acids in the recombinant egg-white proteins provides, in part, a flavor and/or smell that approximates the flavor and/or smell of a whole hen's egg. In some embodiments, the fraction of cysteine, methionine, and/or lysine amino acids in the recombinant egg-white proteins provides, in part, a flavor and/or smell that may be superior to the flavor and/or smell of composition may comprise a protein component may consist of proteins obtained from a plant. In some embodiments, the composition further comprises one or more proteins obtained from a plant. In some embodiments, the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

In some embodiments, the proteins obtained from a plant comprise or consist of chickpea protein and mung bean protein or the proteins obtained from a plant comprise or consist of lupin bean protein and pea protein. In some embodiments, the one or more gelation agents comprises a beta-glucan and/or a gellan gum. In some embodiments, the salt comprises Rock salts (such as kala namak).

In some embodiments, the other flavoring agent comprises synthetic egg yolk flavor. In some embodiments, the lipid component comprises one or more, two more, or each of coconut oil, palm oil, and palm kernel oil. In some embodiments, the one or more thickening agents comprises one or more, two or more of, or each of tapioca syrup, Psyllium, and xanthan gum.

In some embodiments, the one or more natural or synthetic coloring may be pineapple yellow. In some embodiments, the one or more a natural emulsifiers or synthetic emulsifiers comprises sunflower lecithin. In some embodiments, the food product may be a baked product selected from the group may consist of cake (e.g., pound cake, sponge cake, yellow cake, or angel food cake), cookie, bagel, biscuit, bread, muffin, crepe, cupcake, scone, pancake, macaron, macaroon, meringue, choux pastry, and soufflé; a batter; a beverage selected from the group may consist of smoothie, milkshake, “egg-nog”, and coffee beverage; a confectionary selected from a gummy, a taffy, a chocolate, or a nougat; a dessert product selected from the group may consist of a mousse, a cheesecake, a custard, a pudding, a popsicle, a frozen dessert, and an ice cream; a food emulsion; a meat analog food product selected from a burger, patty, sausage, hot dog, sliced deli meat, jerky, bacon, nugget, a ground meat-like composition, and a formed meat-like composition; a noodle; a pasta; a pet food; a sauce or dressing selected from the group may consist of salad dressing, mayonnaise, commercial mayonnaise substitutes, alfredo sauce, and hollandaise sauce; a snack food selected from a protein bar, a nutrition bar, or a granola bar; a yoghurt; an egg-wash; or egg-like dish selected from the group may consist of scramble, omelet, patty, soufflé, quiche, and frittata.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A are schematics comparing the glycosylation pattern of native ovomucoid and a recombinant ovomucoid produced in P. pastoris. Shown is a lack of the complex branched glycosylation (including a lack of mannose residues) on the recombinant ovomucoid when produced in a strain of P. pastoris comprising endoglycosidases.

FIG. 1B is a mass spectrometry analysis showing the glycosylation patterns of the recombinant ovomucoid (rOVD) produced by P. pastoris without an endoglycosidase treatment. rOVD thus produced have complex branched glycosylation patterns.

FIG. 1C is a gel comparing the molecular weight of native ovomucoid (nOVD), nOVD treated with an endoglycosidase (here, PNGaseF), and rOVD samples.

FIG. 2A to FIG. 2B, respectively, are schematics illustrating glycosylation patterns of native OVA and rOVA produced in P. pastoris respectively.

FIG. 2C is a gel showing the electrophoresis migration of glycosylated native and recombinant ovalbumin (rOVA). Also shown are deglycosylated rOVA treated with EndoH (EH) or PNGaseF (PF).

FIG. 2D is a chromatogram depicting glycosylation patterns of rOVA produced in P. pastoris.

FIG. 3A are photographs of gels formed from (left) 9% rOVA; (center) 6% rOVA and 6% rOVD; and (right) whole egg.

FIG. 3B is a photograph of a gel formed from 6% rOVA and 3% rOVD.

FIG. 3C is a photograph of a gel formed from fresh egg white.

FIG. 4A is a photograph of three scramble compositions formed from 6% rOVA and 3% rOVD (Sample A), 6% rOVA and 6% rOVD (Sample B), and a control made from fresh egg (Control).

FIG. 4B is a photograph of a scramble composition formed from 6% rOVA.

FIG. 5 is a photograph of four different scramble compositions formed from chickpea protein (Sample 1008), chickpea protein and mungbean protein (Sample 1005), chickpea protein and nOVD (Sample 1006), and chickpea protein and rOVD (Sample 1007).

FIGS. 6 and 7 are photographs of different scramble compositions produced.

### DETAILED DESCRIPTION OF THE DISCLOSURE

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are compositions, methods for using compositions, and methods of making compositions comprising a substantially liquid mixture including recombinant proteins, which may be used for preparing, and/or replacing, an egg or egg-like product to be used alone and/or in combination with other food ingredients for consumption. Various egg substitute compositions are commercially available to consumers, but they do not match the flavor profile, amino acid profile, nutritional profile, texture/taste profile and functionality of an egg-protein based substitute composition. It is an unexpected effect of the present disclosure that using one or more egg-white proteins (recombinantly mode, and preferably present in combination) in an egg-replacing composition such as described herein, a consumer may be able to effectively replace a native egg/egg-white.

### Composition Comprising Substantially Liquid Mixtures

Provided herein, in certain embodiments, are compositions comprising a substantially liquid/powdered mixture including recombinant proteins. The substantially liquid/powdered mixture may comprise ingredients of one or more egg-related proteins selected from the group consisting of a recombinant ovomucoid (rOVD), and a recombinant ovalbumin (rOVA), and a recombinant lysozyme; a protein component, wherein the protein component comprises a plant protein; a dietary fiber-providing component, wherein the dietary fiber-providing component comprises plant fiber; a starch-providing component, the starch-providing component comprising polysaccharides having glucose monomers, e.g., joined via α-1,4 linkages; a gelation agent; a salt and/or another flavoring agent; a lipid component; and water.

In various embodiments, the substantially liquid/powdered mixture comprises (a) one or more egg-related proteins selected from the group consisting of a recombinant ovomucoid (rOVD), and a recombinant ovalbumin (rOVA), and a recombinant lysozyme; (b) a protein component, wherein the protein component comprises a plant protein; (c) a dietary fiber-providing component, wherein the dietary fiber-providing component comprises plant fiber; (d) a starch-providing component, the starch-providing component comprising polysaccharides having glucose monomers, e.g., joined via α-1,4 linkages; (e) a gelation agent; (f) a salt and/or another flavoring agent; (g) a lipid component; and (h) water. In some embodiments, the substantially liquid mixture comprises components (a), (b), (c), (d), (e), (f), (g), and (h). In some embodiments, the substantially liquid composition comprises at least components (a) and/or (b). In some embodiments, the substantially liquid mixture comprises components (a)-(h). In some embodiments, the substantially liquid mixture comprises (a) and any one or more of (b)-(h). In some embodiments, the substantially liquid mixture comprises (a), (b), and any one or more of (c)-(h). In some embodiments, the composition comprises (a) and two or more of (b), (c), (d), (e), (f), (g), and (h), e.g., three or more, four or more, five or more, six or more, or seven, of (b), (c), (d), (e), (f), (g), and (h). In some embodiments, the substantially liquid mixture comprises (b) and any one or more of (c)-(h). In some embodiments, the composition comprises (b) and two or more of (c), (d), (e), (f), (g), and (h), e.g., three or more, four or more, five or more, or six of (b), (c), (d), (e), (f), (g), and (h). In some embodiments, the substantially liquid mixture comprises (a), (b), (c), (d), (e), (f), (g), and/or (h), and any combination thereof.

The compositions comprising a substantially liquid/powdered mixture may be used for preparing, and/or replacing, an egg-like product. As used herein, the term “replacing” and variations thereof, refer to wholly substituting for a different component, or substituting for only a part of a different component. For example, if the substantially liquid/powdered mixture is replacing an egg or egg white, the substantially liquid/powdered mixture may wholly replace the egg or egg white, or the substantially liquid/powdered mixture may partially replace the egg or egg white so that a portion of egg or egg white is still present. For instance, a liquid composition may be used as a whole egg whereas a powdered composition may be diluted with water to replace an egg. As used herein, the term “egg-like product” and variations thereof, refers to a product sharing certain characteristics with eggs (i.e. taste, hardness, cohesiveness, chewiness, gelling capability, etc.). As used herein, “egg,” “egg whites,” and variants thereof, refer to a chicken egg (i.e., hen egg), ostrich egg, quail egg, duck egg, or any other type of naturally occurring edible egg, and does not include proteins that are produced recombinantly.

The compositions comprising a substantially liquid/powdered mixture may be used in or as a food product or beverage. In some embodiments, the composition is used in conjunction with eggs to make an egg containing product. In some embodiments, the composition is used in making an egg-less food product. In some embodiments, the egg-less food product is a scramble, such as an egg-less vegan scramble. As used herein, the term “vegan” refers to the absence of animal products.

In some embodiments, a composition is a substantially liquid mixture. The term “substantially liquid” includes, but is not limited to, a generally fluid mixture, that may or may not include solid particulates and/or gasses. The “substantially liquid mixture” may be capable of flow and may or may not have solid domains therein. As used herein, the term “substantially liquid mixture” is used synonymously with “liquid mixture.” The composition may be used to produce a liquid, solid, or semi-solid consumable product, e.g., by heating or by freezing.

In some embodiments, a substantially liquid/powdered mixture comprises one or more egg-related protein. As used herein, the term “egg-related protein” refers to proteins that are found in an egg. Examples of egg-related proteins include ovomucoid (OVD), ovalbumin (OVA), lysozyme (OVL), and ovotransferrin (OVT). In some embodiments, the egg-related protein is a native egg protein which has been isolated from a natural egg. The egg-related protein may be obtained from the egg of a chicken, ostrich, quail, duck, goose, turkey, pheasant, turkey vulture, hummingbird, or another animal.

In other embodiments, an egg-related protein is a recombinant protein that is expressed by a host cell. In some embodiments, the host cell comprises a Pichia species, a Saccharomyces species, a Trichoderma species, a Pseudomonas species or an E. coli species. In some embodiments, the Pichia species comprises Pichia pastoris. In some embodiments, glycosylation patterns of the recombinant proteins expressed by a Pichia species differs from their native corresponding proteins. For example, the recombinant proteins produced in Pichia pastoris may be highly glycosylated.

In some embodiments, the recombinant egg-related protein is recombinant ovomucoid (rOVD), recombinant ovalbumin (rOVA), and recombinant lysozyme (rOVL)). The recombinant egg-related proteins are expressed by a host cell. In some embodiments, the host cell is from a Pichia species (e.g., Pichia pastoris). The recombinant egg-related protein may have an amino acid sequence identical to or a variant a natural egg protein from an egg of a chicken, ostrich, quail, duck, goose, turkey, pheasant, turkey vulture, hummingbird, or another animal. Also useful in the present disclosure proteins having sequence similarity or homology to an egg protein yet originating in other animal species; these predicted egg-proteins, isoforms of egg proteins, or “like” egg proteins are also useful. In example, a recombinant egg protein may be an OVD-like protein or an isoform of an OVD or predicted to be an OVD, yet from an animal species not yet characterized as expressing OVD.

In some embodiments, the host cell secretes the recombinant protein, with the recombinant protein being collected from a culturing medium in which the host cell is cultured. Recombinant proteins may be identical in sequence to the native protein, or the recombinant protein may be a variant of the native protein by having a sequence that differs from the native protein. The recombinant proteins expressed by the Pichia species may be structurally different from natural protein counterparts (i.e., natural egg proteins). A recombinant protein may have post-translational modifications that differ from a native protein. One example of a post-translational modification is glycosylation; a recombinant protein may be glycosylated whereas the native protein is not; or the recombinant protein has one glycosylation pattern or specific polysaccharide chains whereas the native protein has a different pattern or different chains. Specifically, the recombinant egg-related proteins (e.g., rOVA, rOVD, and rOVL) have a glycosylation pattern that differs from the glycosylation pattern of naturally occurring egg proteins. For example, rOVD comprises at least one glycosylated asparagine residue and is substantially devoid of N-linked mannosylation. Additionally, native OVA has one or more N-linked glycan structures such as N-acetylglucosamine units, galactose and N-linked mannose units whereas rOVA may lack galactose units in the N-linked glycosylation.

In some embodiments, the composition is devoid of any animal-derived proteins, e.g., a recombinant egg-related protein. In some embodiments, the composition is devoid of any egg-derived protein. In some embodiments, the composition is devoid of any protein derived from an animal or an egg lain therefrom. Such a composition may be considered a vegan composition, as long as no animal-derived substances are added to the composition.

In various embodiments, the recombinant egg-related protein, is present in the substantially liquid/powdered mixture at a concentration from about 0.1% to about 40% on a weight per weight (w/w) or weight per volume (w/v) basis. In embodiments, the recombinant egg-related protein is present in the substantially liquid/powdered mixture at a concentration of about 0.10%, 0.20%, 0.30%, 0.40%, 0.50%, 0.60%, 0.70%, 0.80%, 0.90%, or about 1.00% w/w or w/v. In some embodiments, the recombinant egg-related protein is present in the substantially liquid/powdered mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the recombinant egg-related protein is present in the substantially liquid/powdered mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v.

In some embodiments, one or more recombinant egg-related proteins are present in the substantially liquid mixture at a concentration of 2% to 15% w/w or w/v. In some embodiments, one or more recombinant egg-related proteins are present in the substantially liquid mixture at a concentration of at least 2% w/w or w/v. In some embodiments, one or more recombinant egg-related proteins are present in the substantially liquid mixture at a concentration of at most 15% w/w or w/v. In some embodiments, one or more recombinant egg-related proteins are present in the substantially liquid mixture at a concentration of 2% to 5%, 2% to 7%, 2% to 10%, 2% to 12%, 2% to 15%, 5% to 7%, 5% to 10%, 5% to 12%, 5% to 15%, 7% to 10%, 7% to 12%, 7% to 15%, 10% to 12%, 10% to 15%, or 12% to 15% w/w or w/v. In some embodiments, one or more recombinant egg-related proteins are present in the substantially liquid mixture at a concentration of about 2%, 5%, 7%, 10%, 12%, or 15% w/w or w/v. In some embodiments, one or more recombinant egg-related proteins are present in the substantially liquid mixture at a concentration of less than 5%, 7%, 10%, 12%, or 15% w/w or w/v. In some embodiments, one or more recombinant egg-related proteins are present in the substantially liquid mixture at a concentration of more than 2%, 5%, 7%, 10%, or 12% w/w or w/v.

In embodiments, a substantially liquid/powdered mixture comprises one or more recombinant egg-related protein, e.g., rOVA, rOVD, and rOVL. The one or more recombinant egg-related proteins can increase the protein content of a consumable food product or food ingredient derived from the substantially liquid/powdered mixture of the present disclosure. In some embodiments, the one or more recombinant egg-related proteins provide one or more functional characteristics to a consumable food product or food ingredient derived from the substantially liquid/powdered mixture of the present disclosure. Examples of such functional characteristics include gelling, foaming, whipping, fluffing, binding, springiness, aeration, coating, film forming, emulsification, browning, thickening, texturizing, humectant, clarification, and cohesiveness, and improved color, such as a whiter color, as compared to native egg white or native whole egg and compositions made with native egg white or native whole egg. In some embodiments, the functional characteristics provided by the one or more recombinant egg-related proteins is substantially the same or better than the same functional characteristic provided by a native egg white or native egg (e.g., whole egg).

rOVD, rOVA, or rOVL may include additional sequences. An rOVD, rOVA and/or rOVL may be a non-naturally occurring variant, which may include one or more amino acid insertions, deletions, or substitutions relative to native OVD, native OVA, or native OVL sequence. Expression of rOVD, rOVA and rOVL in a host cell (i.e., a Pichia species) can lead to additional peptides to the sequences as part of post-transcriptional or post translational modifications.

In some embodiments, a recombinant protein in the substantially liquid/powdered mixture is recombinant ovomucoid (rOVD). In some embodiments, rOVD is the only recombinant egg-related protein. In some embodiments, the substantially liquid/powdered mixture comprises rOVD and one or more other recombinant egg-related proteins.

In some embodiments, preparation of the egg-related proteins mentioned above may comprise drying the proteins. In some embodiments, drying may comprise spray drying and/or lyophilization.

In some embodiments, the glycosylation pattern of rOVD is the same as the glycosylation pattern of a native chicken ovomucoid. In some embodiments, the glycosylation pattern of rOVD is different from the glycosylation pattern of a native chicken ovomucoid. In some embodiments, rOVD has no glycosylation. In some embodiments, rOVD is substantially devoid of glycosylation. In some embodiments, rOVD is highly glycosylated. In some embodiments, rOVD has reduced glycosylation. In some embodiments, rOVD comprises at least one glycosylated asparagine residue. In some embodiments, the at least one glycosylated asparagine residue comprises a single N-acetylglucosamine. In some embodiments, rOVD comprises at least three glycosylated asparagine residues. In some embodiments, rOVD lacks or is substantially devoid of N-linked mannosylation. In some embodiments, the glycosylation pattern of rOVD is the same as the glycosylation pattern of a native chicken ovomucoid. In some embodiments, the glycosylation pattern of rOVD is different from the glycosylation pattern of a native chicken ovomucoid.

In various embodiments, the rOVD has at least 80%, 85%, 90%, 92%, 95%, 96%, 97%, 98%, 99% of 100% sequence identity to one of SEQ ID NO: 1 to SEQ ID NO: 44. In some embodiments, a variant is one that confers additional features, such as reduced allergenicity. For example, an rOVD can include G162M and/or F167A (such as in SEQ ID NO: 3) relative to a wild type OVD sequence SEQ ID NO: 2 and have reduced allergenicity as compared to the wild type OVD sequence.

In various embodiments, the rOVD is present in the substantially liquid mixture at a concentration from about 0.1% to about 20% on a weight per weigh (w/w) or weight per volume (w/v) basis. In embodiments, the rOVD is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the rOVD is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, or about 20% w/w or w/v. In various embodiments, the rOVD is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, or 18% to 20% w/w or w/v.

In some cases, a composition here is a liquid or powdered composition. In some cases, the weight percent of rOVD to composition is 0.15% to 4.5% on a w/w basis. In some cases, the weight percent of rOVD to composition is at least 0.15% on a w/w basis. In some cases, the weight percent of rOVD to composition is at most 4.5% on a w/w basis. In some cases, the weight percent of rOVD to composition is 0.15% to 0.5%, 0.15% to 1%, 0.15% to 1.5%, 0.15% to 2%, 0.15% to 2.5%, 0.15% to 3%, 0.15% to 3.5%, 0.15% to 4%, 0.15% to 4.5%, 0.5% to 1%, 0.5% to 1.5%, 0.5% to 2%, 0.5% to 2.5%, 0.5% to 3%, 0.5% to 3.5%, 0.5% to 4%, 0.5% to 4.5%, 1% to 1.5%, 1% to 2%, 1% to 2.5%, 1% to 3%, 1% to 3.5%, 1% to 4%, 1% to 4.5%, 1.5% to 2%, 1.5% to 2.5%, 1.5% to 3%, 1.5% to 3.5%, 1.5% to 4%, 1.5% to 4.5%, 2% to 2.5%, 2% to 3%, 2% to 3.5%, 2% to 4%, 2% to 4.5%, 2.5% to 3%, 2.5% to 3.5%, 2.5% to 4%, 2.5% to 4.5%, 3% to 3.5%, 3% to 4%, 3% to 4.5%, 3.5% to 4%, 3.5% to 4.5%, or 4% to 4.5% on a w/w basis. In some cases, the weight percent of rOVD to composition is 0.15%, 0.5%, 1%, 1.5%, 2%, 2.5%, 3%, 3.5%, 4%, or 4.5% on a w/w basis. In some preferred embodiments, the weight percent of rOVD in a liquid composition may be from 0.2% to 4% on a w/w basis.

In various embodiments, the rOVD is present in a substantially dry mixture at a concentration of from about 20% to about 50% on a w/w basis. In various embodiments, the rOVD is present in a substantially dry mixture at a concentration of from at least about 20% on a w/w basis. In various embodiments, the rOVD is present in a substantially dry mixture at a concentration of from at most about 50% on a w/w basis. In various embodiments, the rOVD is present in a substantially dry mixture at a concentration of from about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50% on a w/w basis. In various embodiments, the rOVD is present in a substantially dry mixture at a concentration of from about 20%, about 25%, about 30%, about 35%, about 40%, about 45%, or about 50% on a w/w basis.

In some embodiments, a recombinant protein in the mixture is recombinant ovalbumin (rOVA). In some embodiments, rOVA is the only recombinant egg-related protein. In some embodiments, the substantially liquid/powdered mixture comprises rOVA and one or more other recombinant egg-related proteins. In some embodiments, rOVA comprises duck rOVA, chicken rOVA and/or ostrich rOVA.

In some embodiments, the rOVA comprises the amino acid sequence of a duck OVA.

In some embodiments, the rOVA comprises the amino acid sequence of an ostrich OVA.

In some embodiment, the rOVA has one or more N-linked glycosylation sites having mannose linked to an N-acetyl glucosamine. In various embodiments, the rOVA has one or more N-linked glycosylation sites having mannose linked to an N-acetyl glucosamine in which the N-linked glycosylation sites lack galactose. In some embodiments, the glycosylation pattern of rOVA is different from the glycosylation pattern of a native chicken ovalbumin. In some embodiments, the glycosylation pattern of rOVA comprises N-linked glycan structures such as N-acetylglucosamine units, galactose and N-linked mannose units. In some embodiments, the glycosylation pattern of rOVA comprises one or more N-linked glycosylation sites having mannose linked to an N-acetyl glucosamine. In some embodiments, the N-linked glycosylations site does not comprise, or lacks, galactose.

In various embodiments, the rOVA has at least 80%, 85%, 90%, 92%, 95%, 96%, 97%, 98%, 99% of 100% sequence identity to any one of SEQ ID NO: 45 to SEQ ID NO: 118.

In various embodiments, the rOVA is present in the substantially liquid mixture at a concentration from about 0.1% to about 40% on a weight per weigh (w/w) or weight per volume (w/v) basis. In embodiments, the rOVA is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the rOVA is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the rOVA is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v.

In various embodiments, the rOVA is present in a substantially dry mixture at a concentration of from about 10% to about 90% w/w or w/v. In various embodiments, the rOVA is present in a substantially dry mixture at a concentration of from at least about 10% w/w or w/v. In various embodiments, the rOVA is present in a substantially dry mixture at a concentration of from at most about 90% w/w or w/v. In various embodiments, the rOVA is present in a substantially dry mixture at a concentration of from about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 20% to about 25%, about 20% to about 30%, about 20% to about 40%, about 20% to about 50%, about 20% to about 60%, about 20% to about 70%, about 20% to about 80%, about 20% to about 90%, about 25% to about 30%, about 25% to about 40%, about 25% to about 50%, about 25% to about 60%, about 25% to about 70%, about 25% to about 80%, about 25% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% w/w or w/v. In various embodiments, the rOVA is present in a substantially dry mixture at a concentration of from about 10%, about 20%, about 25%, about 30%, about 40%, about 50%, about 60%, about 70%, about 80%, or about 90% w/w or w/v.

In some cases, the weight percent of rOVA to composition is 2% to 10% on a w/w basis. In some cases, the weight percent of rOVA to composition is at least 2% on a w/w basis. In some cases, the weight percent of rOVA to composition is at most 10% on a w/w basis. In some cases, the weight percent of rOVA to composition is 2% to 3%, 2% to 4%, 2% to 5%, 2% to 6%, 2% to 7%, 2% to 8%, 2% to 9%, 2% to 10%, 3% to 4%, 3% to 5%, 3% to 6%, 3% to 7%, 3% to 8%, 3% to 9%, 3% to 10%, 4% to 5%, 4% to 6%, 4% to 7%, 4% to 8%, 4% to 9%, 4% to 10%, 5% to 6%, 5% to 7%, 5% to 8%, 5% to 9%, 5% to 10%, 6% to 7%, 6% to 8%, 6% to 9%, 6% to 10%, 7% to 8%, 7% to 9%, 7% to 10%, 8% to 9%, 8% to 10%, or 9% to 10% on a w/w basis. In some cases, the weight percent of rOVA to composition is 2%, 3%, 4%, 5%, 6%, 7%, 8%, 9%, or 10% on a w/w basis. In some preferred embodiments, the weight percent of rOVA in a liquid composition may be from 2% to 9% on a w/w basis.

In some embodiments, a recombinant protein in the substantially liquid/powdered mixture is recombinant lysozyme (rOVL). In some embodiments, the substantially liquid/powdered mixture comprises rOVL as the only recombinant egg-related protein. In some embodiments, the substantially liquid/powdered mixture comprises rOVL and one or more other recombinant egg-related proteins.

In some embodiments, the glycosylation pattern of rOVL is different from the glycosylation, acetylation, or phosphorylation of a native chicken lysozyme. In some embodiments, rOVL is deglycosylated, deacetylated, or dephosphorylated.

In some embodiments, the rOVL is a recombinant chicken egg white lysozyme (cOVL) and/or a recombinant goose lysozyme (gOVL). In some embodiments, gOVL may be used as a gelling agent. In some embodiments, the substantially liquid/powdered mixture comprises gOVL as the only gelling agent. In some embodiments, the substantially liquid/powdered mixture comprises gOVL used as a gelling agent and other gelling agents. gOVL may increase favorable qualities of a food product, e.g., increased gelling and firmness to a solid or semi-solid food product or increased viscosity to a liquid/powdered food product. For example, gOVL has the unexpected effect of forming a gel in a solution without needing additional gelling agents. In some embodiments, the substantially liquid/powdered mixture comprises gOVL as a antimicrobial. A gOVL may be used to degrade or digest cell wall peptidoglycans of certain microorganisms such as bacteria to form gels. In some cases, gOVL may be able to form a gel without degrading or digesting a microbial cell wall. In some cases, a gel composition formed upon heat treatment of gOVL may not comprise any microbial impurities. In some cases, a gel composition formed upon heat treatment of a gOVL such as gOVL may not comprise any bacterial impurities. In some cases, a gel composition formed upon heat treatment of gOVL may not comprise any other gelling or binding agents. In some cases, gOVL may provide improved gelation to a composition as compared to the gelation provided by a chicken muramidase.

In various embodiments, the rOVL has at least 80%, 85%, 90%, 92%, 95%, 96%, 97%, 98%, 99% of 100% sequence identity to any one of SEQ ID NO: 119 to SEQ ID NO: 125.

In various embodiments, the rOVL is present in the substantially liquid mixture at a concentration from about 0.1% to about 40% on a weight per weigh (w/w) or weight per volume (w/v) basis. In embodiments, the rOVL is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the rOVL is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the rOVL is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v. In an embodiment, the rOVL is present in the substantially liquid mixture at a concentration of about 20% w/w or w/v.

In some embodiments, the rOVL is a recombinant chicken egg white lysozyme (cOVL). In various embodiments, the cOVL is present in the substantially liquid mixture at a concentration from about 0.1% to about 40% on a weight per weigh (w/w) or weight per volume (w/v) basis. In embodiments, the cOVL is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the cOVL is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the cOVL is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v. In an embodiment, the cOVL is present in the substantially liquid mixture at a concentration of about 20%.

In some embodiments, the rOVL is a recombinant goose lysozyme (gOVL). In various embodiments, the gOVL is present in the substantially liquid mixture at a concentration from about 0.1% to about 40% on a weight per weigh (w/w) or weight per volume (w/v) basis. In embodiments, the gOVL is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the gOVL is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the gOVL is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v. In an embodiment, the gOVL is present in the substantially liquid mixture at a concentration of about 20% w/w or w/v.

In some embodiments, the substantially liquid/powdered mixture comprises one or more egg-related proteins. In some embodiments, the one or more egg-related proteins comprise a recombinant protein. In some embodiments, the recombinant protein comprises at least one, at least two, at least three, at least four, at least five, at least six, at least seven, at least eight, at least nine, or at least ten different recombinant egg-related proteins.

In some embodiments, the recombinant protein is a recombinant ovalbumin (rOVA), recombinant ovomucoid (rOVD), a recombinant egg white lysozyme (rOVL), or a combination thereof. In some embodiments, the recombinant protein comprises rOVA alone, rOVD alone, rOVL alone, a combination of rOVA and rOVD, a combination of rOVA and rOVL, rOVA, a combination of rOVA and rOVL, or a combination of rOVD and rOVL. In some embodiments, the recombinant protein consists of a combination of rOVD and rOVA.

In various embodiments, the combination of two or more of rOVA, rOVD, rOVL is present in the substantially liquid/powdered mixture at a total concentration from about 0.1% to about 40% on a weight per weigh (w/w) or weight per volume (w/v) basis. In embodiments, the combination of two or more of rOVA, rOVD, rOVL is present in the substantially liquid/powdered mixture at a total concentration of about 0.10%, 0.20%, 0.30%, 0.40%, 0.50%, 0.60%, 0.70%, 0.80%, 0.90%, or about 1.00% w/w or w/v. In some embodiments, the combination of two or more of rOVA, rOVD, rOVL is present in the substantially liquid/powdered mixture at a total concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the combination of two or more of rOVA, rOVD, rOVL is present in the substantially liquid/powdered mixture at a total concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v.

In some embodiments, the substantially liquid/powdered mixture comprises rOVA and rOVD, with a ratio of rOVA to rOVD, on a weight per weight (w/w) or weight per volume (w/v) basis, is between about 1:11 and about 11:1. In some embodiments, the substantially liquid/powdered mixture comprises rOVA and rOVD in a ratio of, on a weight per weight (w/w) or weight per volume (w/v) basis, of about 1:1, about 1:2, about 1:3, about 1:4, about 1:5, about 1:6, about 1:7, about 1:8, about 1:9, about 1:10, about 1:11, about 2:1, about 2:3, about 2:5, about 2:7, about 2:9, about 2:11, about 3:1, about 3:2, about 3:4, about 3:5, about 3:7, about 3:8, about 3:10, about 3:11, about 4:1, about 4:3, about 4:5, about 4:7, about 4:9, about 4:11, about 5:1, about 5:2, about 5:3, about 5:4, about 5:6, about 5:7, about 5:8, about 5:9, about 5:11, about 6:1, about 6:5, about 6:7, about 6:11, about 7:1, about 7:2, about 7:3, about 7:4, about 7:5, about 7:6, about 7:8, about 7:9, about 7:10, about 7:11, about 8:1, about 8:3, about 8:5, about 8:7, about 8:9, about 8:11, about 9:1, about 9:2, about 9:4, about 9:5, about 9:7, about 9:8, about 9:10, about 9:11, about 10:1, about 10:3, about 10:7, about 10:9, about 10:11, about 11:1, about 11:2, about 11:3, about 11:4, about 11:5, about 11:6, about 11:7, about 11:8, about 11:9, or about 11:10.

In some embodiments, the substantially liquid/powdered mixture comprises rOVA and rOVD, with a ratio of rOVA to rOVD, on a weight per weight (w/w) or weight per volume (w/v) basis, is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1 on a weight per weight (w/w) or weight per volume (w/v) basis. In some cases, a weight ratio of rOVD and rOVA is from about 1:50 to about 2:1 on a weight per weight (w/w) or weight per volume (w/v) basis. In some cases, a weight ratio of rOVD and rOVA is from 1:50 to 1:40, 1:50 to 1:30, 1:50 to 1:10, 1:50 to 1:5, 1:50 to 1:3, 1:50 to 1:2, 1:50 to 1:1 on a weight per weight (w/w) or weight per volume (w/v) basis. In some cases, a weight ratio of rOVD and rOVA is from 1:30 to 1:10, 1:30 to 1:5, 1:30 to 1:3, 1:30 to 1:2, 1:30 to 1:1 on a weight per weight (w/w) or weight per volume (w/v) basis.

In some embodiments, the substantially liquid/powdered mixture comprises rOVA and any other protein described herein. In some embodiments, the substantially liquid/powdered mixture comprises rOVA and any other protein described herein on a weight per weight (w/w) or weight per volume (w/v) basis, is between about 1:11 and about 11:1. In some embodiments, the substantially liquid/powdered mixture comprises rOVA and any other protein described herein in a ratio of, on a weight per weight (w/w) or weight per volume (w/v) basis, of about 1:1, about 1:2, about 1:3, about 1:4, about 1:5, about 1:6, about 1:7, about 1:8, about 1:9, about 1:10, about 1:11, about 2:1, about 2:3, about 2:5, about 2:7, about 2:9, about 2:11, about 3:1, about 3:2, about 3:4, about 3:5, about 3:7, about 3:8, about 3:10, about 3:11, about 4:1, about 4:3, about 4:5, about 4:7, about 4:9, about 4:11, about 5:1, about 5:2, about 5:3, about 5:4, about 5:6, about 5:7, about 5:8, about 5:9, about 5:11, about 6:1, about 6:5, about 6:7, about 6:11, about 7:1, about 7:2, about 7:3, about 7:4, about 7:5, about 7:6, about 7:8, about 7:9, about 7:10, about 7:11, about 8:1, about 8:3, about 8:5, about 8:7, about 8:9, about 8:11, about 9:1, about 9:2, about 9:4, about 9:5, about 9:7, about 9:8, about 9:10, about 9:11, about 10:1, about 10:3, about 10:7, about 10:9, about 10:11, about 11:1, about 11:2, about 11:3, about 11:4, about 11:5, about 11:6, about 11:7, about 11:8, about 11:9, or about 11:10.

In some embodiments, the substantially liquid/powdered mixture comprises rOVD and any other protein described herein. In some embodiments, the substantially liquid/powdered mixture comprises rOVD and any other protein described herein on a weight per weight (w/w) or weight per volume (w/v) basis, is between about 1:11 and about 11:1. In some embodiments, the substantially liquid/powdered mixture comprises rOVD and any other protein described herein in a ratio of, on a weight per weight (w/w) or weight per volume (w/v) basis, of about 1:1, about 1:2, about 1:3, about 1:4, about 1:5, about 1:6, about 1:7, about 1:8, about 1:9, about 1:10, about 1:11, about 2:1, about 2:3, about 2:5, about 2:7, about 2:9, about 2:11, about 3:1, about 3:2, about 3:4, about 3:5, about 3:7, about 3:8, about 3:10, about 3:11, about 4:1, about 4:3, about 4:5, about 4:7, about 4:9, about 4:11, about 5:1, about 5:2, about 5:3, about 5:4, about 5:6, about 5:7, about 5:8, about 5:9, about 5:11, about 6:1, about 6:5, about 6:7, about 6:11, about 7:1, about 7:2, about 7:3, about 7:4, about 7:5, about 7:6, about 7:8, about 7:9, about 7:10, about 7:11, about 8:1, about 8:3, about 8:5, about 8:7, about 8:9, about 8:11, about 9:1, about 9:2, about 9:4, about 9:5, about 9:7, about 9:8, about 9:10, about 9:11, about 10:1, about 10:3, about 10:7, about 10:9, about 10:11, about 11:1, about 11:2, about 11:3, about 11:4, about 11:5, about 11:6, about 11:7, about 11:8, about 11:9, or about 11:10.

In some embodiments, a substantially liquid/powdered mixture comprises a protein component, e.g., other than the egg-related protein mentioned above. In some embodiments, a substantially liquid/powdered mixture comprises an egg-related protein (e.g., a recombinant egg-related protein) and a protein component.

In some embodiments, the protein component comprises one or more types of protein. In some embodiments, the protein component comprises at least one, at least two, at least three, at least four, at least five, at least six, at least seven, at least eight, at least nine, or at least ten different types of protein.

In some embodiments, the substantially liquid/powdered mixture comprises protein component that is a plant-based protein. In some embodiments, the substantially liquid/powdered mixture comprises a protein component that comprises a plant protein and one or more recombinant proteins. In some embodiments, the substantially liquid/powdered mixture comprises a protein component that comprises chickpea protein, one or more other plant proteins, and one or more recombinant proteins.

In some embodiments, the protein component is present in the substantially liquid mixture at a concentration from about 0.1% to about 30% on a weight per weight or weight per volume basis. In various embodiments, the protein component is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the protein component is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the protein component is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v. In an embodiment, the protein component is present in the substantially liquid mixture at a concentration of about 20%.

The protein content (as measured by w/w or w/w) will be greater in a substantially dry composition, i.e., which lacks water. In some embodiments, the protein component is present in the substantially dry mixture at a concentration 1% to 95% w/w or w/v. In some embodiments, the protein component is present in the substantially dry mixture at a concentration at least 1% w/w or w/v. In some embodiments, the protein component is present in the substantially dry mixture at a concentration at most 95% w/w or w/v. In some embodiments, the protein component is present in the substantially dry mixture at a concentration 1% to 5%, 1% to 10%, 1% to 20%, 1% to 30%, 1% to 40%, 1% to 50%, 1% to 60%, 1% to 70%, 1% to 80%, 1% to 95%, 5% to 10%, 5% to 20%, 5% to 30%, 5% to 40%, 5% to 50%, 5% to 60%, 5% to 70%, 5% to 80%, 5% to 95%, 10% to 20%, 10% to 30%, 10% to 40%, 10% to 50%, 10% to 60%, 10% to 70%, 10% to 80%, 10% to 95%, 20% to 30%, 20% to 40%, 20% to 50%, 20% to 60%, 20% to 70%, 20% to 80%, 20% to 95%, 30% to 40%, 30% to 50%, 30% to 60%, 30% to 70%, 30% to 80%, 30% to 95%, 40% to 50%, 40% to 60%, 40% to 70%, 40% to 80%, 40% to 95%, 50% to 60%, 50% to 70%, 50% to 80%, 50% to 95%, or 60% to 70%, 60% to 80%, 60% to 95%, 70% to 80%, 70% to 95%, 80% to 95% w/w or w/v. In some embodiments, the protein component is present in the substantially dry mixture at a concentration 1%, 5%, 10%, 20%, 30%, 40%, 50%, 60%, 70%, 80%, or 95% w/w or w/v.

As used herein, the term “plant protein” is used synonymously with the term “plant-based protein.” In some embodiments, the plant protein comprises one or more types of proteins. In some embodiments, the plant protein comprises one or more of chickpea protein, pumpkin protein, sunflower protein, mungbean protein, chia protein, sesame seed protein, flaxseed protein, tara protein, rice protein, fava bean protein, mushroom protein, hemp protein, and pea protein. In some embodiments, the plant protein comprises chickpea protein. In some embodiments, the plant protein is chickpea and one or more other plant proteins.

In some embodiments, the protein component comprises one or more plant proteins which are present in the substantially liquid mixture at a concentration from about 0.1% to about 30% on a weight per weight or weight per volume basis. In various embodiments, the protein component comprising one or more plant proteins is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the protein component comprising one or more plant proteins is present in the substantially liquid mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the protein component comprising one or more plant proteins is present in the substantially liquid mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v. In an embodiment, the protein component comprising one or more plant proteins is present in the substantially liquid mixture at a concentration of about 20%.

In some embodiments, the substantially liquid/powdered mixture comprises a dietary fiber-providing component. A dietary fiber-providing component may improve the nutritional value of the composition, improve gelation properties, and increase the composition's water holding capacity. In some embodiments, the dietary fiber-providing component comprises one type of fiber, two types of fiber, or more than two types of fiber. In some embodiments, the dietary fiber-providing component comprises an insoluble fiber. In some embodiments, the dietary fiber-providing component comprises a soluble fiber.

In some embodiments, the dietary fiber-providing component is present in the substantially liquid mixture at a concentration of about 0.1% to about 10% on a weight per weight or weight per volume basis. In various embodiments, the dietary fiber-providing component is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the dietary fiber-providing component is present in the substantially liquid mixture at a concentration of about 1%, 2%, 3%, 4%, 5%, 6%, 7%, 8%, 9%, or about 10% w/w or w/v. In various embodiments, the dietary fiber-providing component is present in the substantially liquid mixture at a concentration of from about 1% to 2%, 1% to 3%, 2% to 3%, 2% to 5%, 3% to 4%, 4% to 5%, 4% to 7%, 5% to 6%, 6% to 7%, 6% to 9%, 7% to 8%, 8% to 9%, 8% to 10%, or 9% to 10% w/w or w/v.

In some embodiments, the dietary fiber-providing component comprises one or more plant fibers. The plant fiber may be selected from Psyllium husk, bamboo fiber, oat fiber, carrot fiber, flaxseed, chia seed, wheat fiber, pea fiber, potato fiber, apple fiber, citrus fiber, accacia fiber, cellulose fiber, inulin, lignin, mucilage, pectin, polydextrose, resistant starch, wheat dextrin, wheat bran, alginates, raffinose, legumes, oats, rye, barley, fruit fibers, root tuber, beta-glucans, lignin, or any combination thereof. In some embodiments, the fiber-providing component is Psyllium, e.g., Psyllium husk fiber.

In some embodiments, the plant fiber is present in the substantially liquid mixture at a concentration of about 0.1% to about 10% on a weight per weight or weight per volume basis. In various embodiments, the plant fiber is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the plant fiber is present in the substantially liquid mixture at a concentration of about 1%, 2%, 3%, 4%, 5%, 6%, 7%, 8%, 9%, or about 10% w/w or w/v. In various embodiments, the plant fiber is present in the substantially liquid mixture at a concentration of from about 1% to 2%, 1% to 3%, 2% to 3%, 2% to 5%, 3% to 4%, 4% to 5%, 4% to 7%, 5% to 6%, 6% to 7%, 6% to 9%, 7% to 8%, 8% to 9%, 8% to 10%, or 9% to 10% w/w or w/v.

In some embodiments, the fiber-providing component is Psyllium husk fiber. In some embodiments, the Psyllium husk fiber is present in the substantially liquid mixture at a concentration of about 0.1% to about 5% on a weight per weight or weight per volume basis. In various embodiments, the Psyllium husk fiber is present in the substantially liquid mixture at a concentration of about 0.20%, 0.30%, 0.40%, 0.50%, 0.60%, 0.70%, 0.80%, 0.90%, or 1.00% w/w or w/v. In some embodiments, the Psyllium husk fiber is present in the substantially liquid mixture at a concentration of about 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, or 5% w/w or w/v. In various embodiments, the Psyllium husk fiber is present in the substantially liquid mixture at a concentration of from about 1% to 2%, 1% to 3%, 2% to 3%, 2% to 5%, 3% to 4%, or 4% to 5% w/w or w/v. In some embodiments, the Psyllium husk fiber is present in the substantially liquid mixture at a concentration of about 0.7% w/w or w/v.

In some embodiments, the substantially liquid/powdered mixture comprises a starch-providing component. In some embodiments, the substantially liquid/powdered mixture comprises a starch-providing component comprising polysaccharides. In some embodiments, the substantially liquid/powdered mixture comprises a starch-providing component comprising polysaccharides has glucose monomers joined via α-1,4 linkages.

A starch-providing component may be any food, food item or food ingredient that contains one or more forms of starch. A starch component may provide such functions as increasing the viscosity, improving the body, and improving the mouthfeel of a composition. In some embodiments, the starch-providing component is a polysaccharide. In some embodiments, the polysaccharide comprises glucose monomers. In various embodiments, the polysaccharide comprises glucose monomers joined via α-1,4 linkages. In some embodiments, the glucose monomers joined via α-1,4 linkages comprise amylose and/or amylopectin. In some embodiments, the starch-providing component is potato starch, tapioca starch, corn, arrowroot starch, tapioca starch, and/or rice syrup.

In some embodiments, the starch-providing component is present in the substantially liquid mixture at a concentration of about 0.1% to about 20% on a weight per weight or weight per volume basis. In some embodiments, the starch-providing component is present in the substantially liquid mixture at a concentration of about 0.5%, about 1%, about 2%, about 3%, about 4%, about 5%, about 10%, about 15%, or about 20% on a w/w or w/v basis. In some embodiments, the starch-providing component is present in the substantially liquid mixture at a concentration of 0.1% to 20% on a w/w or w/v basis. In some embodiments, the starch-providing component is present in the substantially liquid mixture at a concentration of 0.1% to 0.5%, 0.1% to 1%, 0.1% to 2%, 0.1% to 3%, 0.1% to 4%, 0.1% to 5%, 0.1% to 10%, 0.1% to 15%, 0.1% to 20%, 0.5% to 1%, 0.5% to 2%, 0.5% to 3%, 0.5% to 4%, 0.5% to 5%, 0.5% to 10%, 0.5% to 15%, 0.5% to 20%, 1% to 2%, 1% to 3%, 1% to 4%, 1% to 5%, 1% to 10%, 1% to 15%, 1% to 20%, 2% to 3%, 2% to 4%, 2% to 5%, 2% to 10%, 2% to 15%, 2% to 20%, 3% to 4%, 3% to 5%, 3% to 10%, 3% to 15%, 3% to 20%, 4% to 5%, 4% to 10%, 4% to 15%, 4% to 20%, 5% to 10%, 5% to 15%, 5% to 20%, 10% to 15%, 10% to 20%, or 15% to 20% on a w/w or w/v basis. In some embodiments, the starch-providing component is present in the substantially liquid mixture at a concentration of 0.1%, 0.5%, 1%, 2%, 3%, 4%, 5%, 10%, 15%, or 20% on a w/w or w/v basis. In some embodiments, the starch-providing component is present in the substantially liquid mixture at a concentration of at least 0.1%, 0.5%, 1%, 2%, 3%, 4%, 5%, 10%, or 15% on a w/w or w/v basis. In some embodiments, the starch-providing component is present in the substantially liquid mixture at a concentration of at most 0.5%, 1%, 2%, 3%, 4%, 5%, 10%, 15%, or 20% on a w/w or w/v basis.

In some embodiments, the substantially liquid mixture comprises a gelation agent. Gelation agents in the composition may increase the composition's gelation properties, water holding functionality, mouthfeel and body. As used herein, “gelation agent” is used synonymously with “gelling agent.” In some embodiments, the gelation agent is a polysaccharide and/or a recombinant protein. In some embodiments, the one or more gelation agents comprises one or more polysaccharide-based hydrocolloids or protein-based hydrocolloids. In some embodiments, the gelation agent is beta-glucan, hydroxypropyl methylcellulose, carboxy methylcellulose, methylcellulose, carrageenan, locust bean gum, sodium alginate, xanthan gum, gellan gum (e.g., high acyl gellan gum and low acyl gellan gum), tara gum, agar, lecithin, transglutaminase or konjac gum. In some embodiments, the gelation agent is a recombinant protein, e.g., recombinant goose lysozyme (gOVL). In some embodiments, the gelation agent is combination of a polysaccharide and a recombinant protein.

In some embodiments, the gelation agent is present in the substantially liquid mixture at a concentration of about 0.01% to about 5% on a weight per weight or weight per volume basis. In some embodiments, the gelation agent is present in the substantially liquid mixture at a concentration of about 0.01%, 0.02%, 0.03%, 0.04%, 0.05%, 0.06%, 0.07%, 0.08%, 0.09%, or 0.10% w/w or w/v. In various embodiments, the gelation agent is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the gelation agent is present in the substantially liquid mixture at a concentration of about 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, or 5% w/w or w/v. In various embodiments, the gelation agent is present in the substantially liquid mixture at a concentration of from about 1% to 2%, 1% to 3%, 2% to 3%, 2% to 5%, 3% to 4%, 4% to 5% w/w or w/v. In some embodiments, the gelation agent is present in the substantially liquid mixture at a concentration of about 0.5% w/w or w/v.

In some embodiments, the gelation agent is a beta-glucan. Beta-glucan may be useful in improving gelation properties. In some cases, the beta-glucan is one or more of a bacterial beta-glucan, barley beta-glucan, Betafectin/TH-glucan, botryosphaeran, callose, carboxymethylpachymaran, cereal beta-glucan, cerevan, chitin-glucan, chrysolaminarin, coriolan, curdlan, epiglucan, fungal beta-glucan, grifolan, krestin, laminaran/laminarin, latiglucan, lentinan, leucosin, lichenan/lichenin, mycolaminarin, oat beta-glucan, pachymaran/pachyman, paramylon, pendulan, pestalotan, phycarine, pleuran, polycan, polysaccharide-glucan, pustulan, scleroglucan/sclero-beta-glucan, sclerotinan/sclerotan, tylopilan, yeast beta-glucan, yestimun, and zymosan.

In some embodiments, the gelation agent is transglutaminase (TG). TG may be useful in improving gelation properties.

In some cases, TG improves gelation properties of ovalbumin, including recombinant ovalbumin, e.g., an rOVA comprising the amino acid sequence of a duck OVA or an rOVA comprising the amino acid sequence of an ostrich OVA.

In some embodiments, the substantially liquid/powdered mixture comprises TG and recombinant lysozyme (rOVL). In some embodiments, the substantially liquid/powdered mixture comprises TG and another gelation agent, as disclosed herein. In some embodiments, TG is used with rOVL and another gelation agent, as disclosed herein. In some embodiments, the substantially liquid/powdered mixture comprises TG in the absence of rOVL. In some embodiments, the substantially liquid/powdered mixture comprises TG in the absence of another gelation agent. In some embodiments, the substantially liquid/powdered mixture comprises TG in the absence of a rOVL and another gelation agent.

In some embodiments, the substantially liquid/powdered mixture comprises transglutaminase as a gelation agent and comprises mung bean protein as a protein component.

In some embodiments, transglutaminase is present in the substantially liquid mixture at a concentration of about 0.001% to about 5% on a w/w or w/v basis. In some embodiments, transglutaminase is present in the substantially liquid mixture at a concentration of about 0.001% to about 0.01%, about 0.001% to about 0.1%, about 0.001% to about 0.5%, about 0.001% to about 1%, about 0.001% to about 1.5%, about 0.001% to about 2%, about 0.001% to about 3%, about 0.001% to about 4%, about 0.001% to about 5%, about 0.01% to about 0.1%, about 0.01% to about 0.5%, about 0.01% to about 1%, about 0.01% to about 1.5%, about 0.01% to about 2%, about 0.01% to about 3%, about 0.01% to about 4%, about 0.01% to about 5%, about 0.1% to about 0.5%, about 0.1% to about 1%, about 0.1% to about 1.5%, about 0.1% to about 2%, about 0.1% to about 3%, about 0.1% to about 4%, about 0.1% to about 5%, about 0.5% to about 1%, about 0.5% to about 1.5%, about 0.5% to about 2%, about 0.5% to about 3%, about 0.5% to about 4%, about 0.5% to about 5%, about 1% to about 1.5%, about 1% to about 2%, about 1% to about 3%, about 1% to about 4%, about 1% to about 5%, about 1.5% to about 2%, about 1.5% to about 3%, about 1.5% to about 4%, about 1.5% to about 5%, about 2% to about 3%, about 2% to about 4%, about 2% to about 5%, about 3% to about 4%, about 3% to about 5%, or about 4% to about 5% on a w/w or w/v basis. In some embodiments, transglutaminase is present in the substantially liquid mixture at a concentration of about 0.001%, about 0.01%, about 0.1%, about 0.5%, about 1%, about 1.5%, about 2%, about 3%, about 4%, or about 5% on a w/w or w/v basis. In some embodiments, transglutaminase is present in the substantially liquid mixture at a concentration of at least about 0.001%, about 0.01%, about 0.1%, about 0.5%, about 1%, about 1.5%, about 2%, about 3%, or about 4% on a w/w or w/v basis. In some embodiments, transglutaminase is present in the substantially liquid mixture at a concentration of at most about 0.01%, about 0.1%, about 0.5%, about 1%, about 1.5%, about 2%, about 3%, about 4%, or about 5% on a w/w or w/v basis.

In some embodiments, the dietary fiber-providing component also serves as a gelation agent. As non-limiting examples, Psyllium husk, oat fiber, chia seed, inulin, and pectin can act as both a gelation agent and as a dietary fiber-providing component in substantially liquid/powdered mixture.

In some embodiments, the substantially liquid/powdered mixture comprises a flavoring agent. In some embodiments, the flavoring agent comprises water and oil-based flavors in both liquid/powdered and powder forms, yeast, rock salt, rock salts (such as kala namak), and/or amino acids (i.e., cysteine, cystine, and methionine). Amino acids may be used to impart a sulfur-like flavor to the substantially liquid/powdered mixture. In some embodiments, the flavoring agent comprises a salt. The inclusion of a salt within the composition may be useful in increasing ionic strength, gelation, and taste. In some embodiments, the salt comprises a Na⁺, Ca⁺², K⁺, or Mg⁺² cation. In some embodiments, the salt comprises a lactate (e.g., calcium lactate), Cl⁻, gluconate, or propionate anion. In some embodiments, the salt comprises acid salts, alkali salts, organic salts, inorganic salts, phosphates, chloride salts, sodium salts, sodium chloride, potassium salts, potassium chloride, magnesium salts, magnesium chloride, magnesium perchlorate, calcium salts, calcium chloride, ammonium chloride, iron salts, iron chlorides, zinc salts, lactate salts, gluconate salts, propionate salts, rock salts (such as kala namak), coarse salt and/or zinc chloride. In some embodiments, the salt comprises calcium lactate. In some embodiments, the substantially liquid/powdered mixture comprises rock salts (such as kala namak) and one or more of any salt mentioned herein. In some embodiments, the salt comprises one or more salts mentioned herein.

In some embodiments, the flavoring agent is present in the substantially liquid mixture in a concentration of between about 0.001% to about 5% on a weight by weight or weight by volume basis. In embodiments, the flavoring agent is present in the substantially liquid mixture at a concentration of about 0.001%, 0.002%, 0.003%, 0.004%, 0.005%, 0.006%, 0.007%, 0.008%, 0.009%, or about 0.01% w/w or w/v. In embodiments, the flavoring agent is present in the substantially liquid mixture at a concentration of about 0.01%, 0.02%, 0.03%, 0.04%, 0.05%, 0.06%, 0.07%, 0.08%, 0.09%, or about 0.1% w/w or w/v. In embodiments, the flavoring agent is present in the substantially liquid mixture at a concentration of about 0.1%, 0.2%, 0.3%, 0.4%, 0.5%, 0.6%, 0.7%, 0.8%, 0.9%, or about 1% w/w or w/v. In embodiments, the flavoring agent is present in the substantially liquid mixture at a concentration of about 1.2%, 1.4%, 1.6%, 1.8%, 2%, 2.2%, 2.4%, 2.6%, 2.8%, or about 3% w/w or w/v. In some embodiments, the flavoring agent is present in the substantially liquid mixture at a concentration of about 3.2%, 3.4%, 3.6%, 3.8%, 4%, 4.2%, 4.4%, 4.6%, 4.8% or about 5%. In various embodiments, the flavoring agent is present in the substantially liquid mixture at a concentration of from about 1% to 2%, 1% to 3%, 1% to 4%, 1% to 5%, 2% to 3%, 2% to 4%, 2% to 5%, 3% to 4%, 3% to 5%, or 4% to 5% w/w or w/v.

In some embodiments, the flavoring agent comprises rock salts (such as kala namak). Rock salts (such as kala namak) is a kiln-fired rock salt used in South Asia with a sulphurous, pungent-smell. It is also known as “Himalayan black salt”, Sulemani namak, bire noon, bit lobon, kala loon, or pada loon and manufactured from the salts mined in the regions surrounding the Himalayas. Rock salts (such as kala namak) is composed largely of sodium chloride with several other components lending the salt its color and smell. The smell is mainly due to its sulfur content. Any salt that provides a sulfurous smell and/or taste may be used in a substantially liquid/powdered mixture of the present disclosure.

In some embodiments, rock salts (such as kala namak) is present in the substantially liquid mixture at a concentration of between 0.1 to 2% on a weight per weight or weight per volume basis. In embodiments, the rock salts (such as kala namak) is present in the substantially liquid mixture at a concentration of about 0.1%, 0.2%, 0.3%, 0.4%, 0.5%, 0.6%, 0.7%, 0.8%, 0.9%, or about 1% w/w or w/v. In embodiments, the rock salts (such as kala namak) is present in the substantially liquid mixture at a concentration of about 1.2%, 1.4%, 1.6%, 1.8%, 2%,

In some embodiments, the substantially liquid/powdered mixture comprises a flavoring agent that is a salt, with the salt also acting as a crosslinking agent. Thus, the salt may enhance flavor of a substantially liquid/powdered mixture and the salt may act as a crosslinking agent or the salt may act as a crosslinking agent without substantially enhancing flavor of the substantially liquid/powdered mixture. In this later case, even though the salt is categorized as a flavoring agent, its primary function is to act as a crosslinking agent. A salt acting as a crosslinking agent may be a monovalent or divalent metal cation or an anion. A monovalent or divalent metal cation may be a monovalent or divalent alkali metal ion or alkali earth metal ion. In some embodiments, a crosslinking agent is a cation. In some embodiments, cation is a Na⁺, Ca⁺², K⁺, or Mg⁺² cation. In some embodiments, the crosslinking agent comprises an anions. In some embodiments, the crosslinking agent comprising an anion is selected from lactate (e.g., calcium lactate), Cl⁻, gluconate, or propionate anions.

In some embodiments, the substantially liquid/powdered mixture comprises a lipid component. A lipid component may be useful in providing the composition with a creamy texture and an improved mouthfeel. In some embodiments, the lipid component comprises one or more triglycerides. In some embodiments, the lipid component comprises unsaturated fats. In some embodiments, the lipid component comprises saturated fats. In some embodiments, the lipid component comprises an oil. In some embodiments, the oil comprises canola oil, sunflower oil, safflower oil, olive oil, coconut oil, palm oil, and a combination thereof. In some embodiments, the lipid component comprises coconut oil or palm oil.

In some embodiments, the lipid component is present in the substantially liquid mixture in a concentration of between about 0.1% to about 30% on a weight by weight or weight by volume basis. In embodiments, the lipid component is present in the substantially liquid mixture at a concentration of about 0.1%, 0.2%, 0.3%, 0.4%, 0.5%, 0.6%, 0.7%, 0.8%, 0.9%, or about 1% w/w or w/v. In embodiments, the lipid component is present in the substantially liquid mixture at a concentration of about 1%, 2%, 3%, 4%, 5%, 6%, 7%, 8%, 9%, or about 10% w/w or w/v. In embodiments, the lipid component is present in the substantially liquid mixture at a concentration of about 10%, 11%, 12%, 13%, 14%, 15%, 16%, 17%, 18%, 19%, 20%, 21%, 22%, 23%, 24%, 25%, 26%, 27%, 28%, 29%, or about 30% w/w or w/v. In embodiments, the lipid component is present in the substantially liquid mixture at a concentration from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, or 28% to 30% w/w or w/v.

In various embodiments, the substantially liquid mixture comprises water. In some embodiments, the water will be present in the substantially liquid mixture at a concentration of about 15%, about 20%, about 25%, about 30%, about 35%, about 40%, about 45%, about 50%, about 55%, about 60%, about 65%, about 70%, about 75%, about 80%, about 85%, about 90%, or about 95% on a w/w or w/v basis. In some embodiments, the water will be present in the substantially liquid mixture at a concentration from about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 95%, about 55% to about 90%, about 55% to about 85%, about 55% to about 80%, about 55% to about 75%, about 55% to about 70%, about 55% to about 65%, about 55% to about 60%, about 60% to about 95%, about 60% to about 90%, about 60% to about 85%, about 60% to about 80%, about 60% to about 75%, about 60% to about 70%, about 60% to about 65%, about 65% to about 95%, about 65% to about 90%, about 65% to about 85%, about 65% to about 80%, about 65% to about 75%, about 65% to about 70%, about 70% to about 95%, about 70% to about 90%, about 70% to about 85%, about 70% to about 80%, about 70% to about 75%, about 75% to about 95%, about 75% to about 90%, about 75% to about 85%, about 75% to about 80%, about 80% to about 95%, about 80% to about 90%, about 80% to about 85%, about 85% to about 95%, about 85% to about 90%, or about 90% to about 95% on a w/w or w/v basis.

Further Components of a Composition and/or a Substantially Liquid/Powdered Mixture

In some embodiments, the composition and/or the substantially liquid/powdered mixture further comprises a flour. Flour may provide increased viscosity and/or gelation. In some embodiments, flour comprises chickpea flour, rice flour, corn flour, Psyllium, or combinations thereof. In some embodiments, flour is present in the composition at a concentration of about 0.1%, about 1%, about 5%, about 10%, about 15%, or about 20%.

In some embodiments, the composition and/or the substantially liquid/powdered mixture further comprises an emulsifier. A food emulsifier, also called an emulgent, is a surface-active agent that acts as a border between two immiscible liquid such as oil and water, allowing them to be blended into stable emulsions. Emulsifiers also reduce stickiness, control crystallization and prevent separation. Emulsifiers often create a smooth texture, prevent separation and extend shelf life for a food product. Commonly used emulsifiers in modern food production include mustard, soy, sunflower lecithin, and egg lecithin, mono- and diglycerides, polysorbates, carrageenan, guar gum and canola oil. In some embodiments, the substantially liquid/powdered mixture comprises an emulsifier (i.e. mono- and diglycerides, glycerol monolaurate, ethoxylated monoglyceride, diacetyl tartaric acid esters of monoglyceride, succinylated monoglyceride, calcium stearoyl-2-lactylate, sodium stearoyl-2-lactylate, propylene glycol esters, sorbitan esters, polysorbate 60, polysorbate 65, polysorbate 80, sucrose esters, and lecithin). In some embodiments, the emulsifier comprises lecithin. In some embodiments, emulsifier is present in the composition at a concentration of about 0.01%, about 0.05%, about 0.1%, about 0.5%, about 1%, about 2%, about 3%, about 4%, or about 5%.

In some embodiments the composition and/or the substantially liquid/powdered mixture further comprises a leavening agent. In some embodiments, the leavening agent comprises baking powder. In some embodiments, the leavening agent comprises yeast or baking soda. In some embodiments, leavening agent is present in the composition in a concentration of about 0.01%, about 0.05%, about 0.1%, about 0.5%, about 1%, about 2%, about 3%, about 4%, or about 5%.

In some embodiments, the composition and/or the substantially liquid/powdered mixture further a syrup component. In some embodiments, the syrup component comprises honey, plant-derived syrups, high fructose corn syrup, high maltose corn syrup, corn syrup (e.g. glucose-free corn syrup), simple syrup (e.g., comprising sucrose), sweet potato syrup, tapioca syrup, maple syrup, agave syrup, cane syrup, golden syrup, and brown rice syrup, or a combination thereof. In some embodiments, the syrup component is present in the composition and/or the substantially liquid/powdered mixture at a concentration of about 0.1%, about 0.2%, about 0.3%, about 0.4%, about 0.5%, about 0.6%, about 0.7%, about 0.8%, about 0.9%, about 1%, about 1.5%, about 2%, about 3%, about 4%, about 5%, or about 6% on a w/w or w/v basis. In some embodiments, the syrup component is present in the composition and/or the substantially liquid/powdered mixture at a concentration from about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.1% to about 1.5%, about 0.1% to about 2%, about 0.1% to about 3%, about 0.1% to about 4%, about 0.1% to about 5%, about 0.1% to about 6%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.2% to about 1.5%, about 0.2% to about 2%, about 0.2% to about 3%, about 0.2% to about 4%, about 0.2% to about 5%, about 0.2% to about 6%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.3% to about 1.5%, about 0.3% to about 2%, about 0.3% to about 3%, about 0.3% to about 4%, about 0.3% to about 5%, about 0.3% to about 6%, about 0.4% to about 0.5%, about 0.4% to about 0.6%, about 0.4% to about 0.7%, about 0.4% to about 0.8%, about 0.4% to about 0.9%, about 0.4% to about 1%, about 0.4% to about 1.5%, about 0.4% to about 2%, about 0.4% to about 3%, about 0.4% to about 4%, about 0.4% to about 5%, about 0.4% to about 6%, about 0.5% to about 0.6%, about 0.5% to about 0.7%, about 0.5% to about 0.8%, about 0.5% to about 0.9%, about 0.5% to about 1%, about 0.5% to about 1.5%, about 0.5% to about 2%, about 0.5% to about 3%, about 0.5% to about 4%, about 0.5% to about 5%, about 0.5% to about 6%, about 0.6% to about 0.7%, about 0.6% to about 0.8%, about 0.6% to about 0.9%, about 0.6% to about 1%, about 0.6% to about 1.5%, about 0.6% to about 2%, about 0.6% to about 3%, about 0.6% to about 4%, about 0.6% to about 5%, about 0.6% to about 6%, about 0.7% to about 0.8%, about 0.7% to about 0.9%, about 0.7% to about 1%, about 0.7% to about 1.5%, about 0.7% to about 2%, about 0.7% to about 3%, about 0.7% to about 4%, about 0.7% to about 5%, about 0.7% to about 6%, about 0.8% to about 0.9%, about 0.8% to about 1%, about 0.8% to about 1.5%, about 0.8% to about 2%, about 0.8% to about 3%, about 0.8% to about 4%, about 0.8% to about 5%, about 0.8% to about 6%, about 0.9% to about 1%, about 0.9% to about 1.5%, about 0.9% to about 2%, about 0.9% to about 3%, about 0.9% to about 4%, about 0.9% to about 5%, about 0.9% to about 6%, about 1% to about 1.5%, about 1% to about 2%, about 1% to about 3%, about 1% to about 4%, about 1% to about 5%, about 1% to about 6%, about 1.5% to about 2%, about 1.5% to about 3%, about 1.5% to about 4%, about 1.5% to about 5%, about 1.5% to about 6%, about 2% to about 3%, about 2% to about 4%, about 2% to about 5%, about 2% to about 6%, about 3% to about 4%, about 3% to about 5%, about 3% to about 6%, about 4% to about 5%, about 4% to about 6%, or about 5% to about 6% on a w/w or w/v basis.

In some embodiments, the composition and/or the substantially liquid/powdered mixture is substantially devoid of cholesterol.

In some various embodiments, the composition exhibits, upon cooking, a gelling capability that is equal to or exceeds the gelling capability of one or both of natural egg white or a whole egg.

In embodiments, the composition has a shelf-life of greater than 3, 4, 5, 6, or 7 days at a refrigerated temperature of 37° F.

The composition of this aspect, and including the substantially liquid/powdered mixture as described above, may be used in a non-liquid/powdered consumable food product. The non-liquid/powdered consumable food product is formed by heating of the composition of this aspect, e.g., by contacting the composition with a surface having a surface temperature of between 150° F. and 400° F. In some cases, the non-liquid/powdered consumable food product is an egg-less vegan scramble.

Any herein disclosed composition may be used as an ingredient in making an egg-less food product, e.g., an egg-less vegan scramble.

### Alternate Compositions

The present disclosure provides a composition comprising a mixture for preparation of, or replacement of, an egg-like product. The mixture comprising: (a) one or more recombinant egg-related proteins selected from group consisting of recombinant ovomucoid (rOVD), recombinant ovalbumin (rOVA), and recombinant lysozyme (rOVL), wherein one or more recombinant egg-related proteins are present in the mixture at a concentration of between 0.1% to 40% on a weight per weight basis; and (b) a plant-based protein component, wherein the plant-based protein component is present in the mixture at a concentration of between 0.1% and 30% on a weight per weight basis.

Any of the above-mentioned components (e.g., one or more egg-related proteins selected from the group consisting of a recombinant ovomucoid (rOVD), and a recombinant ovalbumin (rOVA), and a recombinant lysozyme (rOVL); (b) a protein component, wherein the protein component comprises a plant protein; (c) a dietary fiber-providing component, wherein the dietary fiber-providing component comprises a plant fiber; (d) a starch-providing component, wherein the starch-providing component comprises polysaccharides, e.g., having glucose monomers joined via α-1,4 linkages; (e) a gelation agent; (f) a salt and/or another flavoring agent; (g) a lipid component; and (h) water) and further components described above, may be included in the composition of this aspect. Moreover, the percentages for each of the above-mentioned components, described with respect to the substantially liquid mixture, may be consistent with the percentages in a composition of this aspect.

In embodiments of this aspect, the one or more recombinant egg-related proteins comprise rOVD. The rOVD is present in the mixture at a concentration of from about 0.1% to about 20% on a weight per weight or weight per volume. In various embodiments, the rOVD, is present in the mixture at a concentration from about 0.1% to about 20% on a weight per weigh (w/w) or weight per volume (w/v) basis. In embodiments, the rOVD is present in the substantially liquid mixture at a concentration of about 0.10%, 0.20%, 0.30%, 0.40%, 0.50%, 0.60%, 0.70%, 0.80%, 0.90%, or about 1.00% w/w or w/v. In some embodiments, the rOVD is present in the mixture at a concentration of about 1%, 2%, 3%, 4%, 5%, 6%, 7%, 8%, 9%, 10%, 11%, 12%, 13%, 14%, 15%, 16%, 17%, 18%, 19%, or about 20% w/w or w/v. In various embodiments, the rOVD is present in the mixture at a concentration of from about 1% to 3%, 2% to 5%, 4% to 7%, 6% to 9%, 8% to 11%, 10% to 13%, 12% to 15%, 14% to 17%, 16% to 19%, 18% to 20% w/w or w/v.

In embodiments of this aspect, the one or more recombinant egg-related proteins comprise rOVA, wherein the rOVA is present in the mixture at a concentration of from about 0.1% to about 40% w/w or w/v. In various embodiments, the rOVA, is present in the mixture at a concentration from about 0.1% to about 40% on a weight per weigh (w/w) or weight per volume (w/v) basis. In embodiments, the rOVA is present in the mixture at a concentration of about 0.10%, 0.20%, 0.30%, 0.40%, 0.50%, 0.60%, 0.70%, 0.80%, 0.90%, or about 1.00% w/w or w/v. In some embodiments, the rOVA is present in the mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v.

In various embodiments, the rOVA is present in the mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v.

In embodiments of this aspect, the one or more recombinant egg-related proteins comprise rOVL, wherein the rOVL is present in the mixture at a concentration of from about 0.1% to about 40% w/w or w/v. In various embodiments, the rOVL, is present in the mixture at a concentration from about 0.1% to about 40% on a weight per weigh (w/w) or weight per volume (w/v) basis. In embodiments, the rOVL is present in the mixture at a concentration of about 0.10%, 0.20%, 0.30%, 0.40%, 0.50%, 0.60%, 0.70%, 0.80%, 0.90%, or about 1.00% w/w or w/v. In some embodiments, the rOVL is present in the mixture at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% w/w or w/v. In various embodiments, the rOVL is present in the mixture at a concentration of from about 1% to 3%, 2% to 5%, 4% to 7%, 6% to 9%, 8% to 11%, 10% to 13%, 12% to 15%, 14% to 17%, 16% to 19%, 18% to 21%, 20% to 23%, 22% to 25%, 24% to 27%, 26% to 29%, 28% to 31%, 30% to 33%, 32% to 35%, 34% to 37%, 36% to 39%, or 38% to 40% w/w or w/v.

In embodiments of this aspect, the plant-based protein component is present in the mixture at a concentration from about 0.1%-30%, 0.5% to 25%, 2% to 20%, or 5% to 15% on a weight per weight basis.

In embodiments, the plant-based protein component is present in the mixture at a concentration of about 0.10%, 0.20%, 0.30%, 0.40%, 0.50%, 0.60%, 0.70%, 0.80%, 0.90%, or about 1.00% w/w or w/v. In some embodiments, the plant-based protein component is present in the mixture at a concentration of about 1%, 2%, 3%, 4%, 5%, 6%, 7%, 8%, 9%, 10%, 11%, 12%, 13%, 14%, 15%, 16%, 17%, 18%, 19%, or about 20% w/w or w/v. In various embodiments, the plant-based protein component is present in the mixture at a concentration of from about 1% to 3%, 2% to 5%, 4% to 7%, 6% to 9%, 8% to 11%, 10% to 13%, 12% to 15%, 14% to 17%, 16% to 19%, or 18% to 20% w/w or w/v.

In some embodiments of this aspect, the composition and/or the mixture is substantially devoid of cholesterol.

In some various embodiments of this aspect, the composition exhibits, upon cooking, a gelling capability that is equal to or exceeds the gelling capability of one or both of natural egg white or a whole egg.

In embodiments of this aspect, the composition has a shelf-life of greater than 3, 4, 5, 6, or 7 days at a refrigerated temperature of 37° F.

The composition of this aspect, and including the mixture as described above, may be used in a non-liquid consumable food product. The non-liquid consumable food product is formed by heating of the composition of this aspect, e.g., by contacting the composition with a surface having a surface temperature of between 150° F. and 400° F. In some cases, the non-liquid consumable food product is an egg-less vegan scramble.

Any herein disclosed composition may be used as an ingredient in making an egg-less food product, e.g., an egg-less vegan scramble.

### Formation of Non-Liquid Consumable Food Product

In some embodiments, heating of the composition may cause the formation of a non-liquid consumable food product. In some embodiments, a substantially liquid mixture or a mixture is added to other ingredients and then heated. In some embodiments, the substantially liquid mixture or the mixture is heated alone. In some embodiments, the non-liquid consumable food product is formed when a composition is contacted with a surface having a temperature of about 150° F., about 200° F., about 250° F., about 300° F., about 350° F., or about 400° F. In some embodiments, the non-liquid consumable food product is formed when heated to a temperature of 150° F. to 400° F. In some embodiments, the non-liquid consumable food product is formed when heated to a temperature of 150° F. to 200° F., 150° F. to 250° F., 150° F. to 300° F., 150° F. to 350° F., 150° F. to 400° F., 200° F. to 250° F., 200° F. to 300° F., 200° F. to 350° F., 200° F. to 400° F., 250° F. to 300° F., 250° F. to 350° F., 250° F. to 400° F., 300° F. to 350° F., 300° F. to 400° F., or 350° F. to 400° F. In some embodiments, the non-liquid consumable food product is formed when heated to a temperature of 150° F., 200° F., 250° F., 300° F., 350° F., or 400° F. In some embodiments, the non-liquid consumable food product is formed when heated to a temperature of at least 150° F., 200° F., 250° F., 300° F., or 350° F. In some embodiments, the non-liquid consumable food product is formed when heated to a temperature of at most 200° F., 250° F., 300° F., 350° F., or 400° F.

In some embodiments, the non-liquid consumable food product is suitable for consumption (i.e., human consumption and/or animal consumption). In some embodiments, the non-liquid consumable food product is formed by heating comprises an egg-less food product. In some embodiments, the non-liquid consumable food product is vegan. In some embodiments, the egg-less food product comprises an egg-less vegan scramble.

### Methods of Using the Composition.

Also disclosed herein, in certain embodiments, are methods of using the composition described herein. Natural eggs have a wide range of uses, such as in baking, cooking, and in making beverages. The compositions disclosed herein may have multiple uses, such as being used as a replacement for eggs in a wide range of food products. In some embodiments, a food product suitable for consumption (i.e., human consumption and/or animal consumption) is formed when the composition is heated. The composition described herein may be used as an egg-substitute in such uses. In some embodiments, the composition is used alone as a food-product, such that the composition may be consumed without additional ingredients. In some embodiments, the composition is used as an ingredient in making an egg-less food product. In some embodiments, the egg-less food product is an egg-less vegan scramble. In some embodiments, the egg-less food product is a baked food item, beverage, or cooked food item.

### Methods for Preparing a Substantially Liquid/Powdered Mixture.

In some embodiments, preparation of the substantially liquid/powdered mixture or alternate mixture comprises combining any one or more of the aforementioned components described herein (i.e., one or more recombinant proteins, one or more plant proteins, a dietary fiber-providing component, a starch-providing component, a gelation agent, a salt and/or another flavoring agent, a lipid component and water, optionally with any further component described herein) in a container to form a liquid/powdered mixture. In some embodiments, preparation of the liquid mixture comprises combining one or more recombinant proteins, one or more plant proteins, a dietary fiber-providing component, a starch-providing component, a gelation agent, a salt and/or another flavoring agent, a lipid component and water in a container to for a liquid mixture. In some embodiments, the liquid mixture comprises a solution, suspension or colloid. In some embodiments, the mixture is homogenous.

In some embodiments, the composition may be used as a food product, where the composition can be used as a food or an ingredient in a food composition. The liquid/powdered mixture may be used alone, or as an ingredient in a food composition.

### Uses and Advantages of Compositions of the Present Disclosure

The composition described herein may be used as a substitute for whole egg, egg white, or as a comparable egg replacement composition. In some embodiments, the composition, when used alone or in combination with other food ingredients, will provide a nutritional feature, such as protein content, protein fortification, and amino acid content. In some embodiments, the composition will exhibit equivalent characteristics to that of a natural egg white, a whole egg, or a comparable composition. In some embodiments, the nutritional value of the composition will be comparable, substantially similar to, or better than that of a natural egg, egg white or comparable composition. In some embodiments, the composition will exhibit one or more functional features when used alone, or in combination with other food products. In some embodiments, the composition will exhibit a hardness, adhesiveness, fracturability, cohesiveness, gumminess, gelatinous texture, chewiness, or a combination of such characteristics that is at least equivalent to that of a natural egg white, a whole egg, or a comparable composition without the fiber-providing component. In some embodiments, the composition, when cooked, will exhibit a hardness, adhesiveness, fracturability, cohesiveness, gumminess, gelatinous texture, or chewiness that is at least equivalent to that of a natural egg white, a whole egg, or a comparable composition without the fiber-providing component when cooked. In some embodiments, the composition, when uncooked, will exhibit a hardness, adhesiveness, fracturability, cohesiveness, gumminess, gelatinous texture, or chewiness that is at least equivalent to that of a natural egg white, a whole egg, or a comparable composition without the fiber-providing component when uncooked.

In some embodiments, the composition provides sensory neutrality, or improved sensory appeal or similar sensory appeal as to a whole egg, egg white, or a comparable egg replacement composition. As used herein “sensory neutrality” refers to absence of a strong or distinctive taste, odor (smell) or combination thereof, as well as texture, hardness, adhesiveness, fracturability, cohesiveness, gumminess, gelatinous texture, chewiness, and overall appearance. A panel of trained analysts, such as the one described in Kemp et al. 2009 may be used for the detection of sensory information. Sensory neutrality may be beneficial to some consumers, as the composition may be used to provide a different characteristic, such as an improved protein content.

In some embodiments, the composition will exhibit a hardness that is less than, equal to, or more than that of an egg white, a whole egg, or a comparable composition without the fiber-providing component. Hardness refers to the resistance to localized plastic deformation induced by either mechanical indentation or abrasion. In some embodiments, the composition will exhibit a hardness that is at least equivalent to that of an egg white, a whole egg, or a comparable composition without the fiber-providing component.

In some embodiments, the composition will exhibit an adhesiveness that is less than, equal to, or more than that of an egg white, a whole egg, or a comparable composition without the fiber-providing component. Adhesiveness refers to the property of sticking together or the joining of surfaces of different composition. In some embodiments, the composition will exhibit an adhesiveness that is at least equivalent to that of an egg white, a whole egg, or a comparable composition without the fiber-providing component.

In some embodiments, the composition will exhibit a fracturability that is less than, equal to, or more than that of an egg white, a whole egg, or a comparable composition without the fiber-providing component. Fracturability refers to the property of being capable of fracture or breaking. The term is similar to that of being brittle. In some embodiments, the composition will exhibit a fracturability that is at least equivalent to that of an egg white, a whole egg, or a comparable composition without the fiber-providing component.

In some embodiments, the composition will exhibit a cohesiveness that is less than, equal to, or more than that of an egg white, a whole egg, or a comparable composition without the fiber-providing component. Cohesiveness refers to the property of being attracted to other molecules of the same kind. In some embodiments, the composition will exhibit a cohesiveness that is at least equivalent to that of an egg white, a whole egg, or a comparable composition without the fiber-providing component.

In some embodiments, the composition will exhibit a gumminess that is less than, equal to, or more than that of an egg white, a whole egg, or a comparable composition without the fiber-providing component. The term “gumminess” refers to the property of being sticky and viscous. In some embodiments, the composition will exhibit a gumminess that is at least equivalent to that of an egg white, a whole egg, or a comparable composition without the fiber-providing component.

In some embodiments, the composition will exhibit a gelatinous texture that is less than, equal to, or more than that of an egg white, a whole egg, or a comparable composition without the fiber-providing component. The term “gelatinous texture” refers to the property of having a texture that resembles a gelatin or jelly. In some embodiments, the composition will exhibit a gelatinous texture that is at least equivalent to that of an egg white, a whole egg, or a comparable composition without the fiber-providing component.

In some embodiments, the composition will exhibit a chewiness that is less than, equal to, or more than that of an egg white, a whole egg, or a comparable composition without the fiber-providing component. The term “chewiness” refers to the property of mouthfeel sensation of labored mastication due to sustained, elastic resistance from a food, or to the energy required to chew a solid food until it is ready for swallowing. Chewiness may be measured by the relationship of hardness×cohesiveness x elasticity. In some embodiments, the composition will exhibit a chewiness that is at least equivalent to that of an egg white, a whole egg, or a comparable composition without the fiber-providing component.

In some embodiments, the composition exhibits a gelling capability that is equal to or exceeds the gelling capability of one or both of natural egg white or a whole egg. The liquid/powdered mixture may be formulated to exhibit characteristics similar to those of a natural egg white, natural whole egg, or a comparable composition. Gelling characteristics of the liquid/powdered mixture may be provided by gelling agent, such as recombinant goose lysozyme. In some embodiments, the composition, upon cooking, exhibits a gelling capability that is equal to or exceeds the gelling capability of one or both of natural egg white or a whole egg.

In some embodiments, the composition exhibits hardness, adhesiveness, fracturability, cohesiveness, gumminess, gelatinous texture, and/or chewiness that is at least equivalent to that of a natural egg white or a whole egg.

In some embodiments, the composition exhibits hardness, adhesiveness, fracturability, cohesiveness, gumminess, gelatinous texture, and/or chewiness that is at least equivalent to that of a natural egg white, a whole egg, or a comparable composition without the dietary fiber-providing component.

In some embodiments, the composition comprises similar taste characteristics to a natural egg, egg white or comparable composition when cooked. Taste, or lack thereof, is an important aspect of the composition. In some embodiments, the composition has taste characteristics that are similar to a natural whole egg, egg white or comparable composition. In some embodiments, the composition comprises a salty taste, a savory taste, a sweet taste, a bitter taste, an umami taste, or any combination thereof.

In some embodiments, the composition has an odor similar to a natural whole egg, egg white or comparable composition. In some embodiments, the composition, when cooked, has an odor similar to a natural whole egg, egg white or comparable composition. In some embodiments, the composition, when cooked, has less odor than a natural whole egg, egg white or comparable composition. In some embodiments, the composition does not have an odor.

In some embodiments, the composition provides for a reduction in odor and/or taste. In some embodiments, the composition has less of an “egg-like” odor or taste as compared to a natural egg, egg white or comparable composition. In some embodiments the composition does not have a taste or odor.

In some embodiments, the composition provides a texture to a food product, similar or substantially similar to the texture provided by a natural egg, egg white or comparable composition. The compositions disclosed herein can be a liquid/powdered, semi-solid or solid. In some embodiments, the composition comprises a texture that is the same or substantially similar to that of a natural egg, egg white or comparable composition.

In some embodiments, the composition comprises similar functional features to a natural egg, egg white, or a similar egg replacement composition. The composition comprises or can provide one or more characteristics, such as foaming, gelling, whipping, fluffing, binding, springiness, aeration, or creaminess. In some embodiments, the characteristics of the composition are the same or better than that of a natural egg, egg white or comparable composition.

In some embodiments, the composition comprises a foaming capability, foaming capacity, foam height, and/or foaming stability similar to or better than a natural egg, egg white or comparable composition. For example, the composition may be used for forming a foam for use in baked products, such as cakes, meringues and other foods.

In some embodiments, the composition provides structure, texture or a combination of structure and texture. In some embodiments, the composition provides the structure and texture of that provided by a natural egg, egg white or comparable composition. In some embodiments, the composition can be used in place of natural egg, egg white or a comparable composition in baked, boiled, poached, steamed, braised, roasted, grilled, fried, sautéed, blanched, or microwaved goods. In some embodiments, the composition is added to a food ingredient or food product the composition provides structure, texture or a combination of structure and texture to the baked product.

In some embodiments, the composition, comprises an overall appearance that is the same or substantially similar to a natural egg, egg white or comparable composition that is cooked and/or uncooked. In some embodiments, the composition, comprises a color that is the same or substantially similar to a natural egg, egg white or comparable composition that is cooked and/or uncooked. In some embodiments, the composition does not comprise a color that is the same or substantially similar to a natural egg, egg white or comparable composition that is cooked and/or uncooked.

In some embodiments, the composition will have a desirable shelf-life. In Preferably, the composition will be capable of being stored for a period of time. some embodiments, the shelf-life is greater than about 3 days, about 4 days, about 5 days, about 6 days, about 7 days, about 8 days, about 9 days, or about 10 days. In some embodiments, the shelf-life exists as the period stated herein when kept cool (i.e., refrigerated and/or kept at a temperature of about 37° F. or below).

Since various compositions lack any animal products, these may lack components that are harmful to human health. For example, some compositions will lack saturated fat. In some embodiments, the composition is substantially devoid of cholesterol. In some embodiments, the composition is substantially devoid of animal-based cholesterol. Thus, the composition may be useful to those with certain conditions (i.e., cardiovascular disease) due to the ability to the composition's lack of animal or animal egg derived components.

Exemplary OVD, OVA and OVL amino acid sequences contemplated herein are provided in Table 1 below as SEQ ID NOs: 1-44, 45-118, 119-129, respectively.

### Recombinant Protein Production

In any composition described herein, the protein may be recombinantly expressed in a host cell. The recombinant protein may be OVD, OVA, OVL, or other recombinant proteins.

rOVD, rOVA or rOVL can have an amino acid sequence from any species. For example, an rOVD, rOVA and/or rOVL can have an amino acid sequence of OVD native to a bird (avian) or a reptile or platypus. A recombinant protein such as rOVD, rOVA and/or rOVL having an amino acid sequence from an avian OVD and/or OVA can be selected from the group consisting of: poultry, fowl, waterfowl, game bird, chicken, quail, turkey, turkey vulture, hummingbird, duck, ostrich, goose, gull, guineafowl, pheasant, emu, and any combination thereof. A recombinant protein such as rOVD, rOVA and/or rOVL can have an amino acid sequence native to a single species, such as Gallus gallus domesticus. Alternatively, a recombinant protein such as rOVD, rOVA and/or rOVL can have an amino acid sequence native to two or more species, and as such be a hybrid.

Exemplary OVD, OVA and/or rOVL amino acid sequences contemplated herein are provided in Table 1 below as SEQ ID NOs: 1-44, 45-118, and 119-129, respectively.

TABLE 1
Sequences

| | SEQ ID | |
|---|---|---|
| Sequence Description | NOs | SEQUENCES |
| Ovomucoid (canonical) | SEQ ID | AEVDCSRFPNATDKEGKDVLVCNKDLRPICGTDGVTYTNDCLLCAYSIEFGTNISKEHDGECKETV |
| mature chicken OVD | NO: 1 | PMNCSSYANTTSEDGKVMVLCNRAFNPVCGTDGVTYDNECLLCAHKVEQGASVDKRHDGGCRK |
| | | ELAAVSVDCSEYPKPDCTAEDRPLCGSDNKTYGNKCNFCNAVVESNGTLTLSHFGKC |
| Ovomucoid | SEQ ID | AEVDCSRFPNATDMEGKDVLVCNKDLRPICGTDGVTYTNDCLLCAYSVEFGTNISKEHDGECKET |
| variant of SEQ ID 1 | NO: 2 | VPMNCSSYANTTSEDGKVMVLCNRAFNPVCGTDGVTYDNECLLCAHKVEQGASVDKRHDGGCR |
| | | KELAAVSVDCSEYPKPDCTAEDRPLCGSDNKTYGNKCNFCNAVVESNGTLTLSHFGKC |
| G162M F167A | SEQ ID | AEVDCSRFPNATDMEGKDVLVCNKDLRPICGTDGVTYTNDCLLCAYSVEFGTNISKEHDGECKET |
| Ovomucoid | NO: 3 | VPMNCSSYANTTSEDGKVMVLCNRAFNPVCGTDGVTYDNECLLCAHKVEQGASVDKRHDGGCR |
| Variant of Chicken | | KELAAVSVDCSEYPKPDCTAEDRPLCGSDNKTYMNKCNACNAVVESNGTLTLSHFGKC |
| OVD in Genbank | | |
| Ovomucoid isoform 1 | SEQ ID | MAMAGVFVLFSFVLCGFLPDAAFGAEVDCSRFPNATDKEGKDVLVCNKDLRPICGTDGVTYTND |
| precursor full length | NO: 4 | CLLCAYSIEFGTNISKEHDGECKETVPMNCSSYANTTSEDGKVMVLCNRAFNPVCGTDGVTYDNE |
| | | CLLCAHKVEQGASVDKRHDGGCRKELAAVSVDCSEYPKPDCTAEDRPLCGSDNKTYGNKCNFCN |
| | | AVVESNGTLTLSHFGKC |
| Ovomucoid [Gallus | SEQ ID | MAMAGVFVLFSFVLCGFLPDAVFGAEVDCSRFPNATDMEGKDVLVCNKDLRPICGTDGVTYTND |
| gallus] | NO: 5 | CLLCAYSVEFGTNISKEHDGECKETVPMNCSSYANTTSEDGKVMVLCNRAFNPVCGTDGVTYDNE |
| | | CLLCAHKVEQGASVDKRHDGGCRKELAAVSVDCSEYPKPDCTAEDRPLCGSDNKTYGNKCNFCN |
| | | AVVESNGTLTLSHFGKC |
| Ovomucoid isoform 2 | SEQ ID | MAMAGVFVLFSFVLCGFLPDAAFGAEVDCSRFPNATDKEGKDVLVCNKDLRPICGTDGVTYTND |
| precursor [Gallus | NO: 6 | CLLCAYSIEFGTNISKEHDGECKETVPMNCSSYANTTSEDGKVMVLCNRAFNPVCGTDGVTYDNE |
| gallus] | | CLLCAHKVEQGASVDKRHDGGCRKELAAVDCSEYPKPDCTAEDRPLCGSDNKTYGNKCNFCNAV |
| | | VESNGTLTLSHFGKC |
| Ovomucoid [Gallus | SEQ ID | AEVDCSRFPNATDKEGKDVLVCNKDLRPICGTDGVTYNNECLLCAYSIEFGTNISKEHDGECKETV |
| gallus] | NO: 7 | PMNCSSYANTTSEDGKVMVLCNRAFNPVCGTDGVTYDNECLLCAHKVEQGASVDKRHDGECRK |
| | | ELAAVSVDCSEYPKPDCTAEDRPLCGSDNKTYGNKCNFCNAVVESNGTLTLSHFGKC |
| Ovomucoid [Numida | SEQ ID | MAMAGVFVLFSFALCGFLPDAAFGVEVDCSRFPNATNEEGKDVLVCTEDLRPICGTDGVTYSNDC |
| meleagris] | NO: 8 | LLCAYNIEYGTNISKEHDGECREAVPVDCSRYPNMTSEEGKVLILCNKAFNPVCGTDGVTYDNECL |
| | | LCAHNVEQGTSVGKKHDGECRKELAAVDCSEYPKPACTMEYRPLCGSDNKTYDNKCNFCNAVV |
| | | ESNGTLTLSHFGKC |
| PREDICTED: | SEQ ID | MQTITWRQPQGDHLRSRAPAATCRAGQYLTMAMAGIFVLFSFALCGFLPDAAFGVEVDCSRFPNT |
| Ovomucoid isoform X1 | NO: 9 | TNEEGKDVLVCTEDLRPICGTDGVTHSECLLCAYNIEYGTNISKEHDGECREAVPMDCSRYPNTTN |
| [Meleagris gallopavo] | | EEGKVMILCNKALNPVCGTDGVTYDNECVLCAHNLEQGTSVGKKHDGGCRKELAAVSVDCSEYP |
| | | KPACTLEYRPLCGSDNKTYGNKCNFCNAVVESNGTLTLSHFGKC |
| Ovomucoid [Meleagris | SEQ ID | VEVDCSRFPNTTNEEGKDVLVCTEDLRPICGTDGVTHSECLLCAYNIEYGTNISKEHDGECREAVP |
| gallopavo] | NO: 10 | MDCSRYPNTTSEEGKVMILCNKALNPVCGTDGVTYDNECVLCAHNLEQGTSVGKKHDGECRKEL |
| | | AAVSVDCSEYPKPACTLEYRPLCGSDNKTYGNKCNFCNAVVESNGTLTLSHFGKC |
| PREDICTED: | SEQ ID | MQTITWRQPQGDHLRSRAPAATCRAGQYLTMAMAGIFVLFSFALCGFLPDAAFGVEVDCSRFPNT |
| Ovomucoid isoform X2 | NO: 11 | TNEEGKDVLVCTEDLRPICGTDGVTHSECLLCAYNIEYGTNISKEHDGECREAVPMDCSRYPNTTN |
| [Meleagris gallopavo] | | EEGKVMILCNKALNPVCGTDGVTYDNECVLCAHNLEQGTSVGKKHDGGCRKELAAVDCSEYPKP |
| | | ACTLEYRPLCGSDNKTYGNKCNFCNAVVESNGTLTLSHFGKC |
| Ovomucoid | SEQ ID | EYGTNISIKHNGECKETVPMDCSRYANMTNEEGKVMMPCDRTYNPVCGTDGVTYDNECQLCAH |
| [Bambusicola | NO: 12 | NVEQGTSVDKKHDGVCGKELAAVSVDCSEYPKPECTAEERPICGSDNKTYGNKCNFCNAVVYVQ |
| thoracicus] | | P |
| Ovomucoid [Callipepla | SEQ ID | VDCSRFPNTTNEEGKDVLACTKELHPICGTDGVTYSNECLLCYYNIEYGTNISKEHDGECTEAVPV |
| squamata] | NO: 13 | DCSRYPNTTSEEGKVLIPCNRDFNPVCGSDGVTYENECLLCAHNVEQGTSVGKKHDGGCRKEFAA |
| | | VSVDCSEYPKPDCTLEYRPLCGSDNKTYASKCNFCNAVVIWEQEKNTRHHASHSVFFISARLVC |
| Ovomucoid [Colinus | SEQ ID | MLPLGLREYGTNTSKEHDGECTEAVPVDCSRYPNTTSEEGKVRILCKKDINPVCGTDGVTYDNECL |
| virginianus] | NO: 14 | LCSHSVGQGASIDKKHDGGCRKEFAAVSVDCSEYPKPACMSEYRPLCGSDNKTYVNKCNFCNAV |
| | | VYVQPWLHSRCRLPPTGTSFLGSEGRETSLLTSRATDLQVAGCTAISAMEATRAAALLGLVLLSSF |
| | | CELSHLCFSQASCDVYRLSGSRNLACPRIFQPVCGTDNVTYPNECSLCRQMLRSRAVYKKHDGRC |
| | | VKVDCTGYMRATGGLGTACSQQYSPLYATNGVIYSNKCTFCSAVANGEDIDLLAVKYPEEESWIS |
| | | VSPTPWRMLSAGA |
| Ovomucoid-like | SEQ ID | MSWWGIKPALERPSQEQSTSGQPVDSGSTSTTTMAGIFVLLSLVLCCFPDAAFGVEVDCSRFPNTT |
| isoform X2 [Anser | NO: 15 | NEEGKEVLLCTKDLSPICGTDGVTYSNECLLCAYNIEYGTNISKDHDGECKEAVPVDCSTYPNMTN |
| cygnoides domesticus] | | EEGKVMLVCNKMFSPVCGTDGVTYDNECMLCAHNVEQGTSVGKKYDGKCKKEVATVDCSDYP |
| | | KPACTVEYMPLCGSDNKTYDNKCNFCNAVVDSNGTLTLSHFGKC |
| Ovomucoid-like | SEQ ID | MSSQNQLHRRRRPLPGGQDLNKYYWPHCTSDRFSWLLHVTAEQFRHCVCIYLQPALERPSQEQST |
| isoform X1 [Anser | NO: 16 | SGQPVDSGSTSTTTMAGIFVLLSLVLCCFPDAAFGVEVDCSRFPNTTNEEGKEVLLCTKDLSPICGT |
| cygnoides domesticus] | | DGVTYSNECLLCAYNIEYGTNISKDHDGECKEAVPVDCSTYPNMTNEEGKVMLVCNKMFSPVCG |
| | | TDGVTYDNECMLCAHNVEQGTSVGKKYDGKCKKEVATVDCSDYPKPACTVEYMPLCGSDNKTY |
| | | DNKCNFCNAVVDSNGTLTLSHFGKC |
| Ovomucoid [Coturnix | SEQ ID | VEVDCSRFPNTTNEEGKDEVVCPDELRLICGTDGVTYNHECMLCFYNKEYGTNISKEQDGECGET |
| japonica] | NO: 17 | VPMDCSRYPNTTSEDGKVTILCTKDFSFVCGTDGVTYDNECMLCAHNVVQGTSVGKKHDGECRK |
| | | ELAAVSVDCSEYPKPACPKDYRPVCGSDNKTYSNKCNFCNAVVESNGTLTLNHFGKC |
| Ovomucoid [Coturnix | SEQ ID | MAMAGVFLLFSFALCGFLPDAAFGVEVDCSRFPNTTNEEGKDEVVCPDELRLICGTDGVTYNHEC |
| japonica] | NO: 18 | MLCFYNKEYGTNISKEQDGECGETVPMDCSRYPNTTSEDGKVTILCTKDFSFVCGTDGVTYDNEC |
| | | MLCAHNIVQGTSVGKKHDGECRKELAAVSVDCSEYPKPACPKDYRPVCGSDNKTYSNKCNFCNA |
| | | VVESNGTLTLNHFGKC |
| Ovomucoid [Anas | SEQ ID | MAGVFVLLSLVLCCFPDAAFGVEVDCSRFPNTTNEEGKDVLLCTKELSPVCGTDGVTYSNECLLC |
| platyrhynchos] | NO: 19 | AYNIEYGTNISKDHDGECKEAVPADCSMYPNMTNEEGKMTLLCNKMFSPVCGTDGVTYDNECML |
| | | CAHNVEQGTSVGKKYDGKCKKEVATVDCSGYPKPACTMEYMPLCGSDNKTYGNKCNFCNAVV |
| | | DSNGTLTLSHFGEC |
| Ovomucoid, partial | SEQ ID | QVDCSRFPNTTNEEGKEVLLCTKELSPVCGTDGVTYSNECLLCAYNIEYGTNISKDHDGECKEAVP |
| [Anas platyrhynchos] | NO: 20 | ADCSMYPNMTNEEGKMTLLCNKMFSPVCGTDGVTYDNECMLCAHNVEQGTSVGKKYDGKCKK |
| | | EVATVSVDCSGYPKPACTMEYMPLCGSDNKTYGNKCNFCNAVV |
| Ovomucoid-like [Tyto | SEQ ID | MTMPGAFVVLSFVLCCFPDATFGVEVDCSTYPNTTNEEGKEVLVCSKILSPICGTDGVTYSNECLL |
| alba] | NO: 21 | CANNIEYGTNISKYHDGECKEFVPVNCSRYPNTTNEEGKVMLICNKDLSPVCGTDGVTYDNECLL |
| | | CAHNLEPGTSVGKKYDGECKKEIATVDCSDYPKPVCSLESMPLCGSDNKTYSNKCNFCNAVVDSN |
| | | ETLTLSHFGKC |
| Ovomucoid [Balearica | SEQ ID | MTMAGVFVLLSFALCCFPDAAFGVEVDCSTYPNTTNEEGKEVLVCTKILSPICGTDGVTYSNECLL |
| regulorum gibbericeps] | NO: 22 | CAYNIEYGTNVSKDHDGECKEVVPVDCSRYPNSTNEEGKVVMLCSKDLNPVCGTDGVTYDNECV |
| | | LCAHNVESGTSVGKKYDGECKKETATVDCSDYPKPACTLEYMPFCGSDSKTYSNKCNFCNAVVD |
| | | SNGTLTLSHFGKC |
| Turkeyvulture | SEQ ID | MTTAGVFVLLSFALCSFPDAAFGVEVDCSTYPNTTNEEGKEVLVCTKILSPICGTDGVTYSNECLL |
| [Cathartes aura] OVD | NO: 23 | CAYNIEYGTNVSKDHDGECKEFVPVDCSRYPNTTNEDGKVVLLCNKDLSPICGTDGVTYDNECLL |
| (native sequence) | | CARNLEPGTSVGKKYDGECKKEIATVDCSDYPKPVCSLEYMPLCGSDSKTYSNKCNFCNAVVDSN |
| bolded is native signal | | GTLTLSHFGKC |
| sequence | | |
| Ovomucoid-like | SEQ ID | MTTAGVFVLLSFTLCSFPDAAFGVEVDCSPYPNTTNEEGKEVLVCNKILSPICGTDGVTYSNECLLC |
| [Cuculus canorus] | NO: 24 | AYNLEYGTNISKDYDGECKEVAPVDCSRHPNTTNEEGKVELLCNKDLNPICGTNGVTYDNECLLC |
| | | ARNLESGTSIGKKYDGECKKEIATVDCSDYPKPVCTLEEMPLCGSDNKTYGNKCNFCNAVVDSNG |
| | | TLTLSHFGKC |
| Ovomucoid | SEQ ID | MTTAVVFVLLSFALCCFPDAAFGVEVDCSTYPNSTNEEGKDVLVCPKILGPICGTDGVTYSNECLL |
| [Antrostomus | NO: 25 | CAYNIQYGTNVSKDHDGECKEIVPVDCSRYPNTTNEEGKVVFLCNKNFDPVCGTDGDTYDNECM |
| carolinensis] | | LCARSLEPGTTVGKKHDGECKREIATVDCSDYPKPTCSAEDMPLCGSDSKTYSNKCNFCNAVVDS |
| | | NGTLTLSRFGKC |
| Ovomucoid [Cariama | SEQ ID | MTMTGVFVLLSFAICCFPDAAFGVEVDCSTYPNTTNEEGKEVLVCTKILSPICGTDGVTYSNECLLC |
| cristata] | NO: 26 | AYNIEYGTNVSKDHDGECKEVVPVDCSKYPNTTNEEGKVVLLCSKDLSPVCGTDGVTYDNECLLC |
| | | ARNLEPGSSVGKKYDGECKKEIATIDCSDYPKPVCSLEYMPLCGSDSKTYDNKCNFCNAVVDSNG |
| | | TLTLSHFGKC |
| Ovomucoid-like | SEQ ID | MTTAGVFVLLSFVLCCFPDAVFGVEVDCSTYPNTTNEEGKEVLVCTKILSPICGTDGVTYSNECLLC |
| isoform X2 [Pygoscelis | NO: 27 | AYNIEYGTNVSKDHDGECKEVVPVNCSRYPNTTNEEGKVVLRCSKDLSPVCGTDGVTYDNECLM |
| adeliae] | | CARNLEPGAVVGKNYDGECKKEIATVDCSDYPKPVCSLEYMPLCGSDSKTYSNKCNFCNAVVDS |
| | | NGTLTLSHFGKC |
| Ovomucoid-like | SEQ ID | MTTAGVFVLLSIALCCFPDAAFGVEVDCSAYSNTTSEEGKEVLSCTKILSPICGTDGVTYSNECLLC |
| [Nipponia nippon] | NO: 28 | AYNIEYGTNISKDHDGECKEVVSVDCSRYPNTTNEEGKAVLLCNKDLSPVCGTDGVTYDNECLLC |
| | | AHNLEPGTSVGKKYDGACKKEIATVDCSDYPKPVCTLEYLPLCGSDSKTYSNKCDFCNAVVDSNG |
| | | TLTLSHFGKC |
| Ovomucoid-like | SEQ ID | MTTAGVFVLLSFALCCFPDAAFGVEVDCSTYPNTTNEEGKEVLVCTKILSPICGTDGTTYSNECLLC |
| [Phaethon lepturus] | NO: 29 | AYNIEYGTNVSKDHDGECKVVPVDCSKYPNTTNEDGKVVLLCNKALSPICGTDRVTYDNECLMC |
| | | AHNLEPGTSVGKKHDGECQKEVATVDCSDYPKPVCSLEYMPLCGSDGKTYSNKCNFCNAVVNSN |
| | | GTLTLSHFEKC |
| Ovomucoid-like | SEQ ID | MTTAGVFVLLSFVLCCFFPDAAFGVEVDCSTYPNTTNEEGKEVLVCAKILSPVCGTDGVTYSNECL |
| isoform X1 | NO: 30 | LCAHNIENGTNVGKDHDGKCKEAVPVDCSRYPNTTDEEGKVVLLCNKDVSPVCGTDGVTYDNEC |
| [Melopsittacus | | LLCAHNLEAGTSVDKKNDSECKTEDTTLAAVSVDCSDYPKPVCTLEYLPLCGSDNKTYSNKCRFC |
| undulatus] | | NAVVDSNGTLTLSRFGKC |
| Ovomucoid [Podiceps | SEQ ID | MTTAGVFVLLSFALCCSPDAAFGVEVDCSTYPNTTNEEGKEVLACTKILSPICGTDGVTYSNECLLC |
| cristatus] | NO: 31 | AYNMEYGTNVSKDHDGKCKEVVPVDCSRYPNTTNEEGKVVLLCNKDLSPVCGTDGVTYDNECL |
| | | LCARNLEPGASVGKKYDGECKKEIATVDCSDYPKPVCSLEHMPLCGSDSKTYSNKCTFCNAVVDS |
| | | NGTLTLSHFGKC |
| Ovomucoid-like | SEQ ID | MTTAGVFVLLSFALCCFPDAAFGVEVDCSTYPNTTNEEGREVLVCTKILSPICGTDGVTYSNECLLC |
| [Fulmarus glacialis] | NO: 32 | AYNIEYGTNVSKDHDGECKEVAPVGCSRYPNTTNEEGKVVLLCNKDLSPVCGTDGVTYDNECLL |
| | | CARHLEPGTSVGKKYDGECKKEIATVDCSDYPKPVCSLEYMPLCGSDSKTYSNKCNFCNAVLDSN |
| | | GTLTLSHFGKC |
| Ovomucoid | SEQ ID | MTTAGVFVLLSFALCCFPDAVFGVEVDCSTYPNTTNEEGKEVLVCTKILSPICGTDGVTYSNECLLC |
| [Aptenodytes forsteri] | NO: 33 | AYNIEYGTNVSKDHDGECKEVVPVDCSRYPNTTNEEGKVVLRCNKDLSPVCGTDGVTYDNECLM |
| | | CARNLEPGAIVGKKYDGECKKEIATVDCSDYPKPVCSLEYMPLCGSDSKTYSNKCNFCNAVVDSN |
| | | GTLILSHFGKC |
| Ovomucoid-like | SEQ ID | MTTAGVFVLLSFVLCCFPDAVFGVEVDCSTYPNTTNEEGKEVLVCTKILSPICGTDGVTYSNECLLC |
| isoform X1 [Pygoscelis | NO: 34 | AYNIEYGTNVSKDHDGECKEVVPVDCSRYPNTTNEEGKVVLRCSKDLSPVCGTDGVTYDNECLM |
| adeliae] | | CARNLEPGAVVGKNYDGECKKEIATVDCSDYPKPVCSLEYMPLCGSDSKTYSNKCNFCNAVVDS |
| | | NGTLTLSHFGKC |
| Ovomucoid isoform X1 | SEQ ID | MSSQNQLPSRCRPLPGSQDLNKYYQPHCTGDRFCWLFYVTVEQFRHCICIYLQLALERPSHEQSGQ |
| [Aptenodytes forsteri] | NO: 35 | PADSRNTSTMTTAGVFVLLSFALCCFPDAVFGVEVDCSTYPNTTNEEGKEVLVCTKILSPICGTDGV |
| | | TYSNECLLCAYNIEYGTNVSKDHDGECKEVVPVDCSRYPNTTNEEGKVVLRCNKDLSPVCGTDGV |
| | | TYDNECLMCARNLEPGAIVGKKYDGECKKEIATVDCSDYPKPVCSLEYMPLCGSDSKTYSNKCNF |
| | | CNAVVDSNGTLILSHFGKC |
| Ovomucoid, partial | SEQ ID | MTTAVVFVLLSFALCCFPDAAFGVEVDCSTYPNSTNEEGKDVLVCPKILGPICGTDGVTYSNECLL |
| [Antrostomus | NO: 36 | CAYNIQYGTNVSKDHDGECKEIVPVDCSRYPNTTNEEGKVVFLCNKNFDPVCGTDGDTYDNECM |
| carolinensis] | | LCARSLEPGTTVGKKHDGECKREIATVDCSDYPKPTCSAEDMPLCGSDSKTYSNKCNFCNAVV |
| rOVD as expressed in | SEQ ID | EAEAAEVDCSRFPNATDKEGKDVLVCNKDLRPICGTDGVTYTNDCLLCAYSIEFGTNISKEHDGEC |
| pichia secreted form 1 | NO: 37 | KETVPMNCSSYANTTSEDGKVMVLCNRAFNPVCGTDGVTYDNECLLCAHKVEQGASVDKRHDG |
| | | GCRKELAAVSVDCSEYPKPDCTAEDRPLCGSDNKTYGNKCNFCNAVVESNGTLTLSHFGKC |
| rOVD as expressed in | SEQ ID | EEGVSLEKREAEAAEVDCSRFPNATDKEGKDVLVCNKDLRPICGTDGVTYTNDCLLCAYSIEFGTN |
| pichia secreted form 2 | NO: 38 | ISKEHDGECKETVPMNCSSYANTTSEDGKVMVLCNRAFNPVCGTDGVTYDNECLLCAHKVEQGA |
| | | SVDKRHDGGCRKELAAVSVDCSEYPKPDCTAEDRPLCGSDNKTYGNKCNFCNAVVESNGTLTLS |
| | | HFGKC |
| rOVD [gallus] coding | SEQ ID | MRFPSIFTAVLFAASSALAAPVNTTTEDETAQIPAEAVIGYSDLEGDFDVAVLPFSNSTNNGLL |
| sequence containing an | NO: 39 | FINTTIASIAAKEEGVSLEKREAEAAEVDCSRFPNATDKEGKDVLVCNKDLRPICGTDGVTYTND |
| alpha mating factor | | CLLCAYSIEFGTNISKEHDGECKETVPMNCSSYANTTSEDGKVMVLCNRAFNPVCGTDGVTYDNE |
| signal sequence | | CLLCAHKVEQGASVDKRHDGGCRKELAAVSVDCSEYPKPDCTAEDRPLCGSDNKTYGNKCNFCN |
| (bolded) as expressed in | | AVVESNGTLTLSHFGKC |
| pichia | | |
| Turkey vulture OVD | SEQ ID | MRFPSIFTAVLFAASSALAAPVNTTTEDETAQIPAEAVIGYSDLEGDFDVAVLPFSNSTNNGLL |
| coding sequence | NO: 40 | FINTTIASIAAKEEGVSLEKREAEAVEVDCSTYPNTTNEEGKEVLVCTKILSPICGTDGVTYSNECL |
| containing secretion | | LCAYNIEYGTNVSKDHDGECKEFVPVDCSRYPNTTNEDGKVVLLCNKDLSPICGTDGVTYDNECL |
| signals as expressed in | | LCARNLEPGTSVGKKYDGECKKEIATVDCSDYPKPVCSLEYMPLCGSDSKTYSNKCNFCNAVVDS |
| pichia | | NGTLTLSHFGKC |
| bolded is an alpha | | |
| mating factor signal | | |
| sequence | | |
| Turkey vulture OVD in | SEQ ID | EAEAVEVDCSTYPNTTNEEGKEVLVCTKILSPICGTDGVTYSNECLLCAYNIEYGTNVSKDHDGEC |
| secreted form expressed | NO: 41 | KEFVPVDCSRYPNTTNEDGKVVLLCNKDLSPICGTDGVTYDNECLLCARNLEPGTSVGKKYDGEC |
| in Pichia | | KKEIATVDCSDYPKPVCSLEYMPLCGSDSKTYSNKCNFCNAVVDSNGTLTLSHFGKC |
| Humming bird | SEQ ID | MTMAGVFVLLSFILCCFPDTAFGVEVDCSIYPNTTSEEGKEVLVCTETLSPICGSDGVTYNNECQL |
| OVD (native sequence) | NO: 42 | CAYNVEYGTNVSKDHDGECKEIVPVDCSRYPNTTEEGRVVMLCNKALSPVCGTDGVTYDNECLL |
| bolded is the native | | CARNLESGTSVGKKFDGECKKEIATVDCTDYPKPVCSLDYMPLCGSDSKTYSNKCNFCNAVMDSN |
| signal sequence | | GTLTLNHFGKC |
| Humming bird OVD | SEQ ID | MRFPSIFTAVLFAASSALAAPVNTTTEDETAQIPAEAVIGYSDLEGDFDVAVLPFSNSTNNGLL |
| coding sequence as | NO: 43 | FINTTIASIAAKEEGVSLDKREAEAVEVDCSIYPNTTSEEGKEVLVCTETLSPICGSDGVTYNNECQ |
| expressed in Pichia | | LCAYNVEYGTNVSKDHDGECKEIVPVDCSRYPNTTEEGRVVMLCNKALSPVCGTDGVTYDNECL |
| bolded is an alpha | | LCARNLESGTSVGKKFDGECKKEIATVDCTDYPKPVCSLDYMPLCGSDSKTYSNKCNFCNAVMDS |
| mating factor signal | | NGTLTLNHFGKC |
| sequence | | |
| Humming bird OVD in | SEQ ID | EAEAVEVDCSIYPNTTSEEGKEVLVCTETLSPICGSDGVTYNNECQLCAYNVEYGTNVSKDHDGEC |
| secreted form from | NO: 44 | KEIVPVDCSRYPNTTEEGRVVMLCNKALSPVCGTDGVTYDNECLLCARNLESGTSVGKKFDGECK |
| Pichia | | KEIATVDCTDYPKPVCSLDYMPLCGSDSKTYSNKCNFCNAVMDSNGTLTLNHFGKC |
| Chicken Ovalbumin | SEQ ID | MRFPSIFTAVLFAASSALAAPVNTTTEDETAQIPAEAVIGYSDLEGDFDVAVLPFSNSTNNGLL |
| with bolded signal | NO: 45 | FINTTIASIAAKEEGVSLDKREAEAGSIGAASMEFCFDVFKELKVHHANENIFYCPIAIMSALAMV |
| sequence | | YLGAKDSTRTQINKVVRFDKLPGFGDSIEAQCGTSVNVHSSLRDILNQITKPNDVYSFSLASRLYAE |
| | | ERYPILPEYLQCVKELYRGGLEPINFQTAADQARELINSWVESQTNGIIRNVLQPSSVDSQTAMVLV |
| | | NAIVFKGLWEKAFKDEDTQAMPFRVTEQESKPVQMMYQIGLFRVASMASEKMKILELPFASGTMS |
| | | MLVLLPDEVSGLEQLESIINFEKLTEWTSSNVMEERKIKVYLPRMKMEEKYNLTSVLMAMGITDVF |
| | | SSSANLSGISSAESLKISQAVHAAHAEINEAGREVVGSAEAGVDAASVSEEFRADHPFLFCIKHIATN |
| | | AVLFFGRCVSP |
| Chicken OVA sequence | SEQ ID | EAEAGSIGAASMEFCFDVFKELKVHHANENIFYCPIAIMSALAMVYLGAKDSTRTQINKVVRFDKL |
| as secreted from pichia | NO: 46 | PGFGDSIEAQCGTSVNVHSSLRDILNQITKPNDVYSFSLASRLYAEERYPILPEYLQCVKELYRGGLE |
| | | PINFQTAADQARELINSWVESQTNGIIRNVLQPSSVDSQTAMVLVNAIVFKGLWEKAFKDEDTQA |
| | | MPFRVTEQESKPVQMMYQIGLFRVASMASEKMKILELPFASGTMSMLVLLPDEVSGLEQLESIINF |
| | | EKLTEWTSSNVMEERKIKVYLPRMKMEEKYNLTSVLMAMGITDVFSSSANLSGISSAESLKISQAV |
| | | HAAHAEINEAGREVVGSAEAGVDAASVSEEFRADHPFLFCIKHIATNAVLFFGRCVSP |
| Predicted Ovalbumin | SEQ ID | MRVPAQLLGLLLLWLPGARCGSIGAASMEFCFDVFKELKVHHANENIFYCPIAIMSALAMVYLGA |
| [Achromobacter | NO: 47 | KDSTRTQINKVVRFDKLPGFGDSIEAQCGTSVNVHSSLRDILNQITKPNDVYSFSLASRLYAEERYPI |
| denitrificans] | | LPEYLQCVKELYRGGLEPINFQTAADQARELINSWVESQTNGIIRNVLQPSSVDSQTAMVLVNAIVF |
| | | KGLWEKAFKDEDTQAMPFRVTEQESKPVQMMYQIGLFRVASMASEKMKILELPFASGTMSMLVL |
| | | LPDEVSGLEQLESIINFEKLTEWTSSNVMEERKIKVYLPRMKMEEKYNLTSVLMAMGITDVFSSSA |
| | | NLSGISSAESLKISQAVHAAHAEINEAGREVVGSAEAGVDAASVSEEFRADHPFLFCIKHIATNAVL |
| | | FFGRCVSPLEIKRAAAHHHHHH |
| OLLAS epitope-tagged | SEQ ID | MTSGFANELGPRLMGKLTMGSIGAASMEFCFDVFKELKVHHANENIFYCPIAIMSALAMVYLGAK |
| ovalbumin | NO: 48 | DSTRTQINKVVRFDKLPGFGDSIEAQCGTSVNVHSSLRDILNQITKPNDVYSFSLASRLYAEERYPIL |
| | | PEYLQCVKELYRGGLEPINFQTAADQARELINSWVESQTNGIIRNVLQPSSVDSQTAMVLVNAIVF |
| | | KGLWEKTFKDEDTQAMPFRVTEQESKPVQMMYQIGLFRVASMASEKMKILELPFASGTMSMLVL |
| | | LPDEVSGLEQLESIINFEKLTEWTSSNVMEERKIKVYLPRMKMEEKYNLTSVLMAMGITDVFSSSA |
| | | NLSGISSAESLKISQAVHAAHAEINEAGREVVGSAEAGVDAASVSEEFRADHPFLFCIKHIATNAVL |
| | | FFGRCVSPSR |
| Serpin family protein | SEQ ID | MGGRRVRWEVYISRAGYVNRQIAWRRHHRSLTMRVPAQLLGLLLLWLPGARCGSIGAASMEFCF |
| [Achromobacter | NO: 49 | DVFKELKVHHANENIFYCPIAIMSALAMVYLGAKDSTRTQINKVVRFDKLPGFGDSIEAQCGTSVN |
| denitrificans] | | VHSSLRDILNQITKPNDVYSFSLASRLYAEERYPILPEYLQCVKELYRGGLEPINFQTAADQARELIN |
| | | SWVESQTNGIIRNVLQPSSVDSQTAMVLVNAIVFKGLWEKAFKDEDTQAMPFRVTEQESKPVQM |
| | | MYQIGLFRVASMASEKMKILELPFASGTMSMLVLLPDEVSGLEQLESIINFEKLTEWTSSNVMEER |
| | | KIKVYLPRMKMEEKYNLTSVLMAMGITDVFSSSANLSGISSAESLKISQAVHAAHAEINEAGREVV |
| | | GSAEAGVDAASVSEEFRADHPFLFCIKHIATNAVLFFGRCVSPLEIKRAAAHHHHHH |
| PREDICTED: | SEQ ID | MGSIGAVSMEFCFDVFKELKVHHANENIFYSPFTIISALAMVYLGAKDSTRTQINKVVRFDKLPGFG |
| ovalbumin isoform X1 | NO: 50 | DSVEAQCGTSVNVHSSLRDILNQITKPNDVYSFSLASRLYAEETYPILPEYLQCVKELYRGGLESINF |
| [Meleagris gallopavo] | | QTAADQARGLINSWVESQTNGMIKNVLQPSSVDSQTAMVLVNAIVFKGLWEKAFKDEDTQAIPFR |
| | | VTEQESKPVQMMYQIGLFKVASMASEKMKILELPFASGTMSMWVLLPDEVSGLEQLETTISFEKM |
| | | TEWISSNIMEERRIKVYLPRMKMEEKYNLTSVLMAMGITDLFSSSANLSGISSAGSLKISQAVHAAY |
| | | AEIYEAGREVIGSAEAGADATSVSEEFRVDHPFLYCIKHNLTNSILFFGRCISP |
| Ovalbumin precursor | SEQ ID | MGSIGAVSMEFCFDVFKELKVHHANENIFYSPFTIISALAMVYLGAKDSTRTQINKVVRFDKLPGFG |
| [Meleagris gallopavo] | NO: 51 | DSVEAQCGTSVNVHSSLRDILNQITKPNDVYSFSLASRLYAEETYPILPEYLQCVKELYRGGLESINF |
| | | QTAADQARGLINSWVESQTNGMIKNVLQPSSVDSQTAMVLVNAIVFKGLWEKAFKDEDTQAIPFR |
| | | VTEQESKPVQMMYQIGLFKVASMASEKMKILELPFASGTMSMWVLLPDEVSGLEQLETTISFEKM |
| | | TEWISSNIMEERRIKVYLPRMKMEEKYNLTSVLMAMGITDLFSSSANLSGISSAGSLKISQAAHAAY |
| | | AEIYEAGREVIGSAEAGADATSVSEEFRVDHPFLYCIKHNLTNSILFFGRCISP |
| Hypothetical protein | SEQ ID | YYRVPCMVLCTAFHPYIFIVLLFALDNSEFTMGSIGAVSMEFCFDVFKELRVHHPNENIFFCPFAIMS |
| [Bambusicola | NO: 52 | AMAMVYLGAKDSTRTQINKVIRFDKLPGFGDSTEAQCGKSANVHSSLKDILNQITKPNDVYSFSLA |
| thoracicus] | | SRLYADETYSIQSEYLQCVNELYRGGLESINFQTAADQARELINSWVESQTNGIIRNVLQPSSVDSQ |
| | | TAMVLVNAIVFRGLWEKAFKDEDTQTMPFRVTEQESKPVQMMYQIGSFKVASMASEKMKILELP |
| | | LASGTMSMLVLLPDEVSGLEQLETTISFEKLTEWTSSNVMEERKIKVYLPRMKMEEKYNLTSVLM |
| | | AMGITDLFRSSANLSGISLAGNLKISQAVHAAHAEINEAGRKAVSSAEAGVDATSVSEEFRADRPFL |
| | | FCIKHIATKVVFFFGRYTSP |
| Egg albumin | SEQ ID | MGSIGAASMEFCFDVFKELKVHHANDNMLYSPFAILSTLAMVFLGAKDSTRTQINKVVHFDKLPG |
| | NO: 53 | FGDSIEAQCGTSVNVHSSLRDILNQITKQNDAYSFSLASRLYAQETYTVVPEYLQCVKELYRGGLES |
| | | VNFQTAADQARGLINAWVESQTNGIIRNILQPSSVDSQTAMVLVNAIAFKGLWEKAFKAEDTQTIP |
| | | FRVTEQESKPVQMMYQIGSFKVASMASEKMKILELPFASGTMSMLVLLPDDVSGLEQLESIISFEKL |
| | | TEWTSSSIMEERKVKVYLPRMKMEEKYNLTSLLMAMGITDLFSSSANLSGISSVGSLKISQAVHAA |
| | | HAEINEAGRDVVGSAEAGVDATEEFRADHPFLFCVKHIETNAILLFGRCVSP |
| Ovalbumin isoform X2 | SEQ ID | MASIGAVSTEFCVDVYKELRVHHANENIFYSPFTIISTLAMVYLGAKDSTRTQINKVVRFDKLPGFG |
| [Numida meleagris] | NO: 54 | DSIEAQCGTSVNVHSSLRDILNQITKPNDVYSFSLASRLYAEETYPILPEYLQCVKELYRGGLESINF |
| | | QTAADQARELINSWVESQTSGIIKNVLQPSSVNSQTAMVLVNAIYFKGLWERAFKDEDTQAIPFRV |
| | | TEQESKPVQMMSQIGSFKVASVASEKVKILELPFVSGTMSMLVLLPDEVSGLEQLESTISTEKLTEW |
| | | TSSSIMEERKIKVFLPRMRMEEKYNLTSVLMAMGMTDLFSSSANLSGISSAESLKISQAVHAAYAEI |
| | | YEAGREVVSSAEAGVDATSVSEEFRVDHPFLLCIKHNPTNSILFFGRCISP |
| Ovalbumin isoform X1 | SEQ ID | MALCKAFHPYIFIVLLFDVDNSAFTMASIGAVSTEFCVDVYKELRVHHANENIFYSPFTIISTLAMV |
| [Numida meleagris] | NO: 55 | YLGAKDSTRTQINKVVRFDKLPGFGDSIEAQCGTSVNVHSSLRDILNQITKPNDVYSFSLASRLYAE |
| | | ETYPILPEYLQCVKELYRGGLESINFQTAADQARELINSWVESQTSGIIKNVLQPSSVNSQTAMVLV |
| | | NAIYFKGLWERAFKDEDTQAIPFRVTEQESKPVQMMSQIGSFKVASVASEKVKILELPFVSGTMSM |
| | | LVLLPDEVSGLEQLESTISTEKLTEWTSSSIMEERKIKVFLPRMRMEEKYNLTSVLMAMGMTDLFSS |
| | | SANLSGISSAESLKISQAVHAAYAEIYEAGREVVSSAEAGVDATSVSEEFRVDHPFLLCIKHNPTNSI |
| | | LFFGRCISP |
| PREDICTED: | SEQ ID | MGSIGAASMEFCFDVFKELKVHHANDNMLYSPFAILSTLAMVFLGAKDSTRTQINKVVHFDKLPG |
| Ovalbumin isoform X2 | NO: 56 | FGDSIEAQCGTSANVHSSLRDILNQITKQNDAYSFSLASRLYAQETYTVVPEYLQCVKELYRGGLES |
| [Coturnix japonica] | | VNFQTAADQARGLINAWVESQTNGIIRNILQPSSVDSQTAMVLVNAIAFKGLWEKAFKAEDTQTIP |
| | | FRVTEQESKPVQMMHQIGSFKVASMASEKMKILELPFASGTMSMLVLLPDDVSGLEQLESTISFEK |
| | | LTEWTSSSIMEERKVKVYLPRMKMEEKYNLTSLLMAMGITDLFSSSANLSGISSVGSLKISQAVHA |
| | | AYAEINEAGRDVVGSAEAGVDATEEFRADHPFLFCVKHIETNAILLFGRCVSP |
| PREDICTED: | SEQ ID | MGLCTAFHPYIFIVLLFALDNSEFTMGSIGAASMEFCFDVFKELKVHHANDNMLYSPFAILSTLAM |
| ovalbumin isoform X1 | NO: 57 | VFLGAKDSTRTQINKVVHFDKLPGFGDSIEAQCGTSANVHSSLRDILNQITKQNDAYSFSLASRLYA |
| [Coturnix japonica] | | QETYTVVPEYLQCVKELYRGGLESVNFQTAADQARGLINAWVESQTNGIIRNILQPSSVDSQTAMV |
| | | LVNAIAFKGLWEKAFKAEDTQTIPFRVTEQESKPVQMMHQIGSFKVASMASEKMKILELPFASGT |
| | | MSMLVLLPDDVSGLEQLESTISFEKLTEWTSSSIMEERKVKVYLPRMKMEEKYNLTSLLMAMGIT |
| | | DLFSSSANLSGISSVGSLKISQAVHAAYAEINEAGRDVVGSAEAGVDATEEFRADHPFLFCVKHIET |
| | | NAILLFGRCVSP |
| Egg albumin | SEQ ID | MGSIGAASMEFCFDVFKELKVHHANDNMLYSPFAILSTLAMVFLGAKDSTRTQINKVVHFDKLPG |
| | NO: 58 | FGDSIEAQCGTSANVHSSLRDILNQITKQNDAYSFSLASRLYAQETYTVVPEYLQCVKELYRGGLES |
| | | VNFQTAADQARGLINAWVESQTNGIIRNILQPSSVDSQTAMVLVNAIAFKGLWEKAFKAEDTQTIP |
| | | FRVTEQESKPVQMMHQIGSFKVASMASEKMKILELPFASGTMSMLVLLPDDVSGLEQLESTISFEK |
| | | LTEWTSSSIMEERKVKVYLPRMKMEEKYNLTSLLMAMGITDLFSSSANLSGISSVGSLKIPQAVHA |
| | | AYAEINEAGRDVVGSAEAGVDATEEFRADHPFLFCVKHIETNAILLFGRCVSP |
| ovalbumin [Anas | SEQ ID | MGSIGAASTEFCFDVFRELRVQHVNENIFYSPFSIISALAMVYLGARDNTRTQIDKVVHFDKLPGFG |
| platyrhynchos] | NO: 59 | ESMEAQCGTSVSVHSSLRDILTQITKPSDNFSLSFASRLYAEETYAILPEYLQCVKELYKGGLESISF |
| | | QTAADQARELINSWVESQTNGIIKNILQPSSVDSQTTMVLVNAIYFKGMWEKAFKDEDTQAMPFR |
| | | MTEQESKPVQMMYQVGSFKVAMVTSEKMKILELPFASGMMSMFVLLPDEVSGLEQLESTISFEKL |
| | | TEWTSSTMMEERRMKVYLPRMKMEEKYNLTSVFMALGMTDLFSSSANMSGISSTVSLKMSEAVH |
| | | AACVEIFEAGRDVVGSAEAGMDVTSVSEEFRADHPFLFFIKHNPTNSILFFGRWMSP |
| PREDICTED: | SEQ ID | MGSIGAASTEFCFDVFRELKVQHVNENIFYSPLSIISALAMVYLGARDNTRTQIDQVVHFDKIPGFG |
| ovalbumin-like [Anser | NO: 60 | ESMEAQCGTSVSVHSSLRDILTEITKPSDNFSLSFASRLYAEETYTILPEYLQCVKELYKGGLESISFQ |
| cygnoides domesticus] | | TAADQARELINSWVESQTNGIIKNILQPSSVDSQTTMVLVNAIYFKGMWEKAFKDEDTQTMPFRM |
| | | TEQESKPVQMMYQVGSFKLATVTSEKVKILELPFASGMMSMCVLLPDEVSGLEQLETTISFEKLTE |
| | | WTSSTMMEERRMKVYLPRMKMEEKYNLTSVFMALGMTDLFSSSANMSGISSTVSLKMSEAVHA |
| | | ACVEIFEAGRDVVGSAEAGMDVTSVSEEFRADHPFLFFIKHNPSNSILFFGRWISP |
| PREDICTED: | SEQ ID | MGSIGAASTEFCFDVFKELKVQHVNENIFYSPLTIISALSMVYLGARENTRAQIDKVLHFDKMPGFG |
| Ovalbumin-like [Aquila | NO: 61 | DTIESQCGTSVSIHTSLKDMFTQITKPSDNYSLSFASRLYAEETYPILPEYLQCVKELYKGGLETISFQ |
| chrysaetos canadensis] | | TAAEQARELINSWVESQTNGMIKNILQPSSVDPQTKMVLVNAIYFKGVWEKAFKDEDTQEVPFRV |
| | | TEQESKPVQMMYQIGSFKVAVMASEKMKILELPYASGQLSMLVLLPDDVSGLEQLESAITFEKLM |
| | | AWTSSTTMEERKMKVYLPRMKIEEKYNLTSVLMALGVTDLFSSSANLSGISSAESLKISKAVHEAF |
| | | VEIYEAGSEVVGSTEAGMEVTSVSEEFRADHPFLFLIKHNPTNSILFFGRCFSP |
| PREDICTED: | SEQ ID | MGSIGAASTEFCFDVFKELKVQHVNENIFYSPLTIISALSMVYLGARENTRTQIDKVLHFDKMTGFG |
| Ovalbumin-like | NO: 62 | DTVESQCGTSVSIHTSLKDIFTQITKPSDNYSLSLASRLYAEETYPILPEYLQCVKELYKGGLETVSF |
| [Haliaeetus albicilla] | | QTAAEQARELINSWVESQTNGMIKNILQPSSVDPQTKMVLVNAIYFKGVWEKAFKDEDTQEVPFR |
| | | VTEQESKPVQMMYQIGSFKVAVMASEKMKILELPYASGQLSMLVLLPDDVSGLEQLESAITSEKL |
| | | MEWTSSTTMEERKMKVYLPRMKIEEKYNLTSVLMALGVTDLFSSSADLSGISSAESLKISKAVHEA |
| | | FVEIYEAGSEVVGSTEGGMEVTSVSEEFRADHPFLFLIKHKPTNSILFFGRCFSP |
| PREDICTED: | SEQ ID | MGSIGAASTEFCFDVFKELKVQHVNENIFYSPLTIISALSMVYLGARENTRTQIDKVLHFDKMTGFG |
| Ovalbumin-like | NO: 63 | DTVESQCGTSVSIHTSLKDIFTQITKPSDNYSLSLASRLYAEETYPILPEYLQCVKELYKGGLETVSF |
| [Haliaeetus | | QTAAEQARELINSWVESQTNGMIKNILQPSSVDPQTKMVLVNAIYFKGVWEKAFKDEDTQEVPFR |
| leucocephalus] | | VTEQESKPVQMMYQIGSFKVAVMASEKMKILELPYASGQLSMLVLLPDDVSGLEQLESAITSEKL |
| | | MEWTSSTTMEERKMKVYLPRMKIEEKYNLTSVLMALGVTDLFSSSADLSGISSAESLKISKAVHEA |
| | | FVEIYEAGSEVVGSTEGGMEVTSFSEEFRADHPFLFLIKHKPTNSILFFGRCFSP |
| PREDICTED: | SEQ ID | MGSIGAASTEFCFDVFKELKVQHVNENIFYSPLSIISALSMVYLGARENTRAQIDKVVHFDKITGFG |
| Ovalbumin [Fulmarus | NO: 64 | ETIESQCGTSVSVHTSLKDMFTQITKPSDNYSLSFASRLYAEETYPILPEYLQCVKELYKGGLETTSF |
| glacialis] | | QTAADQARELINSWVESQTNGMIKNILQPGSVDPQTEMVLVNAIYFKGMWEKAFKDEDTQAVPF |
| | | RMTEQESKTVQMMYQIGSFKVAVMASEKMKILELPYASGELSMLVMLPDDVSGLEQLETAITFEK |
| | | LMEWTSSNMMEERKMKVYLPRMKMEEKYNLTSVLMALGVTDLFSSSANLSGISSAESLKMSEAV |
| | | HEAFVEIYEAGSEVVGSTGAGMEVTSVSEEFRADHPFLFLIKHNPTNSILFFGRCFSP |
| PREDICTED: | SEQ ID | MGSIGAASTEFCFDVFKELRVQHVNENVCYSPLIIISALSLVYLGARENTRAQIDKVVHFDKITGFG |
| Ovalbumin-like | NO: 65 | ESIESQCGTSVSVHTSLKDMFNQITKPSDNYSLSVASRLYAEERYPILPEYLQCVKELYKGGLESISF |
| [Chlamydotis | | QTAADQAREAINSWVESQTNGMIKNILQPSSVDPQTEMVLVNAIYFKGMWQKAFKDEDTQAVPF |
| macqueenii] | | RISEQESKPVQMMYQIGSFKVAVMAAEKMKILELPYASGELSMLVLLPDEVSGLEQLENAITVEKL |
| | | MEWTSSSPMEERIMKVYLPRMKIEEKYNLTSVLMALGITDLFSSSANLSGISAEESLKMSEAVHQA |
| | | FAEISEAGSEVVGSSEAGIDATSVSEEFRADHPFLFLIKHNATNSILFFGRCFSP |
| PREDICTED: | SEQ ID | MGSISAASTEFCFDVFKELKVQHVNENIFYSPLSIISALSMVYLGARENTRAQIEKVVHFDKITGFGE |
| Ovalbumin like | NO: 66 | SIESQCSTSVSVHTSLKDMFTQITKPSDNYSLSFASRFYAEETYPILPEYLQCVKELYKGGLETINFRT |
| [Nipponia nippon] | | AADQARELINSWVESQTNGMIKNILQPGSVDPQTDMVLVNAIYFKGMWEKAFKDEDTQALPFRV |
| | | TEQESKPVQMMYQIGSFKVAVLASEKVKILELPYASGQLSMLVLLPDDVSGLEQLETAITVEKLME |
| | | WTSSNNMEERKIKVYLPRIKIEEKYNLTSVLMALGITDLFSSSANLSGISSAESLKVSEAIHEAFVEIY |
| | | EAGSEVAGSTEAGIEVTSVSEEFRADHPFLFLIKHNATNSILFFGRCFSP |
| PREDICTED: | SEQ ID | MVSIGAASTEFCFDVFKELKVQHVNENIFYSPLSIISALSMVYLGARENTRAQIDKVVHFDKITGFEE |
| Ovalbumin-like | NO: 67 | TIESQCSTSVSVHTSLKDMFTQITKPSDNYSLSFASRLYAEETYPILPEYLQCVKELYKGGLETISFQT |
| isoform X2 [Gavia | | AADQARELINSWVESQTDGMIKNILQPGSVDPQTEMVLVNAIYFKGMWEKAFKDEDTQAVPFRM |
| stellata] | | TEQESKPVQMMYQIGSFKVAVMASEKMKILELPYASGGMSMLVMLPDDVSGLEQLETAITFEKL |
| | | MEWTSSNMMEERKMKVYLPRMKMEEKYNLTSVLMALGMTDLFSSSANLSGISSAESLKMSEAV |
| | | HEAFVEIYEAGSEAVGSTGAGMEVTSVSEEFRADHPFLFLIKHNPTNSILFFGRCFSP |
| PREDICTED: | SEQ ID | MGSIGAASTEFCFDVFKELKVQHVNENIFYSPLSIISALSMVYLGARENTRAQIDKVVHFDKITGFG |
| Ovalbumin [Pelecanus | NO: 68 | EPIESQCGISVSVHTSLKDMITQITKPSDNYSLSFASRLYAEETYPILPEYLQCVKELYKGGLETISFQ |
| crispus] | | TAADQARELINSWVENQTNGMIKNILQPGSVDPQTEMVLVNAVYFKGMWEKAFKDEDTQAVPFR |
| | | MTEQESKPVQMMYQIGSFKVAVMASEKIKILELPYASGELSMLVLLPDDVSGLEQLETAITLDKLT |
| | | EWTSSNAMEERKMKVYLPRMKIEKKYNLTSVLIALGMTDLFSSSANLSGISSAESLKMSEAIHEAF |
| | | LEIYEAGSEVVGSTEAGMEVTSVSEEFRADHPFLFLIKHNPTNSILFFGRCLSP |
| PREDICTED: | SEQ ID | MGSIGAASTEFCFDVFKELKVQHVNENIFYSPLTIISALSMVYLGARENTRAQIDKVVHFDKIPGFG |
| Ovalbumin-like | NO: 69 | DTTESQCGTSVSVHTSLKDMFTQITKPSDNYSVSFASRLYAEETYPILPEFLECVKELYKGGLESISF |
| [Charadrius vociferus] | | QTAADQARELINSWVESQTNGMIKNILQPGSVDSQTEMVLVNAIYFKGMWEKAFKDEDTQTVPFR |
| | | MTEQETKPVQMMYQIGTFKVAVMPSEKMKILELPYASGELCMLVMLPDDVSGLEELESSITVEKL |
| | | MEWTSSNMMEERKMKVFLPRMKIEEKYNLTSVLMALGMTDLFSSSANLSGISSAEPLKMSEAVHE |
| | | AFIEIYEAGSEVVGSTGAGMEITSVSEEFRADHPFLFLIKHNPTNSILFFGRCVSP |
| PREDICTED: | SEQ ID | MGSIGAVSTEFCFDVFKELKVQHVNENIFYSPLSIISALSMVYLGARENTRAQIDKVVHFDKITGSG |
| Ovalbumin-like | NO: 70 | ETIEAQCGTSVSVHTSLKDMFTQITKPSENYSVGFASRLYADETYPIIPEYLQCVKELYKGGLEMISF |
| [Eurypyga helias] | | QTAADQARELINSWVESQTNGMIKNILQPGSVDPQTEMILVNAIYFKGVWEKAFKDEDTQAVPFR |
| | | MTEQESKPVQMMYQFGSFKVAAMAAEKMKILELPYASGALSMLVLLPDDVSGLEQLESAITFEKL |
| | | MEWTSSNMMEEKKIKVYLPRMKMEEKYNFTSVLMALGMTDLFSSSANLSGISSADSLKMSEVVH |
| | | EAFVEIYEAGSEVVGSTGSGMEAASVSEEFRADHPFLFLIKHNPTNSILFFGRCFSP |
| PREDICTED: | SEQ ID | MVSIGAASTEFCFDVFKELKVQHVNENIFYSPLSIISALSMVYLGARENTRAQIDKVVHFDKITGFEE |
| Ovalbumin-like | NO: 71 | TIESQVQKKQCSTSVSVHTSLKDMFTQITKPSDNYSLSFASRLYAEETYPILPEYLQCVKELYKGGL |
| isoform X1 [Gavia | | ETISFQTAADQARELINSWVESQTDGMIKNILQPGSVDPQTEMVLVNAIYFKGMWEKAFKDEDTQ |
| stellata] | | AVPFRMTEQESKPVQMMYQIGSFKVAVMASEKMKILELPYASGGMSMLVMLPDDVSGLEQLETA |
| | | ITFEKLMEWTSSNMMEERKMKVYLPRMKMEEKYNLTSVLMALGMTDLFSSSANLSGISSAESLK |
| | | MSEAVHEAFVEIYEAGSEAVGSTGAGMEVTSVSEEFRADHPFLFLIKHNPTNSILFFGRCFSP |
| PREDICTED: | SEQ ID | MGSIGAASGEFCFDVFKELKVQHVNENIFYSPLSIISALSMVYLGARENTRAQIDKVVHFDKIIGFGE |
| Ovalbumin-like | NO: 72 | SIESQCGTSVSVHTSLKDMFAQITKPSDNYSLSFASRLYAEETFPILPEYLQCVKELYKGGLETLSFQ |
| [Egretta garzetta] | | TAADQARELINSWVESQTNGMIKDILQPGSVDPQTEMVLVNAIYFKGVWEKAFKDEDTQTVPFRM |
| | | TEQESKPVQMMYQIGSFKVAVVAAEKIKILELPYASGALSMLVLLPDDVSSLEQLETAITFEKLTE |
| | | WTSSNIMEERKIKVYLPRMKIEEKYNLTSVLMDLGITDLFSSSANLSGISSAESLKVSEAIHEAIVDIY |
| | | EAGSEVVGSSGAGLEGTSVSEEFRADHPFLFLIKHNPTSSILFFGRCFSP |
| PREDICTED: | SEQ ID | MGSIGAASTEFCFDVFKELKVQHVNENIFYSPLSIISALSMVYLGARENTRAQIDKVVHFDKITGSG |
| Ovalbumin-like | NO: 73 | EAIESQCGTSVSVHISLKDMFTQITKPSDNYSLSFASRLYAEETYPILPEYLQCVKELYKEGLATISFQ |
| [Balearica regulorum | | TAADQAREFINSWVESQTNGMIKNILQPGSVDPQTQMVLVNAIYFKGVWEKAFKDEDTQAVPFR |
| gibbericeps] | | MTKQESKPVQMMYQIGSFKVAVMASEKMKILELPYASGQLSMLVMLPDDVSGLEQIENAITFEKL |
| | | MEWTNPNMMEERKMKVYLPRMKMEEKYNLTSVLMALGMTDLFSSSANLSGISSAESLKMSEAV |
| | | HEAFVEIYEAGSEVVGSTGAGIEVTSVSEEFRADHPFLFLIKHNPTNSILFFGRCFSP |
| PREDICTED: | SEQ ID | MGSIGEASTEFCIDVFRELKVQHVNENIFYSPLSIISALSMVYLGARENTRAQIDQVVHFDKITGFGD |
| Ovalbumin-like [Nestor | NO: 74 | TVESQCGSSLSVHSSLKDIFAQITQPKDNYSLNFASRLYAEETYPILPEYLQCVKELYKGGLETISFQ |
| notabilis] | | TAADQARELINSWVESQTNGMIKNILQPSSVDPQTEMVLVNAIYFKGVWEKAFKDEETQAVPFRIT |
| | | EQENRPVQIMYQFGSFKVAVVASEKIKILELPYASGQLSMLVLLPDEVSGLEQLENAITFEKLTEWT |
| | | SSDIMEEKKIKVFLPRMKIEEKYNLTSVLVALGIADLFSSSANLSGISSAESLKMSEAVHEAFVEIYE |
| | | AGSEVVGSSGAGIEAASDSEEFRADHPFLFLIKHKPTNSILFFGRCFSP |
| PREDICTED: | SEQ ID | MGSIGAASTEFCFDIFNELKVQHVNENIFYSPLSIISALSMVYLGARENTKAQIDKVVHFDKITGFGE |
| Ovalbumin-like | NO: 75 | SIESQCSTSASVHTSFKDMFTQITKPSDNYSLSFASRLYAEETYPILPEYSQCVKELYKGGLESISFQT |
| [Pygoscelis adeliae] | | AADQARELINSWVESQTNGMIKNILQPGSVDPQTELVLVNAIYFKGTWEKAFKDKDTQAVPFRVT |
| | | EQESKPVQMMYQIGSYKVAVIASEKMKILELPYASGELSMLVLLPDDVSGLEQLETAITFEKLMEW |
| | | TSSNMMEERKVKVYLPRMKIEEKYNLTSVLMALGMTDLFSPSANLSGISSAESLKMSEAIHEAFVE |
| | | IYEAGSEVVGSTEAGMEVTSVSEEFRADHPFLFLIKCNLTNSILFFGRCFSP |
| Ovalbumin-like | SEQ ID | MGSISTASTEFCFDVFKELKVQHVNENIFYSPLSIISALSMVYLGARENTRAQIEKVVHFDKITGFGE |
| [Athene cunicularia] | NO: 76 | SIESQCGTSVSVHTSLKDMLIQISKPSDNYSLSFASKLYAEETYPILPEYLQCVKELYKGGLESINFQT |
| | | AADQARQLINSWVESQTNGMIKDILQPSSVDPQTEMVLVNAIYFKGIWEKAFKDEDTQEVPFRITE |
| | | QESKPVQMMYQIGSFKVAVIASEKIKILELPYASGELSMLIVLPDDVSGLEQLETAITFEKLIEWTSP |
| | | SIMEERKTKVYLPRMKIEEKYNLTSVLMALGMTDLFSPSANLSGISSAESLKMSEAIHEAFVEIYEA |
| | | GSEVVGSAEAGMEATSVSEFRVDHPFLFLIKHNPANIILFFGRCVSP |
| PREDICTED: | SEQ ID | MGSIGAASTEFCFDVFKELKVQHVNENIFYSPLTIISALSLVYLGARENTRAQIDKVFHFDKISGFGE |
| Ovalbumin-like | NO: 77 | TTESQCGTSVSVHTSLKEMFTQITKPSDNYSVSFASRLYAEDTYPILPEYLQCVKELYKGGLETISFQ |
| [Calidris pugnax] | | TAADQAREVINSWVESQTNGMIKNILQPGSVDSQTEMVLVNAIYFKGMWEKAFKDEDTQTMPFRI |
| | | TEQERKPVQMMYQAGSFKVAVMASEKMKILELPYASGEFCMLIMLPDDVSGLEQLENSFSFEKLM |
| | | EWTTSNMMEERKMKVYIPRMKMEEKYNLTSVLMALGMTDLFSSSANLSGISSAETLKMSEAVHE |
| | | AFMEIYEAGSEVVGSTGSGAEVTGVYEEFRADHPFLFLVKHKPTNSILFFGRCVSP |
| PREDICTED: | SEQ ID | MGSIGAASTEFCFDIFNELKVQHVNENIFYSPLSIISALSMVYLGARENTKAQIDKVVHFDKITGFGE |
| Ovalbumin | NO: 78 | TIESQCSTSVSVHTSLKDTFTQITKPSDNYSLSFASRLYAEETYPILPEYSQCVKELYKGGLETISFQT |
| [Aptenodytes forsteri] | | AADQARELINSWVESQTNGMIKNILQPGSVDPQTELVLVNAIYFKGTWEKAFKDKDTQAVPFRVT |
| | | EQESKPVQMMYQIGSYKVAVIASEKMKILELPYASRELSMLVLLPDDVSGLEQLETAITFEKLMEW |
| | | TSSNMMEERKVKVYLPRMKIEEKYNLTSVLMALGMTDLFSPSANLSGISSAESLKMSEAVHEAFV |
| | | EIYEAGSEVVGSTGAGMEVTSVSEEFRADHPFLFLIKCNPTNSILFFGRCFSP |
| PREDICTED: | SEQ ID | MGSISAASAEFCLDVFKELKVQHVNENIFYSPLSIISALSMVYLGARENTRAQIDKVVHFDKITGSG |
| Ovalbumin-like | NO: 79 | ETIEFQCGTSANIHPSLKDMFTQITRLSDNYSLSFASRLYAEERYPILPEYLQCVKELYKGGLETISFQ |
| [Pterocles gutturalis] | | TAADQARELINSWVESQTNGMIKNILQPGSVNPQTEMVLVNAIYFKGLWEKAFKDEDTQTVPFRM |
| | | TEQESKPVQMMYQVGSFKVAVMASDKIKILELPYASGELSMLVLLPDDVTGLEQLETSITFEKLME |
| | | WTSSNVMEERTMKVYLPHMRMEEKYNLTSVLMALGVTDLFSSSANLSGISSAESLKMSEAVHEAF |
| | | VEIYESGSQVVGSTGAGTEVTSVSEEFRVDHPFLFLIKHNPTNSILFFGRCFSP |
| Ovalbumin-like [Falco | SEQ ID | MGSIGAASVEFCFDVFKELKVQHVNENIFYSPLSIISALSMVYLGARENTKAQIDKVVHFDKIAGFG |
| peregrinus] | NO: 80 | EAIESQCVTSASIHSLKDMFTQITKPSDNYSLSFASRLYAEEAYSILPEYLQCVKELYKGGLETISFQT |
| | | AADQARDLINSWVESQTNGMIKNILQPGAVDLETEMVLVNAIYFKGMWEKAFKDEDTQTVPFRM |
| | | TEQESKPVQMMYQVGSFKVAVMASDKIKILELPYASGQLSMVVVLPDDVSGLEQLEASITSEKLM |
| | | EWTSSSIMEEKKIKVYFPHMKIEEKYNLTSVLMALGMTDLFSSSANLSGISSAEKLKVSEAVHEAFV |
| | | EISEAGSEVVGSTEAGTEVTSVSEEFKADHPFLFLIKHNPTNSILFFGRCFSP |
| PREDICTED: | SEQ ID | MGSIGAASSEFCFDIFKELKVQHVNENIFYSPLSIISALSMVYLGARENTRAQIDKVVPFDKITASGE |
| Ovalbumin-like | NO: 81 | SIESQCSTSVSVHTSLKDIFTQITKSSDNHSLSFASRLYAEETYPILPEYLQCVKELYEGGLETISFQT |
| isoform X2 | | AADQARELINSWIESQTNGRIKNILQPGSVDPQTEMVLVNAIYFKGMWEKAFKDEDTQAVPFRMTE |
| [Phalacrocorax carbo] | | QESKPVQVMHQIGSFKVAVLASEKIKILELPYASGELSMLVLLPDDVSGLEQLETAITFEKLMEWTS |
| | | PNIMEERKIKVFLPRMKIEEKYNLTSVLMALGITDLFSPLANLSGISSAESLKMSEAIHEAFVEISEAG |
| | | SEVIGSTEAEVEVTNDPEEFRADHPFLFLIKHNPTNSILFFGRCFSP |
| PREDICTED: | SEQ ID | MGSIGAASTEFCFDVFKELKAQYVNENIFYSPMTIITALSMVYLGSKENTRAQIAKVAHFDKITGFG |
| Ovalbumin-like | NO: 82 | ESIESQCGASASIQFSLKDLFTQITKPSGNHSLSVASRIYAEETYPILPEYLECMKELYKGGLETINFQ |
| [Merops nubicus] | | TAANQARELINSWVERQTSGMIKNILQPSSVDSQTEMVLVNAIYFRGLWEKAFKVEDTQATPFRIT |
| | | EQESKPVQMMHQIGSFKVAVVASEKIKILELPYASGRLTMLVVLPDDVSGLKQLETTITFEKLMEW |
| | | TTSNIMEERKIKVYLPRMKIEEKYNLTSVLMALGLTDLFSSSANLSGISSAESLKMSEAVHEAFVEIY |
| | | EAGSEVVASAEAGMDATSVSEEFRADHPFLFLIKDNTSNSILFFGRCFSP |
| PREDICTED: | SEQ ID | MGSIGAASTEFCFDVFKELKGQHVNENIFFCPLSIVSALSMVYLGARENTRAQIVKVAHFDKIAGFA |
| Ovalbumin-like | NO: 83 | ESIESQCGTSVSIHTSLKDMFTQITKPSDNYSLNFASRLYAEETYPIIPEYLQCVKELYKGGLETISFQ |
| [Tauraco | | TAADQAREIINSWVESQTNGMIKNILRPSSVHPQTELVLVNAVYFKGTWEKAFKDEDTQAVPFRIT |
| erythrolophus] | | EQESKPVQMMYQIGSFKVAAVTSEKMKILEVPYASGELSMLVLLPDDVSGLEQLETAITAEKLIEW |
| | | TSSTVMEERKLKVYLPRMKIEEKYNLTTVLTALGVTDLFSSSANLSGISSAQGLKMSNAVHEAFVE |
| | | IYEAGSEVVGSKGEGTEVSSVSDEFKADHPFLFLIKHNPTNSIVFFGRCFSP |
| PREDICTED: | SEQ ID | MGSIGAASTEFCFDVFKELKVHHVNENILYSPLAIISALSMVYLGAKENTRDQIDKVVHFDKITGIG |
| Ovalbumin-like | NO: 84 | ESIESQCSTAVSVHTSLKDVFDQITRPSDNYSLAFASRLYAEKTYPILPEYLQCVKELYKGGLETIDF |
| [Cuculus canorus] | | QTAADQARQLINSWVEDETNGMIKNILRPSSVNPQTKIILVNAIYFKGMWEKAFKDEDTQEVPFRIT |
| | | EQETKSVQMMYQIGSFKVAEVVSDKMKILELPYASGKLSMLVLLPDDVYGLEQLETVITVEKLKE |
| | | WTSSIVMEERITKVYLPRMKIMEKYNLTSVLTAFGITDLFSPSANLSGISSTESLKVSEAVHEAFVEI |
| | | HEAGSEVVGSAGAGIEATSVSEEFKADHPFLFLIKHNPTNSILFFGRCFSP |
| Ovalbumin | SEQ ID | MGSIGAASTEFCLDVFKELKVQHVNENIFYSPLSIISALSMVYLGARENTRAQIDKVVHFDKITGFE |
| [Antrostomus | NO: 85 | DSIESQCGTSVSVHTSLKDMFTQITKPSDNYSVGFASRLYAAETYQILPEYSQCVKELYKGGLETIN |
| carolinensis] | | FQKAADQATELINSWVESQTNGMIKNILQPSSVDPQTQIFLVNAIYFKGMWQRAFKEEDTQAVPFR |
| | | ISEKESKPVQMMYQIGSFKVAVIPSEKIKILELPYASGLLSMLVILPDDVSGLEQLENAITLEKLMQW |
| | | TSSNMMEERKIKVYLPRMRMEEKYNLTSVFMALGITDLFSSSANLSGISSAESLKMSDAVHEASVE |
| | | IHEAGSEVVGSTGSGTEASSVSEEFRADHPYLFLIKHNPTDSIVFFGRCFSP |
| PREDICTED: | SEQ ID | MGSIGAASTEFCFDVFKELKFQHVDENIFYSPLTIISALSMVYLGARENTRAQIDKVVHFDKIAGFEE |
| Ovalbumin-like | NO: 86 | TVESQCGTSVSVHTSLKDMFAQITKPSDNYSLSFASRLYAEETYPILPEYLQCVKELYKGGLETISFQ |
| [Opisthocomus hoazin] | | TAADQARDLINSWVESQTNGMIKNILQPSSVGPQTELILVNAIYFKGMWQKAFKDEDTQEVPFRM |
| | | TEQQSKPVQMMYQTGSFKVAVVASEKMKILALPYASGQLSLLVMLPDDVSGLKQLESAITSEKLIE |
| | | WTSPSMMEERKIKVYLPRMKIEEKYNLTSVLMALGITDLFSPSANLSGISSAESLKMSQAVHEAFV |
| | | EIYEAGSEVVGSTGAGMEDSSDSEEFRVDHPFLFFIKHNPTNSILFFGRCFSP |
| PREDICTED: | SEQ ID | MGSIGPLSVEFCCDVFKELRIQHPRENIFYSPVTIISALSMVYLGARDNTKAQIEKAVHFDKIPGFGE |
| Ovalbumin-like | NO: 87 | SIESQCGTSLSIHTSLKDIFTQITKPSDNYTVGIASRLYAEEKYPILPEYLQCIKELYKGGLEPINFQTA |
| [Lepidothrix coronata] | | AEQARELINSWVESQTNGMIKNILQPSSVNPETDMVLVNAIYFKGLWEKAFKDEDIQTVPFRITEQE |
| | | SKPVQMMFQIGSFRVAEITSEKIRILELPYASGQLSLWVLLPDDISGLEQLETAITFENLKEWTSSTK |
| | | MEERKIKVYLPRMKIEEKYNLTSVLTSLGITDLFSSSANLSGISSAESLKVSSAFHEASVEIYEAGSK |
| | | VVGSTGAEVEDTSVSEEFRADHPFLFLIKHNPSNSIFFFGRCFSP |
| PREDICTED: | SEQ ID | MGSIGTASAEFCFDVFKELKVHHVNENIFYSPLSIISALSMVYLGARENTKTQMEKVIHFDKITGLG |
| Ovalbumin [Struthio | NO: 88 | ESMESQCGTGVSIHTALKDMLSEITKPSDNYSLSLASRLYAEQTYAILPEYLQCIKELYKESLETVSF |
| camelus australis] | | QTAADQARELINSWIESQTNGVIKNFLQPGSVDSQTELVLVNAIYFKGMWEKAFKDEDTQEVPFRI |
| | | TEQESRPVQMMYQAGSFKVATVAAEKIKILELPYASGELSMLVLLPDDISGLEQLETTISFEKLTEW |
| | | TSSNMMEDRNMKVYLPRMKIEEKYNLTSVLIALGMTDLFSPAANLSGISAAESLKMSEAIHAAYV |
| | | EIYEADSEIVSSAGVQVEVTSDSEEFRVDHPFLFLIKHNPTNSVLFFGRCISP |
| PREDICTED: | SEQ ID | MGSIGAVSTEFSCDVFKELRIHHVQENIFYSPVTIISALSMIYLGARDSTKAQIEKAVHFDKIPGFGES |
| Ovalbumin-like | NO: 89 | lESQCGTSLSIHTSIKDMFTKITKASDNYSIGIASRLYAEEKYPILPEYLQCVKELYKGGLESISFQTA |
| [Acanthisitta chloris] | | AEQAREIINSWVESQTNGMIKNILQPSSVDPQTDIVLVNAIYFKGLWEKAFRDEDTQTVPFKITEQE |
| | | SKPVQMMYQIGSFKVAEITSEKIKILEVPYASGQLSLWVLLPDDISGLEKLETAITFENLKEWTSSTK |
| | | MEERKIKVYLPRMKIEEKYNLTSVLTALGITDLFSSSANLSGISSAESLKVSEAFHEAIVEISEAGSKV |
| | | VGSVGAGVDDTSVSEEFRADHPFLFLIKHNPTSSIFFFGRCFSP |
| PREDICTED: | SEQ ID | MGSIGAASTEFCFDVFKELKVQHVNENIFYSPLSIISALSMVYLGARENTRAQIDKVVHFDKIAGFG |
| Ovalbumin-like [Tyto | NO: 90 | ESTESQCGTSVSAHTSLKDMSNQITKLSDNYSLSFASRLYAEETYPILPEYSQCVKELYKGGLESISF |
| alba] | | QTAAYQARELINAWVESQTNGMIKDILQPGSVDSQTKMVLVNAIYFKGIWEKAFKDEDTQEVPFR |
| | | MTEQETKPVQMMYQIGSFKVAVIAAEKIKILELPYASGQLSMLVILPDDVSGLEQLETAITFEKLTE |
| | | WTSASVMEERKIKVYLPRMSIEEKYNLTSVLIALGVTDLFSSSANLSGISSAESLRMSEAIHEAFVET |
| | | YEAGSTESGTEVTSASEEFRVDHPFLFLIKHKPTNSILFFGRCFSP |
| PREDICTED: | SEQ ID | MGSIGAASSEFCFDIFKELKVQHVNENIFYSPLSIISALSMVYLGARENTRAQIDKVVPFDKITASGE |
| Ovalbumin-like | NO: 91 | SIESQVQKIQCSTSVSVHTSLKDIFTQITKSSDNHSLSFASRLYAEETYPILPEYLQCVKELYEGGLE |
| isoform X1 | | TISFQTAADQARELINSWIESQTNGRIKNILQPGSVDPQTEMVLVNAIYFKGMWEKAFKDEDTQAVPF |
| [Phalacrocorax carbo] | | RMTEQESKPVQVMHQIGSFKVAVLASEKIKILELPYASGELSMLVLLPDDVSGLEQLETAITFEKLM |
| | | EWTSPNIMEERKIKVFLPRMKIEEKYNLTSVLMALGITDLFSPLANLSGISSAESLKMSEAIHEAFVEI |
| | | SEAGSEVIGSTEAEVEVTNDPEEFRADHPFLFLIKHNPTNSILFFGRCFSP |
| Ovalbumin-like [Pipra | SEQ ID | MGSIGPLSVEFCCDVFKELRIQHARENIFYSPVTIISALSMVYLGARDNTKAQIEKAVHFDKIPGFGE |
| filicauda] | NO: 92 | SIESQCGTSLSIHTSLKDIFTQITKPSDNYTVGIASRLYAEEKYPILPEYLQCIKELYKGGLEPISFQTA |
| | | AEQARELINSWVESQTNGIIKNILQPSSVNPETDMVLVNAIYFKGLWEKAFKDEGTQTVPFRITEQE |
| | | SKPVQMMFQIGSFRVAEIASEKIRILELPYASGQLSLWVLLPDDISGLEQLETAITFENLKEWTSSTK |
| | | MEERKIKVYLPRMKIEEKYNLTSVLTSLGITDLFSSSANLSGISSAERLKVSSAFHEASMEINEAGSK |
| | | VVGAGVDDTSVSEEFRVDRPFLFLIKHNPSNSIFFFGRCFSP |
| Ovalbumin [Dromaius | SEQ ID | MGSIGAASTEFCFDMFKELKVHHVNENIIYSPLSIISILSMVFLGARENTKTQMEKVIHFDKITGFGE |
| novaehollandiae] | NO: 93 | SLESQCGTSVSVHASLKDILSEITKPSDNYSLSLASKLYAEETYPVLPEYLQCIKELYKGSLETVSFQ |
| | | TAADQARELINSWVETQTNGVIKNFLQPGSVDPQTEMVLVDAIYFKGTWEKAFKDEDTQEVPFRIT |
| | | EQESKPVQMMYQAGSFKVATVAAEKMKILELPYASGELSMFVLLPDDISGLEQLETTISIEKLSEW |
| | | TSSNMMEDRKMKVYLPHMKIEEKYNLTSVLVALGMTDLFSPSANLSGISTAQTLKMSEAIHGAYV |
| | | EIYEAGSEMATSTGVLVEAASVSEEFRVDHPFLFLIKHNPSNSILFFGRCIFP |
| Chain A, Ovalbumin | SEQ ID | MGSIGAASTEFCFDMFKELKVHHVNENIIYSPLSIISILSMVFLGARENTKTQMEKVIHFDKITGFGE |
| | NO: 94 | SLESQCGTSVSVHASLKDILSEITKPSDNYSLSLASKLYAEETYPVLPEYLQCIKELYKGSLETVSFQ |
| | | TAADQARELINSWVETQTNGVIKNFLQPGSVDPQTEMVLVDAIYFKGTWEKAFKDEDTQEVPFRIT |
| | | EQESKPVQMMYQAGSFKVATVAAEKMKILELPYASGELSMFVLLPDDISGLEQLETTISIEKLSEW |
| | | TSSNMMEDRKMKVYLPHMKIEEKYNLTSVLVALGMTDLFSPSANLSGISTAQTLKMSEAIHGAYV |
| | | EIYEAGSEMATSTGVLVEAASVSEEFRVDHPFLFLIKHNPSNSILFFGRCIFPHHHHHH |
| Ovalbumin-like | SEQ ID | MGSIGPLSVEFCCDVFKELRIQHARENIFYSPVTIISALSMVYLGARDNTKAQIEKAVHFDKIPGFGE |
| [Corapipo altera] | NO: 95 | SIESQCGTSLSIHTSLKDIFTQITKPSDNYTVGIASRLYAEEKYPILPEYLQCIKELYKGGLEPISFQTA |
| | | AEQARELINSWVESQTNGMIKNILQPSAVNPETDMVLVNAIYFKGLWEKAFKDEGTQTVPFRITEQ |
| | | ESKPVQMMFQIGSFRVAEITSEKIRILELPYASGQLSLWVLLPDDISGLEQLETAITFENLKEWTSST |
| | | KMEERKIKVYLPRMKIEEKYNLTSVLTSLGITDLFSSSANLSGISSAERLKVSSAFHEASMEIYEAGS |
| | | KVVGSTGAGVDDTSVSEEFRVDRPFLFLIKHNPSNSIFFFGRCFSP |
| Ovalbumin-like protein | SEQ ID | MEDQRGNTGFTMGSIGAASTEFCIDVFRELRVQHVNENIFYSPLTIISALSMVYLGARENTRAQIDQ |
| [Amazona aestiva] | NO: 96 | VVHFDKIAGFGDTVESQCGSSPSVHNSLKTVXAQITQPRDNYSLNLASRLYAEESYPILPEYLQCVK |
| | | ELYNGGLETVSFQTAADQARELINSWVESQTNGIIKNILQPSSVDPQTEMVLVNAIYFKGLWEKAF |
| | | KDEETQAVPFRITEQENRPVQMMYQFGSFKVAXVASEKIKILELPYASGQLSMLVLLPDEVSGLEQ |
| | | NAITFEKLTEWTSSDLMEERKIKVFFPRVKIEEKYNLTAVLVSLGITDLFSSSANLSGISSAENLKMS |
| | | EAVHEAXVEIYEAGSEVAGSSGAGIEVASDSEEFRVDHPFLFLIXHNPTNSILFFGRCFSP |
| PREDICTED: | SEQ ID | MGSIGAASTEFCIDVFRELRVQHVNENIFYSPLSIISALSMVYLGARENTRAQIDEVFHFDKIAGFGD |
| Ovalbumin-like | NO: 97 | TVDPQCGASLSVHKSLQNVFAQITQPKDNYSLNLASRLYAEESYPILPEYLQCVKELYNEGLETVSF |
| [Melopsittacus | | QTGADQARELINSWVENQTNGVIKNILQPSSVDPQTEMVLVNAIYFKGLWQKAFKDEETQAVPFRI |
| undulatus] | | TEQENRPVQMMYQFGSFKVAVVASEKVKILELPYASGQLSMWVLLPDEVSGLEQLENAITFEKLT |
| | | EWTSSDLTEERKIKVFLPRVKIEEKYNLTAVLMALGVTDLFSSSANFSGISAAENLKMSEAVHEAF |
| | | VEIYEAGSEVVGSSGAGIEAPSDSEEFRADHPFLFLIKHNPTNSILFFGRCFSP |
| Ovalbumin-like | SEQ ID | MGSIGPLSVEFCCDVFKELRIQHARDNIFYSPVTIISALSMVYLGARDNTKAQIEKAVHFDKIPGFGE |
| [Neopelma | NO: 98 | SIESQCGTSLSVHTSLKDIFTQITKPRENYTVGIASRLYAEEKYPILPEYLQCIKELYKGGLEPISFQTA |
| chrysocephalum] | | AEQARELINSWVESQTNGMIKNILQPSSVNPETDMVLVNAIYFKGLWKKAFKDEGTQTVPFRITEQ |
| | | ESKPVQMMFQIGSFRVAEITSEKIRILELPYASGQLSLWVLLPDDISGLEQLESAITFENLKEWTSSTK |
| | | MEERKIKVYLPRMKIEEKYNLTSVLTSLGITDLFSSSANLSGISSAEKLKVSSAFHEASMEIYEAGNK |
| | | VVGSTGAGVDDTSVSEEFRVDRPFLFLIKHNPSNSIFFFGRCFSP |
| PREDICTED: | SEQ ID | MGSIGAASAEFCVDVFKELKDQHVNNIVFSPLMIISALSMVNIGAREDTRAQIDKVVHFDKITGYGE |
| Ovalbumin-like | NO: 99 | SIESQCGTSIGIYFSLKDAFTQITKPSDNYSLSFASKLYAEETYPILPEYLKCVKELYKGGLETISFQTA |
| [Buceros rhinoceros | | ADQARELINSWVESQTNGMIKNILQPSSVDPQTEMVLVNAIYFKGLWEKAFKDEDTQAVPFRITEQ |
| silvestris] | | ESKPVQMMYQIGSFKVAVIASEKIKILELPYASGQLSLLVLLPDDVSGLEQLESAITSEKLLEWTNPN |
| | | IMEERKTKVYLPRMKIEEKYNLTSVLVALGITDLFSSSANLSGISSAEGLKLSDAVHEAFVEIYEAG |
| | | REVVGSSEAGVEDSSVSEEFKADRPFIFLIKHNPTNGILYFGRYISP |
| PREDICTED: | SEQ ID | MGSIGAANTDFCFDVFKELKVHHANENIFYSPLSIVSALAMVYLGARENTRAQIDKALHFDKILGF |
| Ovalbumin-like | NO: 100 | GETVESQCDTSVSVHTSLKDMLIQITKPSDNYSFSFASKIYTEETYPILPEYLQCVKELYKGGVETISF |
| [Cariama cristata] | | QTAADQAREVINSWVESHTNGMIKNILQPGSVDPQTKMVLVNAVYFKGIWEKAFKEEDTQEMPF |
| | | RINEQESKPVQMMYQIGSFKLTVAASENLKILEFPYASGQLSMMVILPDEVSGLKQLETSITSEKLIK |
| | | WTSSNTMEERKIRVYLPRMKIEEKYNLKSVLMALGITDLFSSSANLSGISSAESLKMSEAVHEAFVE |
| | | IYEAGSEVTSSTGTEMEAENVSEEFKADHPFLFLIKHNPTDSIVFFGRCMSP |
| Ovalbumin [Manacus | SEQ ID | MGSIGPLSVEFCCDVFKELRIQHARENIFYSPVTIISALSMVYLGARDNTKAQIEKAVHFDKIPGFGE |
| vitellinus] | NO: 101 | SIESQCGTSLSIHTSLKDIFTQITKPSDNYTVGIASRLYAEEKYPILPEYLQCIKELYKGGLEPISFQTA |
| | | AEQARELINSWVESQTNGMIKNILQPSSVNPETDMVLVNAIYFKGLWEKAFKDESTQTVPFRITEQ |
| | | ESKPVQMMFQIGSFRVAEIASEKIRILELPYASGQLSLWVLLPDDISGLEQLETAITFENLKEWTSST |
| | | KMEERKIKVYLPRMKIEEKYNLTSVLTSLGITDLFSSSANLSGISSAERLKVSSAFHEASMEIYEAGS |
| | | RVVEAGVDDTSVSEEFRVDRPFLFLIKHNPSNSIFFFGRCFSP |
| Ovalbumin-like | SEQ ID | MGSIGPVSTEFCCDIFKELRIQHARENIIYSPVTIISALSMVYLGARDNTKAQIEKAVHFDKIPGFGESI |
| [Empidonax traillii] | NO: 102 | ESQCGTSLSIHTSLKDILTQITKPSDNYTVGIASRLYAEEKYPILSEYLQCIKELYKGGLEPISFQTAAE |
| | | QARELINSWVESQTNGMIKNILQPSSVNPETDMVLVNAIYFKGLWEKAFKDEGTQTVPFRITEQES |
| | | KPVQMMFQIGSFKVAEITSEKIRILELPYASGKLSLWVLLPDDISGLEQLETAITFENLKEWTSSTRM |
| | | EERKIKVYLPRMKIEEKYNLTSVLTSLGITDLFSSSANLSGISSAERLKVSSAFHEVFVEIYEAGSKVE |
| | | GSTGAGVDDTSVSEEFRADHPFLFLVKHNPSNSIIFFGRCYLP |
| PREDICTED: | SEQ ID | MGSTGAASMEFCFALFRELKVQHVNENIFFSPVTIISALSMVYLGARENTRAQLDKVAPFDKITGFG |
| Ovalbumin-like | NO: 103 | ETIGSQCSTSASSHTSLKDVFTQITKASDNYSLSFASRLYAEETYPILPEYLQCVKELYKGGLESISFQ |
| [Leptosomus discolor] | | TAADQARELINSWVESQTNGMIKDILRPSSVDPQTKIILITAIYFKGMWEKAFKEEDTQAVPFRMTE |
| | | QESKPVQMMYQIGSFKVAVIPSEKLKILELPYASGQLSMLVILPDDVSGLEQLETAITTEKLKEWTS |
| | | PSMMKERKMKVYFPRMRIEEKYNLTSVLMALGITDLFSPSANLSGISSAESLKVSEAVHEASVDIDE |
| | | AGSEVIGSTGVGTEVTSVSEEIRADHPFLFLIKHKPTNSILFFGRCFSP |
| Hypothetical protein | SEQ ID | MEHAQLTQLVNSNMTSNTCHEADEFENIDFRMDSISVTNTKFCFDVFNEMKVHHVNENILYSPLSI |
| H355_008077 [Colinus | NO: 104 | LTALAMVYLGARGNTESQMKKALHFDSITGAGSTTDSQCGSSEYIHNLFKEFLTEITRTNATYSLEI |
| virginianus] | | ADKLYVDKTFTVLPEYINCARKFYTGGVEEVNFKTAAEEARQLINSWVEKETNGQIKDLLVPSSVD |
| | | FGTMMVFINTIYFKGIWKTAFNTEDTREMPFSMTKQESKPVQMMCLNDTFNMATLPAEKMRILEL |
| | | PYASGELSMLVLLPDEVSGLEQIEKAINFEKLREWTSTNAMEKKSMKVYLPRMKIEEKYNLTSTLM |
| | | ALGMTDLFSRSANLTGISSVENLMISDAVHGAFMEVNEEGTEAAGSTGAIGNIKHSVEFEEFRADH |
| | | PFLFLIRYNPTNVILFFDNSEFTMGSIGAVSTEFCFDVFKELRVHHANENIFYSPFTVISALAMVYLG |
| | | AKDSTRTQINKVVRFDKLPGFGDSIEAQCGTSANVHSSLRDILNQITKPNDIYSFSLASRLYADETYT |
| | | ILPEYLQCVKELYRGGLESINFQTAADQARELINSWVESQTSGIIRNVLQPSSVDSQTAMVLVNAIY |
| | | FKGLWEKGFKDEDTQAMPFRVTEQENKSVQMMYQIGTFKVASVASEKMKILELPFASGTMSMW |
| | | VLLPDEVSGLEQLETTISIEKLTEWTSSSVMEERKIKVFLPRMKMEEKYNLTSVLMAMGMTDLFSS |
| | | SANLSGISSTLQKKGFRSQELGDKYAKPMLESPALTPQVTAWDNSWIVAHPAAIEPDLCYQIMEQK |
| | | WKPFDWPDFRLPMRVSCRFRTMEALNKANTSFALDFFKHECQEDDDENILFSPFSISSALATVYLG |
| | | AKGNTADQMAKTEIGKSGNIHAGFKALDLEINQPTKNYLLNSVNQLYGEKSLPFSKEYLQLAKKY |
| | | YSAEPQSVDFLGKANEIRREINSRVEHQTEGKIKNLLPPGSIDSLTRLVLVNALYFKGNWATKFEAE |
| | | DTRHRPFRINMHTTKQVPMMYLRDKFNWTYVESVQTDVLELPYVNNDLSMFILLPRDITGLQKLI |
| | | NELTFEKLSAWTSPELMEKMKMEVYLPRFTVEKKYDMKSTLSKMGIEDAFTKVDSCGVTNVDEIT |
| | | THIVSSKCLELKHIQINKKLKCNKAVAMEQVSASIGNFTIDLFNKLNETSRDKNIFFSPWSVSSALAL |
| | | TSLAAKGNTAREMAEDPENEQAENIHSGFKELMTALNKPRNTYSLKSANRIYVEKNYPLLPTYIQL |
| | | SKKYYKAEPYKVNFKTAPEQSRKEINNWVEKQTERKIKNFLSSDDVKNSTKSILVNAIYFKAEWEE |
| | | KFQAGNTDMQPFRMSKNKSKLVKMMYMRHTFPVLIMEKLNFKMIELPYVKRELSMFILLPDDIKD |
| | | STTGLEQLERELTYEKLSEWADSKKMSVTLVDLHLPKFSMEDRYDLKDALKSMGMASAFNSNAD |
| | | FSGMTGFQAVPMESLSASTNSFTLDLYKKLDETSKGQNIFFASWSIATALAMVHLGAKGDTATQV |
| | | AKGPEYEETENIHSGFKELLSAINKPRNTYLMKSANRLFGDKTYPLLPKFLELVARYYQAKPQAVN |
| | | FKTDAEQARAQINSWVENETESKIQNLLPAGSIDSHTVLVLVNAIYFKGNWEKRFLEKDTSKMPFR |
| | | LSKTETKPVQMMFLKDTFLIHHERTMKFKIIELPYVGNELSAFVLLPDDISDNTTGLELVERELTYE |
| | | KLAEWSNSASMMKAKVELYLPKLKMEENYDLKSVLSDMGIRSAFDPAQADFTRMSEKKDLFISK |
| | | VIHKAFVEVNEEDRIVQLASGRLTGRCRTLANKELSEKNRTKNLFFSPFSISSALSMILLGSKGNTEA |
| | | QIAKVLSLSKAEDAHNGYQSLLSEINNPDTKYILRTANRLYGEKTFEFLSSFIDSSQKFYHAGLEQT |
| | | DFKNASEDSRKQINGWVEEKTEGKIQKLLSEGIINSMTKLVLVNAIYFKGNWQEKFDKETTKEMPF |
| | | KINKNETKPVQMMFRKGKYNMTYIGDLETTVLEIPYVDNELSMIILLPDSIQDESTGLEKLERELTY |
| | | EKLMDWINPNMMDSTEVRVSLPRFKLEENYELKPTLSTMGMPDAFDLRTADFSGISSGNELVLSEV |
| | | VHKSFVEVNEEGTEAAAATAGIMLLRCAMIVANFTADHPFLFFIRHNKTNSILFCGRFCSP |
| PREDICTED: | SEQ ID | MGSIGTASTEFCFDMFKEMKVQHANQNIIFSPLTIISALSMVYLGARDNTKAQMEKVIHFDKITGFG |
| Ovalbumin isoform X2 | NO: 105 | ESVESQCGTSVSIHTSLKDMLSEITKPSDNYSLSLASRLYAEETYPILPEYLQCMKELYKGGLETVSF |
| [Apteryx australis | | QTAADQARELINSWVESQTNGVIKNFLQPGSVDPQTEMVLVNAIYFKGMWEKAFKDEDTQEVPFR |
| mantelli] | | ITEQESKPVQMMYQVGSFKVATVAAEKMKILEIPYTHRELSMFVLLPDDISGLEQLETTISFEKLTE |
| | | WTSSNMMEERKVKVYLPHMKIEEKYNLTSVLMALGMTDLFSPSANLSGISTAQTLMMSEAIHGA |
| | | YVEIYEAGREMASSTGVQVEVTSVLEEVRADKPFLFFIRHNPTNSMVVFGRYMSP |
| Hypothetical protein | SEQ ID | MTSNTCHEADEFENIDFRMDSISVTNTKFCFDVFNEMKVHHVNENILYSPLSILTALAMVYLGARG |
| ASZ78_006007 | NO: 106 | NTESQMKKALHFDSITGGGSTTDSQCGSSEYIHNLFKEFLTEITRTNATYSLEIADKLYVDKTFTVLP |
| [Callipepla squamata] | | EYINCARKFYTGGVEEVNFKTAAEEARQLMNSWVEKETNGQIKDLLVPSSVDFGTMMVFINTIYF |
| | | KGIWKTAFNTEDTREMPFSMTKQESKPVQMMCLNDTFNMVTLPAEKMRILELPYASGELSMLVLL |
| | | PDEVSGLERIEKAINFEKLREWTSTNAMEKKSMKVYLPRMKIEEKYNLTSTLMALGMTDLFSRSA |
| | | NLTGISSVDNLMISDAVHGAFMEVNEEGTEAAGSTGAIGNIKHSVEFEEFRADHPFLFLIRYNPTNVI |
| | | LFFDNSEFTMGSIGAVSTEFCFDVFKELRVHHANENIFYSPFTIISALAMVYLGAKDSTRTQINKVVR |
| | | FDKLPGFGDSIEAQCGTSANVHSSLRDILNQITKPNDIYSFSLASRLYADETYTILPEYLQCVKELYR |
| | | GGLESINFQTAADQARELINSWVESQTSGIIRNVLQPSSVDSQTAMVLVNAIYFKGLWEKGFKDED |
| | | TQAIPFRVTEQENKSVQMMYQIGTFKVASVASEKMKILELPFASGTMSMWVLLPDEVSGLEQLET |
| | | TISIEKLTEWTSSSVMEERKIKVFLPRMKMEEKYNLTSVLMAMGMTDLFSSSANLSGISSTLQKKGF |
| | | RSQELGDKYAKPMLESPALTPQATAWDNSWIVAHPPAIEPDLYYQIMEQKWKPFDWPDFRLPMR |
| | | VSCRFRTMEALNKANTSFALDFFKHECQEDDSENILFSPFSISSALATVYLGAKGNTADQMAKVLH |
| | | FNEAEGARNVTTTIRMQVYSRTDQQRLNRRACFQKTEIGKSGNIHAGFKGLNLEINQPTKNYLLNS |
| | | VNQLYGEKSLPFSKEYLQLAKKYYSAEPQSVDFVGTANEIRREINSRVEHQTEGKIKNLLPPGSIDS |
| | | LTRLVLVNALYFKGNWATKFEAEDTRHRPFRINTHTTKQVPMMYLSDKFNWTYVESVQTDVLEL |
| | | PYVNNDLSMFILLPRDITGLQKLINELTFEKLSAWTSPELMEKMKMEVYLPRFTVEKKYDMKSTLS |
| | | KMGIEDAFTKVDNCGVTNVDEITIHVVPSKCLELKHIQINKELKCNKAVAMEQVSASIGNFTIDLFN |
| | | KLNETSRDKNIFFSPWSVSSALALTSLAAKGNTAREMAEDPENEQAENIHSGFNELLTALNKPRNT |
| | | YSLKSANRIYVEKNYPLLPTYIQLSKKYYKAEPHKVNFKTAPEQSRKEINNWVEKQTERKIKNFLSS |
| | | DDVKNSTKLILVNAIYFKAEWEEKFQAGNTDMQPFRMSKNKSKLVKMMYMRHTFPVLIMEKLNF |
| | | KMIELPYVKRELSMFILLPDDIKDSTTGLEQLERELTYEKLSEWADSKKMSVTLVDLHLPKFSMED |
| | | RYDLKDALRSMGMASAFNSNADFSGMTGERDLVISKVCHQSFVAVDEKGTEAAAATAVIAEAVP |
| | | MESLSASTNSFTLDLYKKLDETSKGQNIFFASWSIATALTMVHLGAKGDTATQVAKGPEYEETENI |
| | | HSGFKELLSALNKPRNTYSMKSANRLFGDKTYPLLPTKTKPVQMMFLKDTFLIHHERTMKFKIIEL |
| | | PYMGNELSAFVLLPDDISDNTTGLELVERELTYEKLAEWSNSASMMKVKVELYLPKLKMEENYDL |
| | | KSALSDMGIRSAFDPAQADFTRMSEKKDLFISKVIHKAFVEVNEEDRIVQLASGRLTGNTEAQIAK |
| | | VLSLSKAEDAHNGYQSLLSEINNPDTKYILRTANRLYGEKTFEFLSSFIDSSQKFYHAGLEQTDFKN |
| | | ASEDSRKQINGWVEEKTEGKIQKLLSEGIINSMTKLVLVNAIYFKGNWQEKFDKETTKEMPFKINK |
| | | NETKPVQMMFRKGKYNMTYIGDLETTVLEIPYVDNELSMIILLPDSIQDESTGLEKLERELTYEKLM |
| | | DWINPNMMDSTEVRVSLPRFKLEENYELKPTLSTMGMPDAFDLRTADFSGISSGNELVLSEVVHKS |
| | | FVEVNEEGTEAAAATAGIMLLRCAMIVANFTADHPFLFFIRHNKTNSILFCGRFCSP |
| PREDICTED: | SEQ ID | MASIGAASTEFCFDVFKELKTQHVKENIFYSPMAIISALSMVYIGARENTRAEIDKVVHFDKITGFG |
| Ovalbumin-like | NO: 107 | NAVESQCGPSVSVHSSLKDLITQISKRSDNYSLSYASRIYAEETYPILPEYLQCVKEVYKGGLESISF |
| [Mesitornis unicolor] | | QTAADQARENINAWVESQTNGMIKNILQPSSVNPQTEMVLVNAIYLKGMWEKAFKDEDTQTMPF |
| | | RVTQQESKPVQMMYQIGSFKVAVIASEKMKILELPYTSGQLSMLVLLPDDVSGLEQVESAITAEKL |
| | | MEWTSPSIMEERTMKVYLPRMKMVEKYNLTSVLMALGMTDLFTSVANLSGISSAQGLKMSQAIH |
| | | EAFVEIYEAGSEAVGSTGVGMEITSVSEEFKADLSFLFLIRHNPTNSIIFFGRCISP |
| Ovalbumin, partial | SEQ ID | MGSIGAASTEFCFDVFRELRVQHVNENIFYSPFSIISALAMVYLGARDNTRTQIDKISQFQALSDEHL |
| [Anas platyrhynchos] | NO: 108 | VLCIQQLGEFFVCTNRERREVTRYSEQTEDKTQDQNTGQIHKIVDTCMLRQDILTQITKPSDNFSLS |
| | | FASRLYAEETYAILPEYLQCVKELYKGGLESISFQTAADQARELINSWVESQTNGIIKNILQPSSVDS |
| | | QTTMVLVNAIYFKGMWEKAFKDEDTQAMPFRMTEQESKPVQMMYQVGSFKVAMVTSEKMKILE |
| | | LPFASGMMSMFVLLPDEVSGLEQLESTISFEKLTEWTSSTMMEERRMKVYLPRMKMEEKYNLTSV |
| | | FMALGMTDLFSSSANMSGISSTVSLKMSEAVHAACVEIFEAGRDVVGSAEAGMDVTSVSEEFRAD |
| | | HPFLFFIKHNPTNSILFFGRWMSP |
| PREDICTED: | SEQ ID | MGSIGAASAEFCLDIFKELKVQHVNENIIFSPMTIISALSLVYLGAKEDTRAQIEKVVPFDKIPGFGEI |
| Ovalbumin-like | NO: 109 | VESQCPKSASVHSSIQDIFNQIIKRSDNYSLSLASRLYAEESYPIRPEYLQCVKELDKEGLETISFQTA |
| [Chaetura pelagica] | | ADQARQLINSWVESQTNGMIKNILQPSSVNSQTEMVLVNAIYFRGLWQKAFKDEDTQAVPFRITEQ |
| | | ESKPVQMMQQIGSFKVAEIASEKMKILELPYASGQLSMLVLLPDDVSGLEKLESSITVEKLIEWTSS |
| | | NLTEERNVKVYLPRLKIEEKYNLTSVLAALGITDLFSSSANLSGISTAESLKLSRAVHESFVEIQEAG |
| | | HEVEGPKEAGIEVTSALDEFRVDRPFLFVTKHNPTNSILFLGRCLSP |
| PREDICTED: | SEQ ID | MGSISAASGEFCLDIFKELKVQHVNENIFYSPMVIVSALSLVYLGARENTRAQIDKVIPFDKITGSSE |
| Ovalbumin-like | NO: 110 | AVESQCGTPVGAHISLKDVFAQIAKRSDNYSLSFVNRLYAEETYPILPEYLQCVKELYKGGLETISF |
| [Apaloderma vittatum] | | QTAADQAREIINSWVESQTDGKIKNILQPSSVDPQTKMVLVSAIYFKGLWEKSFKDEDTQAVPFRV |
| | | TEQESKPVQMMYQIGSFKVAAIAAEKIKILELPYASEQLSMLVLLPDDVSGLEQLEKKISYEKLTEW |
| | | TSSSVMEEKKIKVYLPRMKIEEKYNLTSILMSLGITDLFSSSANLSGISSTKSLKMSEAVHEASVEIYE |
| | | AGSEASGITGDGMEATSVFGEFKVDHPFLFMIKHKPTNSILFFGRCISP |
| Ovalbumin-like | SEQ ID | MGSIGPVSTEVCCDIFRELRSQSVQENVCYSPLLIISTLSMVYIGAKDNTKAQIEKAIHFDKIPGFGES |
| [Corvus cornix cornix] | NO: 111 | TESQCGTSVSIHTSLKDIFTQITKPSDNYSISIARRLYAEEKYPILPEYIQCVKELYKGGLESISFQTAA |
| | | EKSRELINSWVESQTNGTIKNILQPSSVSSQTDMVLVSAIYFKGLWEKAFKEEDTQTIPFRITEQESK |
| | | PVQMMSQIGTFKVAEIPSEKCRILELPYASGRLSLWVLLPDDISGLEQLETAITFENLKEWTSSSKME |
| | | ERKIRVYLPRMKIEEKYNLTSVLKSLGITDLFSSSANLSGISSAESLKVSAAFHEASVEIYEAGSKGV |
| | | GSSEAGVDGTSVSEEIRADHPFLFLIKHNPSDSILFFGRCFSP |
| PREDICTED: | SEQ ID | MGSIGAASTEFCFDVFKELKVQHVNENIIISPLSIISALSMVYLGAREDTRAQIDKVVHFDKITGFGE |
| Ovalbumin-like | NO: 112 | AIESQCPTSESVHASLKETFSQLTKPSDNYSLAFASRLYAEETYPILPEYLQCVKELYKGGLETINFQ |
| [Calypte anna] | | TAAEQARQVINSWVESQTDGMIKSLLQPSSVDPQTEMILVNAIYFRGLWERAFKDEDTQELPFRITE |
| | | QESKPVQMMSQIGSFKVAVVASEKVKILELPYASGQLSMLVLLPDDVSGLEQLESSITVEKLIEWIS |
| | | SNTKEERNIKVYLPRMKIEEKYNLTSVLVALGITDLFSSSANLSGISSAESLKISEAVHEAFVEIQEAG |
| | | SEVVGSPGPEVEVTSVSEEWKADRPFLFLIKHNPTNSILFFGRYISP |
| PREDICTED: | SEQ ID | MGSIGPVSTEVCCDIFRELRSQSVQENVCYSPLLIISTLSMVYIGAKDNTKAQIEKAIHFDKIPGFGES |
| Ovalbumin [Corvus | NO: 113 | TESQCGTSVSIHTSLKDIFTQITKPSDNYSISIARRLYAEEKYPILQEYIQCVKELYKGGLESISFQTAA |
| brachyrhynchos] | | EKSRELINSWVESQTNGTIKNILQPSSVSSQTDMVLVSAIYFKGLWEKAFKEEDTQTIPFRITEQESK |
| | | PVQMMSQIGTFKVAEIPSEKCRILELPYASGRLSLWVLLPDDISGLEQLETSITFENLKEWTSSSKME |
| | | ERKIRVYLPRMKIEEKYNLTSVLKSLGITDLFSSSANLSGISSAESLKVSAVFHEASVEIYEAGSKGV |
| | | GSSEAGVDGTSVSEEIRADHPFLFLIKHNPSDSILFFGRCFSP |
| Hypothetical protein | SEQ ID | MLNLMHPKQFCCTMGSIGPVSTEVCCDIFRELRSQSVQENVCYSPLLIISTLSMVYIGAKDNTKAQI |
| DUI87_08270 | NO: 114 | EKAIHFDKIPGFGESTESQCGTSVSIHTSLKDIFTQITKPSDNYSISIASRLYAEEKYPILPEYIQCVK |
| [Hirundorustica | | ELYKGGLESISFQTAAEKSRELINSWVESQTNGTIKNILQPSSVSSQTDMVLVSAIYFKGLWEKAFKEE |
| rustica] | | DTQTVPFRITEQESKPVQMMSQIGTFKVAEIPSEKCRILELPYASGRLSLWVLLPDDISGLEQLETAIT |
| | | SENLKEWTSSSKMEERKIKVYLPRMKIEEKYNLTSVLKSLGITDLFSSSANLSGISSAESLKVSGAFH |
| | | EAFVEIYEAGSKAVGSSGAGVEDTSVSEEIRADHPFLFFIKHNPSDSILFFGRCFSP |
| Ostrich OVA sequence | SEQ ID | EAEAGSIGTASAEFCFDVFKELKVHHVNENIFYSPLSIISALSMVYLGARENTKTQMEKVIHFDKITG |
| as secreted from pichia | NO: 115 | LGESMESQCGTGVSIHTALKDMLSEITKPSDNYSLSLASRLYAEQTYAILPEYLQCIKELYKESLETV |
| | | SFQTAADQARELINSWIESQTNGVIKNFLQPGSVDSQTELVLVNAIYFKGMWEKAFKDEDTQEVPF |
| | | RITEQESRPVQMMYQAGSFKVATVAAEKIKILELPYASGELSMLVLLPDDISGLEQLETTISFEKLTE |
| | | WTSSNMMEDRNMKVYLPRMKIEEKYNLTSVLIALGMTDLFSPAANLSGISAAESLKMSEAIHAAY |
| | | VEIYEADSEIVSSAGVQVEVTSDSEEFRVDHPFLFLIKHNPTNSVLFFGRCISP |
| Ostrich construct | SEQ ID | MRFPSIFTAVLFAASSALAAPVNTTTEDETAQIPAEAVIGYSDLEGDFDVAVLPFSNSTNNGLLFINT |
| (secretion signal + | NO: 116 | TIASIAAKEEGVSLEKREAEAGSIGTASAEFCFDVFKELKVHHVNENIFYSPLSIISALSMVYLGARE |
| mature protein) | | NTKTQMEKVIHFDKITGLGESMESQCGTGVSIHTALKDMLSEITKPSDNYSLSLASRLYAEQTYAIL |
| | | PEYLQCIKELYKESLETVSFQTAADQARELINSWIESQTNGVIKNFLQPGSVDSQTELVLVNAIYFK |
| | | GMWEKAFKDEDTQEVPFRITEQESRPVQMMYQAGSFKVATVAAEKIKILELPYASGELSMLVLLP |
| | | DDISGLEQLETTISFEKLTEWTSSNMMEDRNMKVYLPRMKIEEKYNLTSVLIALGMTDLFSPAANL |
| | | SGISAAESLKMSEAIHAAYVEIYEADSEIVSSAGVQVEVTSDSEEFRVDHPFLFLIKHNPTNSVLFFG |
| | | RCISP |
| Duck OVA sequence as | SEQ ID | EAEAGSIGAASTEFCFDVFRELRVQHVNENIFYSPFSIISALAMVYLGARDNTRTQIDKVVHFDKLP |
| secreted from pichia | NO: 117 | GFGESMEAQCGTSVSVHSSLRDILTQITKPSDNFSLSFASRLYAEETYAILPEYLQCVKELYKGGLES |
| | | ISFQTAADQARELINSWVESQTNGIIKNILQPSSVDSQTTMVLVNAIYFKGMWEKAFKDEDTQAMP |
| | | FRMTEQESKPVQMMYQVGSFKVAMVTSEKMKILELPFASGMMSMFVLLPDEVSGLEQLESTISFE |
| | | KLTEWTSSTMMEERRMKVYLPRMKMEEKYNLTSVFMALGMTDLFSSSANMSGISSTVSLKMSEA |
| | | VHAACVEIFEAGRDVVGSAEAGMDVTSVSEEFRADHPFLFFIKHNPTNSILFFGRWMSP |
| Duck construct | SEQ ID | MRFPSIFTAVLFAASSALAAPVNTTTEDETAQIPAEAVIGYSDLEGDFDVAVLPFSNSTNNGLLFINT |
| (secretion signal + | NO: 118 | TIASIAAKEEGVSLEKREAEAGSIGAASTEFCFDVFRELRVQHVNENIFYSPFSIISALAMVYLGARD |
| mature protein) | | NTRTQIDKVVHFDKLPGFGESMEAQCGTSVSVHSSLRDILTQITKPSDNFSLSFASRLYAEETYAILP |
| | | EYLQCVKELYKGGLESISFQTAADQARELINSWVESQTNGIIKNILQPSSVDSQTTMVLVNAIYFKG |
| | | MWEKAFKDEDTQAMPFRMTEQESKPVQMMYQVGSFKVAMVTSEKMKILELPFASGMMSMFVLL |
| | | PDEVSGLEQLESTISFEKLTEWTSSTMMEERRMKVYLPRMKMEEKYNLTSVFMALGMTDLFSSSA |
| | | NMSGISSTVSLKMSEAVHAACVEIFEAGRDVVGSAEAGMDVTSVSEEFRADHPFLFFIKHNPTNSIL |
| | | FFGRWMSP |
| rOVL as expressed in | SEQ ID | MRFPSIFTAVLFAASSALAAPVNTTTEDETAQIPAEAVIGYSDLEGDFDVAVLPFSNSTNNGLLFINT |
| pichia | NO: 119 | TIASIAAKEEGVSLDKREAEAKVFGRCELAAAMKRHGLDNYRGYSLGNWVCAAKFESNFNTQAT |
| bolded is an alpha | | NRNTDGSTDYGILQINSRWWCNDGRTPGSRNLCNIPCSALLSSDITASVNCAKKIVSDGNGMNAW |
| mating factor signal | | VAWRNRCKGTDVQAWIRGCRL |
| sequence | | |
| rOVL as found after | SEQ ID | EAEAKVFGRCELAAAMKRHGLDNYRGYSLGNWVCAAKFESNFNTQATNRNTDGSTDYGILQINS |
| secretion from Pichia | NO: 120 | RWWCNDGRTPGSRNLCNIPCSALLSSDITASVNCAKKIVSDGNGMNAWVAWRNRCKGTDVQAW |
| | | IRGCRL |
| Lysozyme (OVL) from | SEQ ID | KVFGRCELAAAMKRHGLDNYRGYSLGNWVCAAKFESNFNTQATNRNTDGSTDYGILQINSRWW |
| Gallus gallus (without | NO: 121 | CNDGRTPGSRNLCNIPCSALLSSDITASVNCAKKIVSDGNGMNAWVAWRNRCKGTDVQAWIRGC |
| signal sequence) | | RL |
| Lysozyme | SEQ ID | KVFGRCELAAAMKRHGLDNYRGYSLGNWVCVAKFESNFNTQATNRNTDGSTDYGILQINSRWW |
| | NO: 122 | CNDGRTPGSRNLCNIPCSALLSSDITASVNCAKKIVSDGNGMSAWVAWRNRCKGTDVQAWIRGC |
| | | RL |
| Lysozyme C (Human) | SEQ ID | KVFERCELARTLKRLGMDGYRGISLANWMCLAKWESGYNTRATNYNAGDRSTDYGIFQINSRYW |
| | NO: 123 | CNDGKTPGAVNACHLSCSALLQDNIADAVACAKRVVRDPQGIRAWVAWRNRCQNRDVRQYVQ |
| | | GCGV |
| Lysozyme C (Bos | SEQ ID | KVFERCELARTLKKLGLDGYKGVSLANWLCLTKWESSYNTKATNYNPSSESTDYGIFQINSKWWC |
| taurus) | NO: 124 | NDGKTPNAVDGCHVSCRELMENDIAKAVACAKHIVSEQGITAWVAWKSHCRDHDVSSYVEGCTL |
| Lysozyme (OVL) from | SEQ ID | MRSLLILVLCFLPLAALGKVFGRCELAAAMKRHGLDNYRGYSLGNWVCAAKFESNFNTQATNRN |
| Gallus gallus Native | NO: 125 | TDGSTDYGILQINSRWWCNDGRTPGSRNLCNIPCSALLSSDITASVNCAKKIVSDGNGMNAWVAW |
| secretion signal is | | RNRCKGTDVQAWIRGCRL |
| bolded | | |
| FPE1 | SEQ ID | RTDCYGNVNRIDTTGASCKTAKPEGLSYCGVSASKKIAERDLQAMDRYKTIIKKVGEKLCVEPAVI |
| | NO: 126 | AGIISRESHAGKVLKNGWGDRGNGFGLMQVDKRSHKPQGTWNGEVHITQGTTILINFIKTIQKKFP |
| | | SWTKDQQLKGGISAYNAGAGNVRSYARMDIGTTHDDYANDVVARAQYYKQHGY* |
| Axolotl (g) | SEQ ID | SGCYGNIMDVPTTGASCLTASQDNLPYCGVAASQQMAATDLPDMNQYKEKILAVAQNLCMDGA |
| (Ambystoma | NO: 127 | VIAGIISRESRAGAVLQNGWGDNGHAFGLMQIDIRWHSIEGAWNSQENINEGTGILINMIVAISDKF |
| mexicanum) | | PSWSVNDNLKGGIAAYNAGPGNIYSYSQVDQYTTDGDYSNDVVARAQYYKTQGY* |
| Pompano (g) | SEQ ID | FRYAILAREEEPRVRRAALVDKPRVEIADVLISTFTESGVIEVVLQALREIGCNDLRERFAKDTSEGS |
| (Trachinotus ovatus) | NO: 128 | PTSASKYGDIMKVETTGASMQTAQQDYLDFSGARASHAMAETDLIEMNNYKSVIKNAAGKKGVD |
| | | PALIAAMISRSCRAGKTLSGGWGCWDEKRQKYNTYGLMQIDVNPKGGGHTPKGSWDSEEHLCQA |
| | | IDILIRFITRIRQKYPQWSKEEQLKGGIAAYNAGDGNIGPGKDVDSKTTNGDYANDIVARAQWYKS |
| | | NGGF* |
| Chlamysin (i)(Chlamys | SEQ ID | AHNFATGIVPQSCLECICKTESGCRAIGCKFDVYSDSCGYFQLKQAYWEDCGRPGGSLTSCADDIH |
| islandica) | NO: 129 | CSSQCVQHYMSRYIGHTSCSRTCESYARLHNGGPHGCEHGSTLGYWGHVQGHGC* |

A recombinant protein such as rOVD, rOVA or rOVL can include additional sequences. Expression of recombinant proteins in a host cell, for instance a Pichia species, a Saccharomyces species, a Trichoderma species, a Pseudomonas species may lead to an addition of peptides to the protein sequence as part of post-transcriptional or post-translational modifications. Such peptides may not be part of the native protein sequences. For instance, expressing an OVD sequence in a Pichia species, such as Komagataella phaffii and Komagataella pastoris may lead to addition of a peptide at the N-terminus or C-terminus. In some cases, a tetrapeptide EAEA (SEQ ID NO: 130) is added to the N-terminus of the OVD sequence upon expression in a host cell. In some embodiments, rOVD or rOVA or both include the amino acids EAEA at the N-terminus. A recombinant protein sequence can include a signal sequence, such as for directing secretion from a host cell. In some cases, the signal sequence may be a native signal sequence. In some cases, a signal sequence may be a heterologous signal sequence. For instance, an alpha mating factor signal sequence can be fused to a sequence for expression and secretion in a yeast cell such as a Pichia sp. In some cases, the signal sequence is removed in whole or in part when the protein, such as an rOVD, rOVL or rOVA, is secreted from the host cell.

A recombinant protein such as rOVD, rOVA and/or rOVL can be a non-naturally occurring variant of an OVD, OVA and/or OVL. Such variant can comprise one or more amino acid insertions, deletions, or substitutions relative to a native OVD, native OVL or native OVA sequence.

Such an rOVD variant can have at least 70%, 75%, 80%, 85%, 90%, 95%, 96%, 97%, 98%, or 99% sequence identity to SEQ ID NOs: 1-44. An rOVA variant can have at least 70%, 75%, 80%, 85%, 90%, 95%, 96%, 97%, 98%, or 99% sequence identity to SEQ ID NOs: 45-118. An rOVL variant can have at least 70%, 75%, 80%, 85%, 90%, 95%, 96%, 97%, 98%, or 99% sequence identity to SEQ ID NOs: 119-129. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

In some embodiments, a variant is one that confers additional features, such as reduced allergenicity. For example, an rOVD can include G162M and/or F167A (such as in SEQ ID NO: 3) relative to a wild type OVD sequence SEQ ID NO: 2 and have reduced allergenicity as compared to the wild type OVD sequence.

Depending on the host organism used to express the recombinant proteins, such as rOVD, rOVL and/or rOVL can have a glycosylation, acetylation, or phosphorylation pattern different from wild-type OVD (e.g., native OVD) or wild-type OVA (e.g., native OVA). For example, the recombinant proteins herein may or may not be glycosylated, acetylated, or phosphorylated. A recombinant protein may have an avian, non-avian, microbial, non-microbial, mammalian, or non-mammalian glycosylation, acetylation, or phosphorylation pattern.

In some cases, recombinant proteins may be deglycosylated or modified in its glycosylation (e.g., chemically, enzymatically through endoglucanases (such as EndoH), endoglycosidases, mannosidases (such as alpha-1,2 mannosidase), PNGase F, O-Glycosidase, OCH1, Neuraminidase, β, 1-4 Galactosidase, β-N-acetylglucosaminidases, etc.), deacetylated (e.g., protein deacetylase, histone deacetylase, sirtuin), or dephosphorylated (e.g., acid phosphatase, lambda protein phosphatase, calf intestinal phosphatase, alkaline phosphatase). Deglycosylation, deacetylation or dephosphorylation may produce a protein that is more uniform or is capable of producing a composition with less variation.

The present disclosure contemplates modifying glycosylation of the rOVD to alter or enhance one or more functional characteristics of the protein and/or its production. A host cell may comprise heterologous enzymes that modify the glycosylation pattern of ovomucoid. In some cases, one or more enzymes may be used for modifying the glycosylation of rOVD protein. The enzymes used modifying glycosylation of rOVD may be an enzyme or a fusion protein comprising an enzyme or active fragment of an enzyme, for example EndoH or a fusion of OCH1 to EndoH (such as to provide for Golgi retention of the EndoH enzyme) may be provided in a host cell.

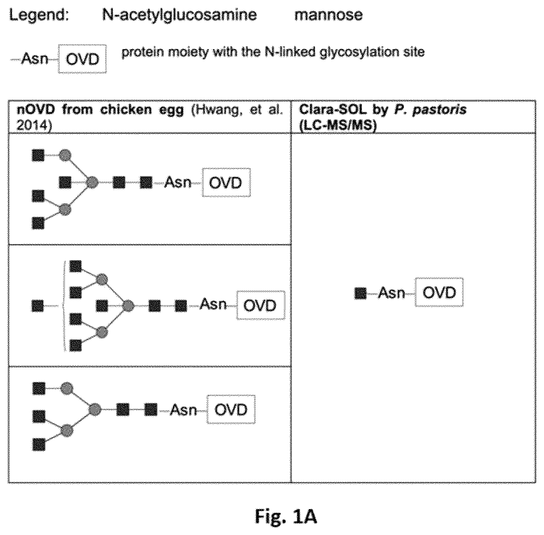

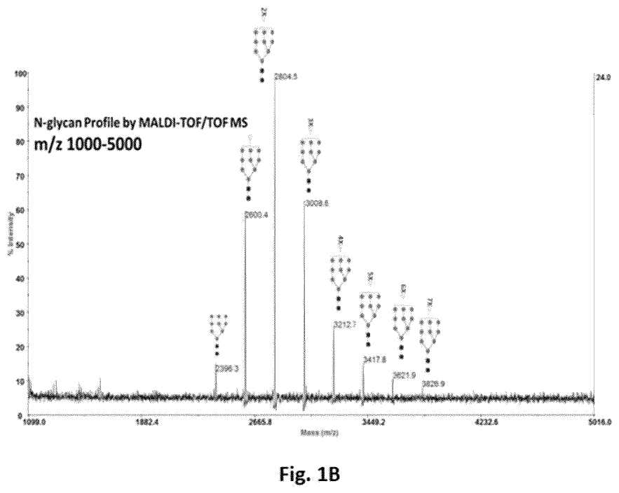

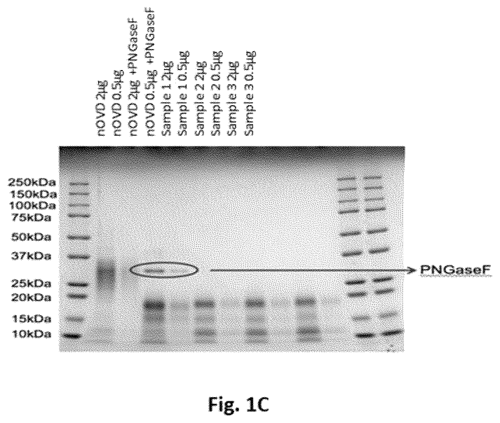

Native ovomucoid (nOVD), such as isolated from a chicken or other avian egg, has a highly complex branched form of glycosylation. The glycosylation pattern comprises N-linked glycan structures such as N-acetylglucosamine units and N-linked mannose units. See, e.g., FIG. 1B (left-hand column). In some cases, the rOVD for use in a herein-disclosed consumable composition and produced using the methods described herein has a glycosylation pattern which is different than the glycosylation pattern of nOVD. For example, when rOVD is produced in a Pichia sp., the protein may be highly glycosylated. FIG. 1C illustrates the glycosylation patterns of rOVD produced by P. pastoris, showing a complex branched glycosylation pattern. In some embodiments of the compositions and methods herein, rOVD is treated such that the glycosylation pattern is modified from that of nOVD and also modified as compared to rOVD produced by a Pichia sp. without such treatment. In some cases, the rOVD has no glycosylation. In other cases, the rOVD has reduced glycosylation. In some cases, the rOVD is modified by N-acetylglucosamine at one or more asparagine residues of the protein and lacks or is substantially devoid of N-linked mannosylation. See, e.g., FIG. 1B (right hand column). The changes in glycosylation described herein may lead to an increase in the solubility and clarity of rOVD as compared to other forms of protein such as whey proteins, soy proteins, pea proteins, and nOVD.

In some cases, an enzyme used for modifying glycosylation may be transformed into a host cell. In some cases, the enzyme used for modifying glycosylation may be transformed into the same host cell that produces rOVD. In some cases, the enzyme may be provided transiently to the host cell, such as by an inducible expression system. In some cases, when a host cell expresses an enzyme used for modifying glycosylation, the recombinant protein (e.g., rOVD and rOVA) is secreted from the host cell in the modified state.

In one example, a host cell producing OVD comprises a fusion of EndoH and OCH1 enzymes. An exemplary OCH1-EndoH protein sequence is provided as SEQ ID No: 119. In such cases, an rOVD produced from the host cell comprises a glycosylation pattern substantially different from an rOVD which is produced in a cell without such enzymes. The rOVD produced in such cases is also substantially different as compared to a native OVD (e.g., produced by a chicken or other avian egg). FIG. 1B shows a comparison of nOVD (with mannose residues) and rOVD glycosylation patterns wherein the rOVD was treated with EndoH and comprises an N-acetylglucosamine residue at the asparagine but no mannose residues. FIG. 1C shows the glycosylation pattern of rOVD produced in a host cell such as P. pastoris and where rOVD was not treated with EndoH and has both N-acetylglucosamine resides as well as the chains of N-linked mannose residues. Modification of the glycosylation of rOVD may provide nutritional benefits to rOVD, such as a higher nitrogen to carbon ratio, and may improve the clarity and solubility of the protein. In some cases, the modification of the glycosylation of rOVD is performed within the host cell that produces rOVD before the rOVD is secreted from the host cell and/or before isolating the rOVD. In some cases, modification of the glycosylation of rOVD is performed after its secretion and/or after isolating rOVD from the host cell.

The molecular weight or rOVD may be different as compared to nOVD. The molecular weight of the protein may be less than the molecular weight of nOVD or less than rOVD produced by the host cell where the glycosylation of rOVD is not modified. In embodiments, the molecular weight of an rOVD may be between 20 kDa and 40 kDa. In some cases, an rOVD with modified glycosylation has a different molecular weight, such as compared to a native OVD (as produced by an avian host species) or as compared to a host cell that glycosylates the rOVD, such as where the rOVD includes N-linked mannosylation. In some cases, the molecular weight of rOVD is greater than the molecular weight of the rOVD that is completely devoid of post-translational modifications or an rOVD that lacks all forms of N-linked glycosylation.

The present disclosure contemplates modifying glycosylation of the rOVA to alter or enhance one or more functional characteristics of the protein and/or its production. In some embodiments, the change in rOVA glycosylation can be due to the host cell glycosylating the rOVA. In some embodiments, rOVA has a glycosylation pattern that is not identical to a native ovalbumin (nOVA), such as a nOVA from chicken egg. In some embodiments, rOVA is treated with a deglycosylating enzyme before it is used as an ingredient in an rOVA composition, or when rOVA is present in a composition. In some embodiments, the glycosylation of rOVA is modified or removed by expressing one or more enzymes in a host cell and exposing rOVA to the one or more enzymes. In some embodiments, rOVA and the one or more enzymes for modification or removal of glycosylation are co-expressed in the same host cell.

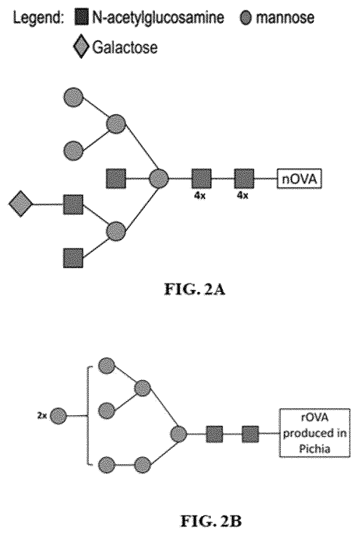

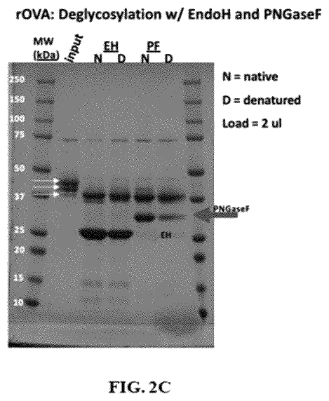

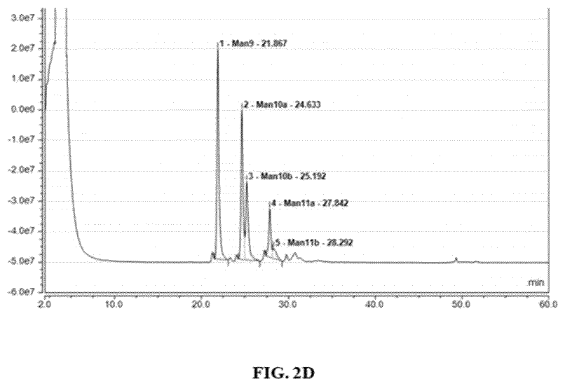

Native ovalbumin (nOVA), such as isolated from a chicken or another avian egg, has a highly complex branched form of glycosylation. The glycosylation pattern comprises N-linked glycan structures such as N-acetylglucosamine units, galactose and N-linked mannose units. See, e.g., FIG. 2A. In some cases, the rOVA for use in a herein disclosed consumable composition and produced using the methods described herein has a glycosylation pattern which is different from the glycosylation pattern of nOVA. For example, when rOVA is produced in a Pichia sp., the protein may be glycosylated differently from the nOVA and lack galactose units in the N-linked glycosylation. FIG. 2B illustrates the glycosylation patterns of rOVA produced by P. pastoris, showing a complex branched glycosylation pattern. In some embodiments of the compositions and methods disclosed herein, rOVA is treated such that the glycosylation pattern is modified from that of nOVA and also modified as compared to rOVA produced by a Pichia sp. without such treatment. In some cases, the rOVA lacks glycosylation.

The molecular weight or rOVA may be different as compared to nOVA. The molecular weight of the protein may be less than the molecular weight of nOVA or less than rOVA produced by the host cell where the glycosylation of rOVA is not modified. In embodiments, the molecular weight of an rOVA may be between 40 kDa and 55 kDa. In some cases, an rOVA with modified glycosylation has a different molecular weight, such as compared to a native OVA (as produced by an avian host species) or as compared to a host cell that glycosylates the rOVA, such as where the rOVA includes N-linked mannosylation. In some cases, the molecular weight of rOVA is greater than the molecular weight of the rOVA that is completely devoid of post-translational modifications. or an rOVA that lacks all forms of N-linked glycosylation.

Expression of an rOVD or rOVA can be provided by an expression vector, a plasmid, a nucleic acid integrated into the host genome or other means. For example, a vector for expression can include: (a) a promoter element, (b) a signal peptide, (c) a heterologous OVD or OVA sequence, and (d) a terminator element.

Expression vectors that can be used for expression of rOVD and rOVA include those containing an expression cassette with elements (a), (b), (c) and (d). In some embodiments, the signal peptide (c) need not be included in the vector. In general, the expression cassette is designed to mediate the transcription of the transgene when integrated into the genome of a cognate host microorganism.

To aid in the amplification of the vector prior to transformation into the host microorganism, a replication origin (e) may be contained in the vector (such as PUC_ORIC and PUC (DNA2.0)). To aide in the selection of microorganism stably transformed with the expression vector, the vector may also include a selection marker (f) such as URA3 gene and Zeocin resistance gene (ZeoR). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector element known to one of skill in the art can be used in combination or substituted for the elements described herein.

Exemplary promoter elements (a) may include, but are not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, a sequence or subsequence chosen from SEQ ID Nos: 121 to 132, and any combination thereof. Illustrative inducible promoters include methanol-induced promoters, e.g., DAS1 and pPEX11.

A signal peptide (b), also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides can be derived from a precursor of a protein other than the signal peptides in native recombinant proteins.

Any nucleic acid sequence that encodes recombinant proteins can be used as (c). Preferably such sequence is codon optimized for the species/genus/kingdom of the host cell.

Exemplary transcriptional terminator elements include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof.

Exemplary selectable markers (f) may include but are not limited to: an antibiotic resistance gene (e.g. zeocin, ampicillin, blasticidin, kanamycin, nurseothricin, chloroamphenicol, tetracycline, triclosan, ganciclovir, and any combination thereof), an auxotrophic marker (e.g. ade1, arg4, his4, ura3, met2, and any combination thereof).

In one example, a vector for expression in Pichia sp. can include an AOX1 promoter operably linked to a signal peptide (alpha mating factor) that is fused in frame with a nucleic acid sequence encoding recombinant proteins, and a terminator element (AOX1 terminator) immediately downstream of the nucleic acid sequence encoding the recombinant proteins.

In another example, a vector comprising a DAS1 promoter is operably linked to a signal peptide (alpha mating factor) that is fused in frame with a nucleic acid sequence encoding recombinant proteins and a terminator element (AOX1 terminator) immediately downstream of recombinant proteins.

A recombinant protein described herein may be secreted from the one or more host cells. In some embodiments, a recombinant protein is secreted from the host cell. The secreted recombinant protein may be isolated and purified by methods such as centrifugation, fractionation, filtration, affinity purification and other methods for separating protein from cells, liquid and solid media components and other cellular products and byproducts. In some embodiments, recombinant protein is produced in a Pichia Sp. and secreted from the host cells into the culture media. The secreted rO recombinant protein is then separated from other media components for further use.

In some cases, multiple vectors comprising recombinant proteins may be transfected into one or more host cells. A host cell may comprise more than one copy of recombinant proteins. A single host cell may comprise 2, 3, 4, 5, 6, 7, 8, 9, 10, 11, 12, 13, 14, 15, 16, 17, 18, 19 or 20 copies of recombinant proteins. A single host cell may comprise one or more vectors for the expression of recombinant proteins. A single host cell may comprise 2, 3, 4, 5, 6, 7, 8, 9 or 10 vectors for recombinant proteins expression. Each vector in the host cell may drive the expression of recombinant proteins using the same promoter. Alternatively, different promoters may be used in different vectors for recombinant proteins expression.

A recombinant protein such as rOVD, rOVA and/or rOVL is recombinantly expressed in one or more host cells. As used herein, a “host” or “host cell” denotes here any protein production host selected or genetically modified to produce a desired product. Exemplary hosts include fungi, such as filamentous fungi, as well as bacteria, yeast, plant, insect, and mammalian cells. A host cell may be Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, or Trichoderma vireus. A host cell can be an organism that is approved as generally regarded as safe by the U.S. Food and Drug Administration.

A recombinant protein can be recombinantly expressed in yeast, filamentous fungi or a bacterium. In some embodiments, recombinant protein is recombinantly expressed in a Pichia species (Komagataella phaffii and Komagataella pastoris), a Saccharomyces species, a Trichoderma species, a Trichoderma species, a Pseudomonas species or an E. coli species.

A host cell may be transformed to include one or more expression cassettes. As examples, a host cell may be transformed to express one expression cassette, two expression cassettes, three expression cassettes or more expression cassettes. In one example, a host cell is transformed express a first expression cassette that encodes rOVA and express a second expression cassette that encodes rOVD. In another example, a first host cell is transformed to express a first expression cassette that encodes rOVA and a second host cell is transformed to express a second expression cassette that encodes rOVD.

The consumable products and recombinant protein compositions herein can be essentially free of any microbial cells or microbial cell contaminants.

### Treated Proteins

The recombinant proteins such as rOVD, included in a recombinant protein containing composition, may be treated chemically or enzymatically before it is purified for use in a consumable composition or protein mixture. Such treatments may be performed to reduce impurities in a protein composition. Such treatments may be performed to improve the sensory attributes of the protein composition. Treatments may include but are not limited to purification steps, filtration, chemical treatments, and enzymatic treatments.

In some cases, rOVD protein and compositions containing rOVD protein, including forms of rOVD with modified glycosylation (e.g., such forms with N-acetylglucosamine but lacking N-linked mannose residues) may be treated with oxidizing agent or an oxygen-generating agent to modify components of the rOVD composition, such as impurities. The oxidizing agent or oxygen-generating agent may comprise hydrogen peroxide, sodium percarbonate, activated chlorine dioxide, bubbled oxygen or ozone. The treatment may improve the solubility and clarity of an rOVD composition. The treatment may reduce the odor of an rOVD composition. The treatment may neutralize the color of an rOVD composition; for instance, the rOVD composition may lose color after a treatment, e.g., to a less intense/lighter coloration. In embodiments, the color may change form greenish to yellowish and/or from yellowish to essentially colorless.

In some examples, rOVD may be treated with an oxidizing agent or an oxygen-generating agent, e.g., hydrogen peroxide or sodium percarbonate, before it is purified for use in a consumable composition. A culture medium comprising secreted or isolated rOVD may be treated with an oxygen-generating agent, e.g., hydrogen peroxide or sodium percarbonate. Using hydrogen peroxide as an example, a hydrogen peroxide treatment may be followed by one or more wash steps and/or filtration steps to remove hydrogen peroxide from the resulting rOVD compositions. Such steps may be performed following treatments with other oxygen-generating agents, e.g., sodium percarbonate.

In some cases, the concentration of hydrogen peroxide used for treating rOVD may be from 1% to 20%. The concentration of hydrogen peroxide used for treating rOVD may be at least 1% weight per total weight (w/w) and/or weight per total volume (w/v). The concentration of hydrogen peroxide used for treating rOVD may be at most 20% w/w or w/v. The concentration of hydrogen peroxide used for treating rOVD may be 1% to 2%, 1% to 5%, 1% to 7%, 1% to 10%, 1% to 12%, 1% to 15%, 1% to 17%, 1% to 20%, 2% to 5%, 2% to 7%, 2% to 10%, 2% to 12%, 2% to 15%, 2% to 17%, 2% to 20%, 5% to 7%, 5% to 10%, 5% to 12%, 5% to 15%, 5% to 17%, 5% to 20%, 7% to 10%, 7% to 12%, 7% to 15%, 7% to 17%, 7% to 20%, 10% to 12%, 10% to 15%, 10% to 17%, 10% to 20%, 12% to 15%, 12% to 17%, 12% to 20%, 15% to 17%, 15% to 20%, or 17% to 20% w/w or w/v. The concentration of hydrogen peroxide used for treating rOVD may be about 1%, 2%, 5%, 7%, 10%, 12%, 15%, 17%, or 20% w/w or w/v. The concentration of hydrogen peroxide used for treating rOVD may be at least 1%, 2%, 5%, 7%, 10%, 12%, 15% or 17% w/w or w/v. The concentration of hydrogen peroxide used for treating rOVD may be at most 2%, 5%, 7%, 10%, 12%, 15%, 17%, or 20% w/w or w/v.

rOVD may be treated with hydrogen peroxide for a limited duration of time. For instance, rOVD may be exposed to hydrogen peroxide for at least 1 hour, 2 hours, 3 hours, 5 hours, 7 hours, 10 hours, 12 hours, 15 hours, 17 hours, 20 hours, 22 hours, 24 hours, 26 hours, 28 hours, 30 hours, 34 hours, 36 hours, 40 hours, 44 hours or 48 hours. Hydrogen peroxide may be added to the rOVD culture media throughout the culturing process.

rOVD may be treated with hydrogen peroxide at a pH of about 3 to 6. rOVD may be treated with hydrogen peroxide at a pH of about 3, 3.2, 3.4, 3.6, 3.8, 4, 4.1, 4.2, 4.4, 4.6, 4.8, 5, 5.2, 5.4, 5.6, 5.8 or 6. rOVD may treated with hydrogen peroxide at a pH of at least 3, 3.2, 3.4, 3.6, 3.8, 4, 4.1, 4.2, 4.4, 4.6, 4.8, 5, 5.2, 5.4, 5.6 or 5.8. rOVD may treated with hydrogen peroxide at a pH of at most 3.2, 3.4, 3.6, 3.8, 4, 4.1, 4.2, 4.4, 4.6, 4.8, 5, 5.2, 5.4, 5.6, 5.8 or 6.

rOVD may be filtered before treatment with an oxygen-generating agent. In some cases, rOVD may be filtered before and after treatment with an oxygen-generating agent.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, weight per weight basis and weight per total weight basis are used synonymously. As used herein, weight per volume basis and weight per total volume basis are used synonymously.

As used herein, the singular forms “a”, “an,” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” As such, this statement is intended to serve as antecedent basis for use of such exclusive terminology as “solely,” “only,” and the like in connection with the recitation of claim elements or use of a “negative” limitation.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of” The phrase “consisting essentially of” is used herein to require the specified feature(s) as well as those which do not materially affect the character or function of the claimed disclosure. As used herein, the term “consisting” is used to indicate the presence of the recited feature alone.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. In another example, “about” can mean 10% greater than or less than the stated value. Where particular values are described in the application and claims, unless otherwise stated the term “about” should be assumed to mean an acceptable error range for the particular value. In some instances, the term “about” also includes the particular value. For example, “about 5” includes 5.

As used herein, “egg-less” refers to a product not containing any animal eggs (i.e., any natural egg white, natural whole egg, or natural egg yolk from a hen, ostrich, quail, duck, goose, turkey, pheasant, or other animal).

The terms “egg white,” “a whole egg,” or a “comparable composition without the fiber-providing component” includes the egg of a chicken, ostrich, quail, duck, goose, turkey, pheasant, or other animal. The comparable natural egg white, whole egg, or comparable composition without the fiber-providing component may be natural or unnatural (e.g., obtained from a genetically modified animal).

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

Any aspect or embodiment described herein can be combined with any other aspect or embodiment as disclosed herein.

Embodiment 1: A liquid whole egg substitute composition comprising: (a) recombinant egg-white proteins consisting of a recombinant ovomucoid (rOVD) and a recombinant ovalbumin (rOVA); (b) one or more gelation/thickening agents; (c) a salt and/or another flavoring agent; (d) a lipid component; and (e) water; wherein a weight ratio of recombinant egg-white proteins to lipid component is greater than 1:1.

Embodiment 2: The composition of Embodiment 1, wherein a weight ratio of rOVD and rOVA is from about 1:50 to about 2:1.

Embodiment 3: The composition of Embodiment 1 or Embodiment 2, wherein the weight percent of protein to composition is greater than about 2% on a w/w basis.

Embodiment 4: The composition of any one of Embodiments 1 to 3, wherein the weight percent of protein to composition is less than about 15% on a w/w basis.

Embodiment 5: The composition of any one of Embodiments 1 to 4, wherein the composition lacks any animal-derived substances or any animal-derived components.

Embodiment 6: The composition of any one of Embodiments 1 to 5, wherein a weight ratio of rOVD and rOVA is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1.

Embodiment 7: The composition of any one of Embodiments 1 to 6 wherein the weight percent of rOVA to composition is from about 2% to about 10% on a w/w basis.

Embodiment 8: The composition of any one of Embodiments 1 to 7, wherein the rOVA has one or more N-linked glycosylation sites having mannose linked to an N-acetyl glucosamine, and wherein the N-linked glycosylation sites lack galactose.

Embodiment 9: The composition of any one of Embodiments 1 to 8, wherein the rOVA has at least 80%, 85%, 90%, 92%, 95%, 96%, 97%, 98%, 99% of 100% sequence identity to any one of SEQ ID NO: 45 to SEQ ID NO: 118.

Embodiment 10: The composition of any one of Embodiments 1 to 9, wherein the weight percent of rOVD to composition is from about 0.15% to about 4.5% on a w/w basis.

Embodiment 11: The composition of any one of Embodiments 1 to 10, wherein the rOVD comprises a glycosylation pattern that differs from the glycosylation pattern of a native chicken ovomucoid.

Embodiment 12: The composition of any one of Embodiments 1 to 11, wherein the rOVD comprises at least one glycosylated asparagine residue.

Embodiment 13: The composition of any one of Embodiments 1 to 12, wherein the rOVD is substantially devoid of N-linked mannosylation.

Embodiment 14: The composition of any one of Embodiments 1 to 13, wherein the rOVD has at least 80%, 85%, 90%, 92%, 95%, 96%, 97%, 98%, 99% of 100% sequence identity to any one of SEQ ID NO. 1 to SEQ ID NO: 44.

Embodiment 15: The composition of any one of Embodiments 1 to 14, wherein when the composition and a whole hen's egg are prepared as a scramble, the scrambled composition provides sensory attributes that are comparable to those of the scrambled whole hen's egg;
wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 16: The composition of any one of Embodiments 1 to 14, wherein when the composition and a composition comprising a protein component consisting of proteins obtained from a plant are prepared as a scramble, the scrambled composition provides better sensory attributes than those of a scrambled composition comprising a protein component consisting of proteins obtained from a plant;
wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 17: The composition of any one of Embodiments 1 to 16, wherein the amino acid profile of the recombinant egg-white proteins is closer to a whole hen's egg than the amino acid profile of a protein component consisting of proteins obtained from a plant.

Embodiment 18: The composition of any one of Embodiments 1 to 17, wherein the nutrition value provided by amino acids of the recombinant egg-white proteins is closer to a whole hen's egg than the nutrition value provided by amino acids of a protein component consisting of proteins obtained from a plant.

Embodiment 19: The composition of any one of Embodiments 1 to 18, wherein the recombinant egg-white protein comprises a fraction of cysteine, methionine, and/or lysine amino acids that is closer to the fraction in a whole hen's egg than the fraction in a protein component consisting of proteins obtained from a plant.

Embodiment 20: The composition of any one of Embodiments 1 to 19, wherein the recombinant egg-white protein comprises a larger fraction of cysteine, methionine, and/or lysine amino acids than the fraction in a composition comprising a protein component consisting of proteins obtained from a plant.

Embodiment 21: The composition of any one of Embodiments 1 to 20, wherein the recombinant egg-white protein comprises a fraction of cysteine and methionine amino acids closer to the fraction in a whole hen's egg than the fraction in a protein component consisting of proteins obtained from a plant.

Embodiment 22: The composition of any one of Embodiments 19 to 21, wherein the fraction of cysteine, methionine, and/or lysine amino acids in the recombinant egg-white proteins provides, in part, a flavor and/or smell that approximates the flavor and/or smell of a whole hen's egg.

Embodiment 23: The composition of any one of Embodiments 19 to 22, wherein the fraction of cysteine, methionine, and/or lysine amino acids in the recombinant egg-white proteins provides, in part, a flavor and/or smell that is superior to the flavor and/or smell of composition comprising a protein component consisting of proteins obtained from a plant.

Embodiment 24: The composition of any one of Embodiments 16 to 23, wherein the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

Embodiment 25: The composition of any one of Embodiments 16 to 24, wherein the proteins obtained from a plant comprise or consist of chickpea protein and mung bean protein or the proteins obtained from a plant comprise or consist of lupin bean protein and pea protein.

Embodiment 26: A powdered whole egg substitute composition comprising: (a) recombinant egg-white proteins consisting of a recombinant ovomucoid (rOVD) and a recombinant ovalbumin (rOVA); (b) one or more gelation/thickening agents; (c) a salt and/or another flavoring agent; and (d) a lipid component; wherein a weight ratio of recombinant egg-white proteins to lipid component is greater than 1:1.

Embodiment 27: The composition of Embodiment 26, wherein a weight ratio of rOVD and rOVA is from about 1:50 to about 2:1.

Embodiment 28: The composition of Embodiment 26 or Embodiment 27, wherein the weight percent of protein to composition is greater than about 10% on a w/w basis.

Embodiment 29: The composition of any one of Embodiments 26 to 28, wherein the weight percent of protein to composition is less than about 95% on a w/w basis.

Embodiment 30: The composition of any one of Embodiments 26 to 29, wherein the composition lacks any animal-derived substances or any animal-derived components.

Embodiment 31: The composition of any one of Embodiments 26 to 30, wherein a weight ratio of rOVD and rOVA is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1.

Embodiment 32: The composition of any one of Embodiments 26 to 31 wherein the weight percent of rOVA to composition is from about 9% to about 86% on a w/w basis.

Embodiment 33: The composition of any one of Embodiments 26 to 32, wherein the rOVA has one or more N-linked glycosylation sites having mannose linked to an N-acetyl glucosamine, and wherein the N-linked glycosylation sites lack galactose.

Embodiment 34: The composition of any one of Embodiments 26 to 33, wherein the rOVA has at least 80%, 85%, 90%, 92%, 95%, 96%, 97%, 98%, 99% of 100% sequence identity to any one of SEQ ID NO: 45 to SEQ ID NO: 118.

Embodiment 35: The composition of any one of Embodiments 26 to 34, wherein the weight percent of rOVD to composition is from about 0.6% to about 50% on a w/w basis.

Embodiment 36: The composition of any one of Embodiments 26 to 35, wherein the rOVD comprises a glycosylation pattern that differs from the glycosylation pattern of a native chicken ovomucoid.

Embodiment 37: The composition of any one of Embodiments 26 to 36, wherein the rOVD comprises at least one glycosylated asparagine residue.

Embodiment 38: The composition of any one of Embodiments 26 to 37, wherein the rOVD is substantially devoid of N-linked mannosylation.

Embodiment 39: The composition of any one of Embodiments 26 to 38, wherein the rOVD has at least 80%, 85%, 90%, 92%, 95%, 96%, 97%, 98%, 99% of 100% sequence identity to any one of SEQ ID NO. 1 to SEQ ID NO: 44.

Embodiment 40: The composition of any one of Embodiments 26 to 39, wherein when the composition is combined with a liquid to form a liquid whole egg substitute composition, and when the liquid whole egg substitute composition and a whole hen's egg are prepared as a scramble, the scrambled whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 41: The composition of any one of Embodiments 26 to 40, wherein when the composition is combined with a liquid to form a liquid whole egg substitute composition, and when the liquid whole egg substitute composition and a liquid composition comprising a protein component consisting of proteins obtained from a plant are prepared as a scramble, the scrambled whole egg substitute composition provides better sensory attributes than those of a scrambled composition comprising a protein component consisting of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 42: The composition of any one of Embodiments 26 to 41, wherein the amino acid profile of the recombinant egg-white proteins is closer to a whole hen's egg than the amino acid profile of a protein component consisting of proteins obtained from a plant.

Embodiment 43: The composition of any one of Embodiments 26 to 42, wherein the nutrition value provided by amino acids of the recombinant egg-white proteins is closer to a whole hen's egg than the nutrition value provided by amino acids of a protein component consisting of proteins obtained from a plant.

Embodiment 44: The composition of any one of Embodiments 26 to 43, wherein the recombinant egg-white protein comprises a fraction of cysteine, methionine, and/or lysine amino acids that is closer to the fraction in a whole hen's egg than the fraction in a protein component consisting of proteins obtained from a plant.

Embodiment 45: The composition of any one of Embodiments 26 to 44, wherein the recombinant egg-white protein comprises a larger fraction of cysteine, methionine, and/or lysine amino acids than the fraction in a composition comprising a protein component consisting of proteins obtained from a plant.

Embodiment 46: The composition of any one of Embodiments 26 to 45, wherein the recombinant egg-white protein comprises a fraction of cysteine and methionine amino acids closer to the fraction in a whole hen's egg than the fraction in a protein component consisting of proteins obtained from a plant.

Embodiment 47: The composition of any one of Embodiments 44 to 46, wherein the fraction of cysteine, methionine, and/or lysine amino acids in the recombinant egg-white proteins provides, in part, a flavor and/or smell that approximates the flavor and/or smell of a whole hen's egg.

Embodiment 48: The composition of any one of Embodiments 44 to 47, wherein the fraction of cysteine, methionine, and/or lysine amino acids in the recombinant egg-white proteins provides, in part, a flavor and/or smell that is superior to the flavor and/or smell of composition comprising a protein component consisting of proteins obtained from a plant.

Embodiment 49: The composition of any one of Embodiments 41 to 48, wherein the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

Embodiment 50: The composition of any one of Embodiments 41 to 49, wherein the proteins obtained from a plant comprise or consist of chickpea protein and mung bean protein or the proteins obtained from a plant comprise or consist of lupin bean protein and pea protein.

Embodiment 51: A liquid whole egg substitute composition comprising: (a) recombinant egg-white proteins comprising a recombinant ovomucoid (rOVD) and/or a recombinant ovalbumin (rOVA); (b) one or more gelation/thickening agents; (c) a salt and/or another flavoring agent; (d) a lipid component; and (e) water; wherein a weight ratio of recombinant egg-white proteins to lipid component is greater than 1:1.

Embodiment 52: The composition of Embodiment 51, wherein a weight ratio of rOVD and rOVA is from about 1:50 to about 2:1.

Embodiment 53: The composition of Embodiment 51 or Embodiment 52, wherein the weight percent of protein to composition is greater than about 2% on a w/w basis.

Embodiment 54: The composition of any one of Embodiments 51 to 53, wherein the weight percent of protein to composition is less than about 20% on a w/w basis.

Embodiment 55: The composition of any one of Embodiments 51 to 54, wherein the composition lacks any animal-derived substances or any animal-derived components.

Embodiment 56: The composition of any one of Embodiments 51 to 55, wherein a weight ratio of rOVD and rOVA is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1.

Embodiment 57: The composition of any one of Embodiments 51 to 56 wherein the weight percent of rOVA to composition is from about 2% to about 10% on a w/w basis.

Embodiment 58: The composition of any one of Embodiments 51 to 57, wherein the rOVA has one or more N-linked glycosylation sites having mannose linked to an N-acetyl glucosamine, and wherein the N-linked glycosylation sites lack galactose.

Embodiment 59: The composition of any one of Embodiments 51 to 58, wherein the rOVA has at least 80%, 85%, 90%, 92%, 95%, 96%, 97%, 98%, 99% of 100% sequence identity to any one of SEQ ID NO: 45 to SEQ ID NO: 118.

Embodiment 60: The composition of any one of Embodiments 51 to 59, wherein the weight percent of rOVD to composition is from about 0.15% to about 4.5% on a w/w basis.

Embodiment 61: The composition of any one of Embodiments 51 to 60, wherein the rOVD comprises a glycosylation pattern that differs from the glycosylation pattern of a native chicken ovomucoid.

Embodiment 62: The composition of any one of Embodiments 51 to 61, wherein the rOVD comprises at least one glycosylated asparagine residue.

Embodiment 63: The composition of any one of Embodiments 51 to 62, wherein the rOVD is substantially devoid of N-linked mannosylation.

Embodiment 64: The composition of any one of Embodiments 51 to 63, wherein the rOVD has at least 80%, 85%, 90%, 92%, 95%, 96%, 97%, 98%, 99% of 100% sequence identity to any one of SEQ ID NO. 1 to SEQ ID NO: 44.

Embodiment 65: The composition of any one of Embodiments 51 to 64, wherein when the composition and a whole hen's egg are prepared as a scramble, the scrambled composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 66: The composition of any one of Embodiments 51 to 64, wherein when the composition and a composition comprising a protein component consisting of proteins obtained from a plant are prepared as a scramble, the scrambled composition provides better sensory attributes than those of a scrambled composition comprising a protein component consisting of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 67: The composition of any one of Embodiments 51 to 66, wherein the amino acid profile of the recombinant egg-white proteins is closer to a whole hen's egg than the amino acid profile of a protein component consisting of proteins obtained from a plant.

Embodiment 68: The composition of any one of Embodiments 51 to 67, wherein the nutrition value provided by amino acids of the recombinant egg-white proteins is closer to a whole hen's egg than the nutrition value provided by amino acids of a protein component consisting of proteins obtained from a plant.

Embodiment 69: The composition of any one of Embodiments 51 to 68, wherein the recombinant egg-white protein comprises a fraction of cysteine, methionine, and/or lysine amino acids that is closer to the fraction in a whole hen's egg than the fraction in a protein component consisting of proteins obtained from a plant.

Embodiment 70: The composition of any one of Embodiments 51 to 69, wherein the recombinant egg-white protein comprises a larger fraction of cysteine, methionine, and/or lysine amino acids than the fraction in a composition comprising a protein component consisting of proteins obtained from a plant.

Embodiment 71: The composition of any one of Embodiments 69 to 70, wherein the fraction of cysteine, methionine, and/or lysine amino acids in the recombinant egg-white proteins provides, in part, a flavor and/or smell that approximates the flavor and/or smell of a whole hen's egg.

Embodiment 72: The composition of any one of Embodiments 69 to 71, wherein the fraction of cysteine, methionine, and/or lysine amino acids in the recombinant egg-white proteins provides, in part, a flavor and/or smell that is superior to the flavor and/or smell of composition comprising a protein component consisting of proteins obtained from a plant.

Embodiment 73: The composition of any one of Embodiments 51 to 72, wherein the composition further comprises one or more proteins obtained from a plant.

Embodiment 74: The composition of any one of Embodiments 66 to 73, wherein the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

Embodiment 75: The composition of any one of Embodiments 66 to 74, wherein the proteins obtained from a plant comprise or consist of chickpea protein and mung bean protein or the proteins obtained from a plant comprise or consist of lupin bean protein and pea protein.

Embodiment 76: The composition of any one of Embodiments 51 to 75, wherein the recombinant egg-white proteins further comprises recombinant lysozyme (rOVL).

Embodiment 77: The composition of Embodiment 76, wherein the weight percent of rOVL to composition is from about 0.1% to about 5% on a w/w basis.

Embodiment 78: The composition of Embodiment 76 or Embodiment 77, wherein the rOVL is a recombinant chicken egg white lysozyme (cOVL) or a recombinant goose lysozyme (gOVL).

Embodiment 79: The composition of any one of Embodiments 76 to 718, wherein the rOVL has at least 80%, 85%, 90%, 92%, 95%, 96%, 97%, 98%, 99% of 100% sequence identity to any one of SEQ ID NO: 119 to SEQ ID NO: 125.

Embodiment 80: A powdered whole egg substitute composition comprising: (a) recombinant egg-white proteins comprising a recombinant ovomucoid (rOVD) and/or a recombinant ovalbumin (rOVA); (b) one or more gelation/thickening agents; (c) a salt and/or another flavoring agent; and (d) a lipid component wherein a weight ratio of recombinant egg-white proteins to lipid component is greater than 1:1.

Embodiment 81: The composition of Embodiment 80, wherein a weight ratio of rOVD and rOVA is from about 1:50 to about 2:1.

Embodiment 82: The composition of Embodiment 80 or Embodiment 81, wherein the weight percent of protein to composition is greater than about 10% on a w/w basis.

Embodiment 83: The composition of any one of Embodiments 80 to 82, wherein the weight percent of protein to composition is less than about 95% on a w/w basis.

Embodiment 84: The composition of any one of Embodiments 80 to 83, wherein the composition lacks any animal-derived substances or any animal-derived components.

Embodiment 85: The composition of any one of Embodiments 80 to 84, wherein a weight ratio of rOVD and rOVA is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1.

Embodiment 86: The composition of any one of Embodiments 80 to 85 wherein the weight percent of rOVA to composition is from about 9% to about 86% on a w/w basis.

Embodiment 87: The composition of any one of Embodiments 80 to 86, wherein the rOVA has one or more N-linked glycosylation sites having mannose linked to an N-acetyl glucosamine, and wherein the N-linked glycosylation sites lack galactose.

Embodiment 88: The composition of any one of Embodiments 80 to 87, wherein the rOVA has at least 80%, 85%, 90%, 92%, 95%, 96%, 97%, 98%, 99% of 100% sequence identity to any one of SEQ ID NO: 45 to SEQ ID NO: 118.

Embodiment 89: The composition of any one of Embodiments 80 to 88, wherein the weight percent of rOVD to composition is from about 0.6% to about 50% on a w/w basis.

Embodiment 90: The composition of any one of Embodiments 80 to 89, wherein the rOVD comprises a glycosylation pattern that differs from the glycosylation pattern of a native chicken ovomucoid.

Embodiment 91: The composition of any one of Embodiments 80 to 90, wherein the rOVD comprises at least one glycosylated asparagine residue.

Embodiment 92: The composition of any one of Embodiments 80 to 91, wherein the rOVD is substantially devoid of N-linked mannosylation.

Embodiment 93: The composition of any one of Embodiments 80 to 92, wherein the rOVD has at least 80%, 85%, 90%, 92%, 95%, 96%, 97%, 98%, 99% of 100% sequence identity to any one of SEQ ID NO. 1 to SEQ ID NO: 44.

Embodiment 94: The composition of any one of Embodiments 80 to 93, wherein when the composition is combined with a liquid to form a liquid whole egg substitute composition, and when the liquid whole egg substitute composition and a whole hen's egg are prepared as a scramble, the scrambled whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 95: The composition of any one of Embodiments 80 to 94, wherein when the composition is combined with a liquid to form a liquid whole egg substitute composition, and when the liquid whole egg substitute composition and a liquid composition comprising a protein component consisting of proteins obtained from a plant are prepared as a scramble, the scrambled whole egg substitute composition provides better sensory attributes than those of a scrambled composition comprising a protein component consisting of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 96: The composition of any one of Embodiments 80 to 95, wherein the amino acid profile of the recombinant egg-white proteins is closer to a whole hen's egg than the amino acid profile of a protein component consisting of proteins obtained from a plant.

Embodiment 97: The composition of any one of Embodiments 80 to 96, wherein the nutrition value provided by amino acids of the recombinant egg-white proteins is closer to a whole hen's egg than the nutrition value provided by amino acids of a protein component consisting of proteins obtained from a plant.

Embodiment 98: The composition of any one of Embodiments 80 to 97, wherein the recombinant egg-white protein comprises a fraction of cysteine, methionine, and/or lysine amino acids that is closer to the fraction in a whole hen's egg than the fraction in a protein component consisting of proteins obtained from a plant.

Embodiment 99: The composition of any one of Embodiments 80 to 98, wherein the recombinant egg-white protein comprises a larger fraction of cysteine, methionine, and/or lysine amino acids than the fraction in a composition comprising a protein component consisting of proteins obtained from a plant.

Embodiment 100: The composition of any Embodiment 98 and Embodiment 99, wherein the fraction of cysteine, methionine, and/or lysine amino acids in the recombinant egg-white proteins provides, in part, a flavor and/or smell that approximates the flavor and/or smell of a whole hen's egg.

Embodiment 101: The composition of any one of Embodiments 98 to 100, wherein the fraction of cysteine, methionine, and/or lysine amino acids in the recombinant egg-white proteins provides, in part, a flavor and/or smell that is superior to the flavor and/or smell of composition comprising a protein component consisting of proteins obtained from a plant.

Embodiment 102: The composition of any one of Embodiments 80 to 101, wherein the composition further comprises one or more proteins obtained from a plant.

Embodiment 103: The composition of any one of Embodiments 95 to 102, wherein the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

Embodiment 104: The composition of any one of Embodiments 95 to 103, wherein the proteins obtained from a plant comprise or consist of chickpea protein and mung bean protein or the proteins obtained from a plant comprise or consist of lupin bean protein and pea protein.

Embodiment 105: The composition of any one of Embodiments 80 to 104, wherein the recombinant egg-white proteins further comprises recombinant lysozyme (rOVL).

Embodiment 106: The composition of Embodiment 105, wherein the weight percent of rOVL to composition is from about 0.1% to about 15% on a w/w or w/v basis.

Embodiment 107: The composition of Embodiment 105 or Embodiment 106, wherein the rOVL is a recombinant chicken egg white lysozyme (cOVL) or a recombinant goose lysozyme (gOVL).

Embodiment 108: The composition of any one of Embodiments 105 to 107, wherein the rOVL has at least 80%, 85%, 90%, 92%, 95%, 96%, 97%, 98%, 99% of 100% sequence identity to any one of SEQ ID NO: 119 to SEQ ID NO: 125.

Embodiment 109: The composition of any one of Embodiments 1 to 108, wherein the recombinant egg-white proteins are expressed in Pichia pastoris.

Embodiment 110: The composition of any one of Embodiments 1 to 109, wherein the one or more gelation agents comprises one or more polysaccharide-based hydrocolloids or protein-based hydrocolloids.

Embodiment 111: The composition of Embodiment 110, wherein the one or more polysaccharide or protein-based hydrocolloids comprises a beta-glucan, gellan gum (e.g., high acyl gellan gum and low acyl gellan gum), guar gum, locust bean gum, xanthan gum, carageenan (e.g., kappa carrageenan and iota carrageenan), alginate, sodium alginate, agar, gum arabic, lecithin, gelatin, pectin, Psyllium, corn starch, potato starch, rice starch, tapioca starch, modified starch, carboxy methylcellulose, methylcellulose, hydroxypropyl methylcullose, konjac gum, or transglutaminase.

Embodiment 112: The composition of Embodiment 110 or Embodiments 111, wherein the polysaccharide-based hydrocolloids comprises a beta-glucan.

Embodiment 113: The composition of any one of Embodiments 110 to 112, wherein the polysaccharide-based hydrocolloids comprises high acyl gellan gum or low acyl gellan gum.

Embodiment 114: The composition of any one of Embodiments 110 to 113, wherein the polysaccharide-based hydrocolloids comprises a beta-glucan and a gellan gum.

Embodiment 115: The composition of any one of Embodiments 110 to 114, wherein the weight percent of the one or more gelation agents to composition is from about 0.5% to about 5% on a w/w or w/v basis.

Embodiment 116: The composition of any one of Embodiments 1 to 115, wherein the salt comprises white salt, black salt, or Himalayan black salt (e.g., Rock salts (such as kala namak)) and/or comprises a Na+, Ca+2, K+, or Mg+2 cation, optionally, wherein the salt serves as a cross-linking agent.

Embodiment 117: The composition of Embodiment 115 or Embodiment 116, wherein the salt comprises Rock salts (such as kala namak).

Embodiment 118: The composition of any one of Embodiments 1 to 117, wherein the weight percent of the salt to composition is from about 0.1% to about 2% on a w/w or w/v basis.

Embodiment 119: The composition of any one of Embodiments 1 to 118, wherein the other flavoring agent comprises a natural or synthetic flavoring.

Embodiment 120: The composition of Embodiment 119, wherein the synthetic flavoring comprises synthetic egg yolk flavor.

Embodiment 121: The composition of any one of Embodiments 1 to 120, wherein the weight percent of the other flavoring agent to composition is from about 0.1% to about 5% on a w/w or w/v basis.

Embodiment 122: The composition of any one of Embodiments 1 to 121, wherein the lipid component comprises one or more saturated vegetable oils or unsaturated vegetable oils.

Embodiment 123: The composition of Embodiment 122, wherein the one or more saturated vegetable oils or unsaturated vegetable oils comprises coconut oil, palm oil, palm kernel oil, canola oil, soybean oil, corn oil, cottonseed oil, olive oil, flaxseed oil, sunflower oil, safflower oil, peanut oil, or avocado oil.

Embodiment 124: The composition of Embodiment 122 or Embodiment 123, wherein the one or more saturated vegetable oils or unsaturated vegetable oils are in their natural state or are chemically or enzymatically processed.

Embodiment 125: The composition of Embodiment 124, wherein the chemically or enzymatically processing produces an interesterified oil.

Embodiment 126: The composition of any one of Embodiments 122 to 125, wherein the saturated vegetable oils or unsaturated vegetable oil comprises one or more of coconut oil, palm oil, and palm kernel oil.

Embodiment 127: The composition of any one of Embodiments 122 to 126, wherein the saturated vegetable oils or unsaturated vegetable oil comprises two or more of coconut oil, palm oil, and palm kernel oil.

Embodiment 128: The composition of any one of Embodiments 122 to 127, wherein the saturated vegetable oils or unsaturated vegetable oil comprises each of coconut oil, palm oil, and palm kernel oil.

Embodiment 129: The composition of any one of Embodiments 1 to 128, wherein the weight percent of the lipid component to composition is from about 2% to about 15% on a w/w or w/v basis.

Embodiment 130: The composition of any one of Embodiments 1 to 129, wherein the composition further comprises one or more thickening agents.

Embodiment 131: The composition of Embodiment 130, wherein the one or more thickening agents comprises corn starch, potato starch, arrowroot starch, rice starch, tapioca starch, tapioca syrup, rice syrup, modified starch, carboxymethylcellulose, guar gum, locust bean gum, xanthan gum, carrageenan, gum Arabic, and Psyllium.

Embodiment 132: The composition of Embodiment 130 or 131, wherein the one or more thickening agents comprises one or more of tapioca syrup, Psyllium, and xanthan gum.

Embodiment 133: The composition of any one of Embodiments 130 to 132, wherein the one or more thickening agents comprises two or more of tapioca syrup, Psyllium, and xanthan gum.

Embodiment 134: The composition of any one of Embodiments 130 to 133, wherein the one or more thickening agents comprises each of tapioca syrup, Psyllium, and xanthan gum.

Embodiment 135: The composition of any one of Embodiments 130 to 134, wherein the weight percent of the one or more thickening agents to composition is from about 0.1% to about 30% on a w/w basis.

Embodiment 136: The composition of any one of Embodiments 1 to 135, wherein the composition further comprises one or more natural or synthetic coloring.

Embodiment 137: The composition of Embodiment 136, wherein the one or more natural or synthetic coloring is pineapple yellow.

Embodiment 138: The composition of Embodiment 136 or Embodiment 137, wherein the weight percent of the one or more natural or synthetic coloring to composition is from about 0.1% to about 2% on a w/w basis.

Embodiment 139: The composition of any one of Embodiments 1 to 138, wherein the composition further comprises one or more a natural emulsifiers or synthetic emulsifiers.

Embodiment 140: The composition of Embodiment 139, wherein the one or more a natural emulsifiers or synthetic emulsifiers comprises soy or sunflower lecithin, mono- and diglycerides, ethoxylated mono- and diglycerides, polyglycerol esters, sugar esters, polysorbate, and sorbitan.

Embodiment 141: The composition of Embodiment 139 or Embodiment 140, wherein the one or more a natural emulsifiers or synthetic emulsifiers comprises sunflower lecithin.

Embodiment 142: The composition of any one of Embodiments 139 to 141, wherein the weight percent of the one or more natural or synthetic coloring to composition is from about 0.1% to about 2% on a w/w basis.

Embodiment 143: The composition of any one of Embodiments 1 to 142, wherein the composition further comprises one or more dietary fiber-containing component comprises one or more of Psyllium husk fiber, Bamboo fiber, oat fiber, carrot fiber, flaxseed, chia seed, wheat fiber, pea fiber, potato fiber, apple fiber, citrus fiber, accacia fiber, and cellulose fiber.

Embodiment 144: The composition of Embodiment 143, wherein the dietary fiber-containing component is present in the substantially liquid mixture in a concentration from about 0.1% to about 10% on a weight per weight or weight per volume basis.

Embodiment 145: The composition of Embodiment 143 or Embodiment 144, wherein the dietary fiber-containing component comprises Psyllium husk fiber.

Embodiment 146: The composition of Embodiment 145, wherein the weight percent of the Psyllium husk fiber to composition is from about 0.1% to about 5% on a w/w or w/v basis.

Embodiment 147: The composition of Embodiment 146, wherein the weight percent of the Psyllium husk fiber to composition is about 0.7% on a w/w or w/v basis.

Embodiment 148: The composition of any one of Embodiments 1 to 147, wherein the composition further comprises a flour.

Embodiment 149: The composition of any one of Embodiments 1 to 148, wherein the composition further comprises a leavening agent.

Embodiment 150: The composition of Embodiment 149, wherein the leavening agent is baking powder, yeast or baking soda.

Embodiment 151: The composition of any one of Embodiments 1 to 150, wherein when the composition is a liquid, the composition further comprises a syrup component.

Embodiment 152: The composition of Embodiment 151, wherein the syrup component comprises honey, high fructose corn syrup, high maltose corn syrup, corn syrup (e.g., glucose-free corn syrup), simple syrup (e.g., comprising sucrose), sweet potato syrup, tapioca syrup, maple syrup, agave syrup, cane syrup, golden syrup, or brown rice syrup, or a combination thereof.

Embodiment 153: The composition of Embodiment 151 or Embodiment 152, wherein the weight percent of the syrup component to composition is from about 0.1% to about 5%, from about 0.3% to about 2%, or from about 0.5% to about 1.5% on a w/w or w/v basis.

Embodiment 154: The composition of any one of Embodiments 1 to 153, wherein when the composition is a liquid, the weight percent of the water to composition is from about 25% to about 90%, about 50% to about 85%, or from about 65% to about 80% on a w/w or w/v basis.

Embodiment 155: The composition of any one of Embodiments 1 to 154, wherein the composition has a shelf-life of greater than 3, 4, 5, 6, or 7 days at a refrigerated temperature of about 37° F.

Embodiment 156: A liquid whole egg substitute composition comprising: (a) recombinant egg-white proteins consisting of a recombinant ovomucoid (rOVD) and a recombinant ovalbumin (rOVA); (b) one or more gelation agents; (c) a salt and/or another flavoring agent; (d) a lipid component; (e) one or more thickening agents; (f) one or more natural or synthetic coloring; (g) one or more a natural emulsifiers or synthetic emulsifiers; and (h) water; wherein a weight ratio of recombinant egg-white proteins to lipid component is greater than 1:1.

Embodiment 157: The composition of Embodiment 156, wherein a weight ratio of rOVD and rOVA is from about 1:50 to about 2:1.

Embodiment 158: The composition of Embodiment 156 or Embodiment 157, wherein the weight percent of protein to composition is greater than about 2% on a w/w basis.

Embodiment 159: The composition of any one of Embodiments 156 to 158, wherein the weight percent of protein to composition is less than about 15% on a w/w basis.

Embodiment 160: The composition of any one of Embodiments 156 to 159, wherein the composition lacks any animal-derived substances or any animal-derived components.

Embodiment 161: The composition of any one of Embodiments 156 to 160, wherein a weight ratio of rOVD and rOVA is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1.

Embodiment 162: The composition of any one of Embodiments 156 to 161 wherein the weight percent of rOVA to composition is from about 2% to about 10% on a w/w basis.

Embodiment 163: The composition of any one of Embodiments 156 to 162, wherein the rOVA has one or more N-linked glycosylation sites having mannose linked to an N-acetyl glucosamine, and wherein the N-linked glycosylation sites lack galactose.

Embodiment 164: The composition of any one of Embodiments 156 to 163, wherein the rOVA has at least 80%, 85%, 90%, 92%, 95%, 96%, 97%, 98%, 99% of 100% sequence identity to any one of SEQ ID NO: 45 to SEQ ID NO: 118.

Embodiment 165: The composition of any one of Embodiments 156 to 164, wherein the weight percent of rOVD to composition is from about 0.15% to about 4.5% on a w/w basis.

Embodiment 166: The composition of any one of Embodiments 156 to 165, wherein the rOVD comprises a glycosylation pattern that differs from the glycosylation pattern of a native chicken ovomucoid.

Embodiment 167: The composition of any one of Embodiments 156 to 166, wherein the rOVD comprises at least one glycosylated asparagine residue.

Embodiment 168: The composition of any one of Embodiments 156 to 167, wherein the rOVD is substantially devoid of N-linked mannosylation.

Embodiment 169: The composition of any one of Embodiments 156 to 168, wherein the rOVD has at least 80%, 85%, 90%, 92%, 95%, 96%, 97%, 98%, 99% of 100% sequence identity to any one of SEQ ID NO. 1 to SEQ ID NO: 44.

Embodiment 170: The composition of any one of Embodiments 156 to 169, wherein when the composition and a whole hen's egg are prepared as a scramble, the scrambled composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 171: The composition of any one of Embodiments 156 to 170, wherein when the composition and a composition comprising a protein component consisting of proteins obtained from a plant are prepared as a scramble, the scrambled composition provides better sensory attributes than those of a scrambled composition comprising a protein component consisting of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 172: The composition of any one of Embodiments 156 to 171, wherein the amino acid profile of the recombinant egg-white proteins is closer to a whole hen's egg than the amino acid profile of a protein component consisting of proteins obtained from a plant.

Embodiment 173: The composition of any one of Embodiments 156 to 172, wherein the nutrition value provided by amino acids of the recombinant egg-white proteins is closer to a whole hen's egg than the nutrition value provided by amino acids of a protein component consisting of proteins obtained from a plant.

Embodiment 174: The composition of any one of Embodiments 156 to 173, wherein the recombinant egg-white protein comprises a fraction of cysteine, methionine, and/or lysine amino acids that is closer to the fraction in a whole hen's egg than the fraction in a protein component consisting of proteins obtained from a plant.

Embodiment 175: The composition of any one of Embodiments 156 to 174, wherein the recombinant egg-white protein comprises a larger fraction of cysteine, methionine, and/or lysine amino acids than the fraction in a composition comprising a protein component consisting of proteins obtained from a plant.

Embodiment 176: The composition of any one of Embodiment 174 or Embodiment 175, wherein the fraction of cysteine, methionine, and/or lysine amino acids in the recombinant egg-white proteins provides, in part, a flavor and/or smell that approximates the flavor and/or smell of a whole hen's egg.

Embodiment 177: The composition of any one of Embodiments 174 to 177, wherein the fraction of cysteine, methionine, and/or lysine amino acids in the recombinant egg-white proteins provides, in part, a flavor and/or smell that is superior to the flavor and/or smell of composition comprising a protein component consisting of proteins obtained from a plant.

Embodiment 178: The composition of any one of Embodiments 156 to 177, wherein the composition further comprises one or more proteins obtained from a plant.

Embodiment 179: The composition of any one of Embodiments 171 to 178, wherein the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

Embodiment 180: The composition of any one of Embodiments 171 to 179, wherein the proteins obtained from a plant comprise or consist of chickpea protein and mung bean protein or the proteins obtained from a plant comprise or consist of lupin bean protein and pea protein.

Embodiment 181: The composition of any one of Embodiments 156 to 180, wherein one or more gelation agents comprises a beta-glucan and/or a gellan gum.

Embodiment 182: The composition of any one of Embodiments 156 to 181, wherein the salt comprises Rock salts (such as kala namak).

Embodiment 183: The composition of any one of Embodiments 156 to 182, wherein the other flavoring agent comprises synthetic egg yolk flavor.

Embodiment 184: The composition of any one of Embodiments 156 to 183, wherein the lipid component comprises one or more, two more, or each of coconut oil, palm oil, and palm kernel oil.

Embodiment 185: The composition of any one of Embodiments 156 to 184, wherein the one or more thickening agents comprises one or more, two or more of, or each of tapioca syrup, Psyllium, and xanthan gum.

Embodiment 186: The composition of any one of Embodiments 156 to 185, wherein the one or more natural or synthetic coloring is pineapple yellow.

Embodiment 187: The composition of any one of Embodiments 156 to 186, wherein the one or more a natural emulsifiers or synthetic emulsifiers comprises sunflower lecithin.

Embodiment 188: A powdered whole egg substitute composition comprising: (a) recombinant egg-white proteins consisting of a recombinant ovomucoid (rOVD) and a recombinant ovalbumin (rOVA); (b) one or more gelation agents; (c) a salt and/or another flavoring agent; and (d) a lipid component; (e) one or more thickening agents; (f) one or more natural or synthetic coloring; and (g) one or more a natural emulsifiers or synthetic emulsifiers; wherein a weight ratio of recombinant egg-white proteins to lipid component is greater than 1:1.

Embodiment 189: The composition of Embodiment 188, wherein a weight ratio of rOVD and rOVA is from about 1:50 to about 2:1.

Embodiment 190: The composition of Embodiment 188 or Embodiment 189, wherein the weight percent of protein to composition is greater than about 10% on a w/w basis.

Embodiment 191: The composition of any one of Embodiments 188 to 190, wherein the weight percent of protein to composition is less than about 95% on a w/w basis.

Embodiment 192: The composition of any one of Embodiments 188 to 191, wherein the composition lacks any animal-derived substances or any animal-derived components.

Embodiment 193: The composition of any one of Embodiments 188 to 192, wherein a weight ratio of rOVD and rOVA is less than about 1:50, is less than about 1:40, is less than about 1:30, is less than about 1:20, is less than about 1:10, is less than about 1:5, is less than about 1:4, is less than about 1:3, is less than about 1:2, less than about 1:1, or is less than about 2:1.

Embodiment 194: The composition of any one of Embodiments 188 to 193 wherein the weight percent of rOVA to composition is from about 9% to about 86% on a w/w basis.

Embodiment 195: The composition of any one of Embodiments 188 to 194, wherein the rOVA has one or more N-linked glycosylation sites having mannose linked to an N-acetyl glucosamine, and wherein the N-linked glycosylation sites lack galactose.

Embodiment 196: The composition of any one of Embodiments 188 to 195, wherein the rOVA has at least 80%, 85%, 90%, 92%, 95%, 96%, 97%, 98%, 99% of 100% sequence identity to any one of SEQ ID NO: 45 to SEQ ID NO: 118.

Embodiment 197: The composition of any one of Embodiments 188 to 196, wherein the weight percent of rOVD to composition is from about 0.6% to about 50% on a w/w basis.

Embodiment 198: The composition of any one of Embodiments 188 to 197, wherein the rOVD comprises a glycosylation pattern that differs from the glycosylation pattern of a native chicken ovomucoid.

Embodiment 199: The composition of any one of Embodiments 188 to 198, wherein the rOVD comprises at least one glycosylated asparagine residue.

Embodiment 200: The composition of any one of Embodiments 188 to 199, wherein the rOVD is substantially devoid of N-linked mannosylation.

Embodiment 201: The composition of any one of Embodiments 188 to 200, wherein the rOVD has at least 80%, 85%, 90%, 92%, 95%, 96%, 97%, 98%, 99% of 100% sequence identity to any one of SEQ ID NO. 1 to SEQ ID NO: 44.

Embodiment 202: The composition of any one of Embodiments 188 to 201, wherein when the composition is combined with a liquid to form a liquid whole egg substitute composition, and when the liquid whole egg substitute composition and a whole hen's egg are prepared as a scramble, the scrambled whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 203: The composition of any one of Embodiments 188 to 202, wherein when the composition is combined with a liquid to form a liquid whole egg substitute composition, and when the liquid whole egg substitute composition and a liquid composition comprising a protein component consisting of proteins obtained from a plant are prepared as a scramble, the scrambled whole egg substitute composition provides better sensory attributes than those of a scrambled composition comprising a protein component consisting of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

Embodiment 204: The composition of any one of Embodiments 188 to 203, wherein the amino acid profile of the recombinant egg-white proteins is closer to a whole hen's egg than the amino acid profile of a protein component consisting of proteins obtained from a plant.

Embodiment 205: The composition of any one of Embodiments 188 to 204, wherein the nutrition value provided by amino acids of the recombinant egg-white proteins is closer to a whole hen's egg than the nutrition value provided by amino acids of a protein component consisting of proteins obtained from a plant.

Embodiment 206: The composition of any one of Embodiments 188 to 205, wherein the recombinant egg-white protein comprises a fraction of cysteine, methionine, and/or lysine amino acids that is closer to the fraction in a whole hen's egg than the fraction in a protein component consisting of proteins obtained from a plant.

Embodiment 207: The composition of any one of Embodiments 188 to 206, wherein the recombinant egg-white protein comprises a larger fraction of cysteine, methionine, and/or lysine amino acids than the fraction in a composition comprising a protein component consisting of proteins obtained from a plant.

Embodiment 208: The composition of any one of Embodiments 188 to 207, wherein the fraction of cysteine, methionine, and/or lysine amino acids in the recombinant egg-white proteins provides, in part, a flavor and/or smell that approximates the flavor and/or smell of a whole hen's egg.

Embodiment 209: The composition of any one of Embodiments 206 to 209, wherein the fraction of cysteine, methionine, and/or lysine amino acids in the recombinant egg-white proteins provides, in part, a flavor and/or smell that is superior to the flavor and/or smell of composition comprising a protein component consisting of proteins obtained from a plant.

Embodiment 210: The composition of any one of Embodiments 188 to 209, wherein the composition further comprises one or more proteins obtained from a plant.

Embodiment 211: The composition of any one of Embodiments 203 to 210, wherein the proteins obtained from a plant include at least one of chickpea protein, pumpkin protein, sunflower protein, mung bean protein, chia protein, sesame seed protein, flaxseed protein, tara protein, rice protein, fava bean protein mushroom protein, lupin bean protein, soy protein, and pea protein.

Embodiment 212: The composition of any one of Embodiments 203 to 211, wherein the proteins obtained from a plant comprise or consist of chickpea protein and mung bean protein or the proteins obtained from a plant comprise or consist of lupin bean protein and pea protein.

Embodiment 213: The composition of any one of Embodiments 188 to 212, wherein one or more gelation agents comprises a beta-glucan and/or a gellan gum.

Embodiment 214: The composition of any one of Embodiments 188 to 213, wherein the salt comprises Rock salts (such as kala namak).

Embodiment 215: The composition of any one of Embodiments 188 to 214, wherein the other flavoring agent comprises synthetic egg yolk flavor.

Embodiment 216: The composition of any one of Embodiments 188 to 215, wherein the lipid component comprises one or more, two more, or each of coconut oil, palm oil, and palm kernel oil.

Embodiment 217: The composition of any one of Embodiments 188 to 216, wherein the one or more thickening agents comprises one or more, two or more of, or each of tapioca syrup, Psyllium, and xanthan gum.

Embodiment 218: The composition of any one of Embodiments 188 to 217, wherein the one or more natural or synthetic coloring is pineapple yellow.

Embodiment 219: The composition of any one of Embodiments 188 to 218, wherein the one or more a natural emulsifiers or synthetic emulsifiers comprises sunflower lecithin.

Embodiment 220. Use of the composition of any preceding Embodiment as an ingredient in making an egg-less food product.

Embodiment 221: The use of Embodiment 220, wherein the egg-less food product is an egg-less vegan scramble.

Embodiment 222: The use of Embodiment 220, wherein the egg-less food product is a baked product selected from the group consisting of cake (e.g., pound cake, sponge cake, yellow cake, or angel food cake), cookie, bagel, biscuit, bread, muffin, crepe, cupcake, scone, pancake, macaron, macaroon, meringue, choux pastry, and soufflé; a batter; a beverage selected from the group consisting of smoothie, milkshake, “egg-nog”, and coffee beverage; a confectionary selected from a gummy, a taffy, a chocolate, or a nougat; a dessert product selected from the group consisting of a mousse, a cheesecake, a custard, a pudding, a popsicle, a frozen dessert, and an ice cream; a food emulsion; a meat analog food product selected from a burger, patty, sausage, hot dog, sliced deli meat, jerky, bacon, nugget, a ground meat-like composition, and a formed meat-like composition; a noodle; a pasta; a pet food; a sauce or dressing selected from the group consisting of salad dressing, mayonnaise, commercial mayonnaise substitutes, alfredo sauce, and hollandaise sauce; a snack food selected from a protein bar, a nutrition bar, or a granola bar; a yoghurt; an egg-wash; or egg-like dish selected from the group consisting of scramble, omelet, patty, soufflé, quiche, and frittata.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Expression Constructs, Transformation, Protein Purification and Processing

Two expression constructs were created for expression of OVD (SEQ ID NO: 1) in Pichia pastoris. The first construct included the Alcohol oxidase 1 (AOX1) promoter. An OVD coding sequenced was fused in-frame with the alpha mating factor signal sequence downstream of the promoter sequence. A transcriptional terminator from the AOX1 gene was placed downstream of the OVD sequence. The expression construct was placed into a Kpas-URA 3 vector.

A second expression construct was created containing the methanol-inducible DAS1 promoter (ATCC No. 28485) upstream of the alpha mating factor signal sequence fused in frame with a nucleic acid sequence encoding the same OVD protein sequence as in the first expression construct. A transcriptional terminator from the AOX1 gene was placed downstream of the OVD sequence.

In both expression constructs, the OVD sequence was that of chicken (Gallus gallus) having amino acid sequence of SEQ ID NO: 1.

Both expression constructs were transformed into Pichia pastoris. Successful integration of the two constructs were confirmed by genomic sequencing.

Fermentation: Recombinant OVD (rOVD) from each expression construct was produced in a bioreactor at ambient conditions. A seed train for the fermentation process began with the inoculation of shake flasks with liquid growth broth. The inoculated shake flasks were kept in a shaker after which the grown Pichia pastoris was transferred to a production scale reactor.

The culture was grown at 30° C., at a set pH and dissolved oxygen (DO). The culture was fed with a carbon source.

Secreted rOVD was purified by separating cells from the liquid growth broth, performing multiple filtration steps, performing chromatography using and drying the final protein product to produce pure rOVD powder.

### Example 2: Expression Construct, Transformation, Protein Purification and Processing

Three expression constructs were created for expression of a mature form of OVD (SEQ ID NO: 1) in Pichia pastoris. The first construct included the AOX1 promoter. An OVD coding sequenced was fused in-frame with the alpha mating factor signal sequence downstream of the promoter sequence (SEQ ID NO: 39). A transcriptional terminator from the AOX1 gene was placed downstream of the OVD sequence. The host cells had eleven copies of OVD, ten of which were in the hybrid promoter system, with five driven by a shortened pAOX1. The eleventh copy was driven by a full-sized pAOX1 promoter.

A second expression construct was created containing a nucleic acid encoding the P. pastoris transcription factor HAC1 under the control of a strong methanol-inducible promoter. A transcriptional terminator from the AOX1 gene was placed downstream of the HAC1 sequence.

A third expression construct was created encoding a fusion protein. The construct comprises a nucleic acid that encodes the first 48 residues of Pichia OCH1 protein fused to a catalytically active version of the Streptomyces coelicoflavus EndoH (SEQ ID NO.: 119) and under a strong methanol-inducible promoter, pPEX11. A transcriptional terminator from the AOX1 gene was placed downstream of the EndoH-OCH1 fusion protein sequence.

The P. pastoris strain was modified to remove cytoplasmic killer plasmids and then further modified to have a deletion in the AOX1 gene. This deletion generated a methanol-utilization slow (mutS) phenotype that reduces the strain's ability to consume methanol. This base strain was transformed with the three expression constructs.

Linear cassettes of methanol-inducible promoter: ScPrePro (Saccharomyces pre-pro sequence)::ovomucoid::AOX1term; linear cassettes of methanol-inducible promoter::HAC1::AOX1term; and a linear cassette of methanol-inducible promoter::EndoH-OCH1::AOX1term were introduced into the base P. pastoris strain using standard electroporation methods.

Fermentation: Recombinant OVD from each expression construct was produced in a bioreactor at ambient conditions. A seed train for the fermentation process began with the inoculation of shake flasks with liquid growth broth. The inoculated shake flasks were kept in a shaker after which the grown P. pastoris was transferred to a production-scale reactor.

The culture was grown at 30° C., at a set pH and dissolved oxygen (DO). The culture was fed with a carbon source.

To expand production, an rOVD P. pastoris seed strain is removed from cryo-storage and thawed to room temperature. Contents of the thawed seed vials are used to inoculate liquid seed culture media in baffled flasks which were grown at 30° C. in shaking incubators. These seed flasks are then transferred and grown in a series of larger and larger seed fermenters (number to vary depending on scale) containing a basal salt media, trace metals, and glucose. Temperature in the seed reactors are controlled at 30° C., pH at 5, and DO at 30%. pH is maintained by feeding ammonia hydroxide which also acts as a nitrogen source. Once sufficient cell mass is reached, the grown rOVD P. pastoris is inoculated in a production-scale reactor containing basal salt media, trace metals, and glucose. Like in the seed tanks, the culture is also controlled at 30° C., pH 5 and 30% DO throughout the process. pH is again maintained by feeding ammonia hydroxide. During the initial batch glucose phase, the culture is left to consume all glucose and subsequently-produced ethanol. Once the target cell density is achieved and glucose and ethanol concentrations are confirmed to be zero, the glucose fed-batch growth phase is initiated. In this phase, glucose is fed until the culture reaches a target cell density. Glucose is fed at a limiting rate to prevent ethanol from building up in the presence of non-zero glucose concentrations. In the final induction phase, the culture is co-fed glucose and methanol which induces it to produce rOVD. Glucose is fed at an amount to produce a desired growth rate, while methanol is fed to maintain the methanol concentration at 1% to ensure that expression is consistently induced. Regular samples are taken throughout the fermentation process for analyses of specific process parameters (e.g., cell density, glucose/methanol concentrations, product titer, and quality). After a designated amount of fermentation time, secreted rOVD is collected and transferred for downstream processing.

The rOVD products were purified by separating cells from the liquid growth broth, performing multiple filtration steps, performing chromatography, and/or drying the final protein product to produce pure rOVD powder.

Post-translation modification from the OCH1-EndoH fusion protein resulted in the removal of the alpha factor pre-pro sequence. N-terminal sequencing results showed imprecise cleavage of the N-terminal pro sequence by the Pichia host post-transcription machinery fusing an additional four amino acid residues (major) or 6 amino acid residues (minor) to the N-terminus of the produced rOVD (SEQ ID NO: 37) or (SEQ ID NO:38) in comparison to the amino acid sequence of mature OVD (SEQ ID NO:1).

The molecular weight of rOVD from Pichia was compared against native chicken ovomucoid (nOVD) using SDS-PAGE. The rOVD showed a difference in migration. To ascertain whether the difference in gel migration was due to differential post-translational glycosylation, deglycosylated native ovomucoid was treated with PNGase F, an enzyme that specifically deglycosylates proteins (BioLabs 2020), and compared to the rOVD sample. The deglycosylated native ovomucoid (nOVD+PNGaseF) displayed the same band patterns and molecular weight as three rOVD samples tested (FIG. 1C). The difference in glycosylation is attributed to the action of the OCH1-EndoH in the Pichia strain, such that rOVD has only the core N-acetylglucosamine unit attached to the Asn residue instead of the complex branched glycosylation (that includes mannose) of nOVD from chicken egg white (FIG. 1A and FIG. 1B).

Mass spectrometry analysis of rOVD expressed in Pichia without EndoH is shown to have eight different N-glycan structures (FIG. 1B). The structures include Man9 GlcNAc2, Man9 GlcNAc2 Hex, Man9 GlcNAc2Hex2, Man9 GlcNAc2Hex3, Man9 GlcNAc2Hex4, Man9 GlcNAc2 Hex5,v Man9 GlcNAc2Hex6, and Man9 GlcNAc2 Hex7. Table 2 below shows the percentage of N-linked glycans on the rOVD sample produced without endoglycosidase treatment.

TABLE 2
N-linked glycans from sample detected by MALDI TOF/TOF MS.

| Permethylated | Text description of | |
|---|---|---|
| mass (m/z)¹ | structures | Percentage |
| 2396.2 | Man₉ GlcNAc₂ | 5.6 |
| 2600.3 | Man₉ GlcNAc₂ Hex | 25.1 |
| 2804.4 | Man₉ GlcNAc₂ Hex₂ | 31.6 |
| 3008.5 | Man₉ GlcNAc₂ Hex₃ | 18.2 |
| 3212.6 | Man₉ GlcNAc₂ Hex₄ | 6.0 |
| 3416.7 | Man₉ GlcNAc₂ Hex₅ | 7.2 |
| 3620.8 | Man₉ GlcNAc₂ Hex₆ | 3.8 |
| 3824.9 | Man₉ GlcNAc₂ Hex₇ | 2.6 |

### Example 3: Comparison of Bovine Trypsin Inhibitory Activity

rOVD as produced in Example 2 was utilized in this Example. The trypsin inhibition activity was compared between native OVD (nOVD) and recombinant OVD (rOVD) in a standard assay (AACC #22-40.01) using bovine trypsin. A comparison of rOVD with nOVD is shown in Table 3. One trypsin unit is arbitrarily defined as an increase of 0.01 absorbance unit at 410 nm per 10 ml of reaction mixture under the conditions of the assay. Trypsin inhibitor activity is expressed in terms of trypsin inhibitor units (TIU). Three different batches of rOVD (samples 1-3) were compared to a native chicken ovomucoid.

TABLE 3
Comparison of trypsin inhibition activity

| Product | Trypsin inhibition activity |
|---|---|
| rOVD1 | 8190 TIU/g |
| rOVD2 | 8180 TIU/g |
| rOVD3 | 8649 TIU/g |
| Native chicken Ovomucoid | 13721 TIU/g |

### Example 4: Comparison of In Vitro Digestibility

The in vitro digestibility of rOVD samples was measured using the Protein Digestibility Assay procedure (Megazyme, Medallion Labs). A comparison of rOVD samples with nOVD is shown in Table 4. The data demonstrates equivalent in vitro digestibility between native ovomucoid and rOVD.

TABLE 4
Comparison in vitro digestibility

| Product | In-vitro digestibility |
|---|---|
| rOVD1 | 93% |
| rOVD2 | 93% |
| rOVD3 | 93% |
| Native chicken Ovomucoid | 92% |

### Example 5: Ovomucoid Specifications

Based upon the characterization of the produced rOVD compositions and the properties of native chicken ovomucoid, product specifications (Table 5) and quality control specifications (Table 6) were constructed for an rOVD of the present disclosure.

Protein percentages were measured using AOAC 2006. See, Protein (crude) in animal feed, combustion method, 990.03. In: Official methods of analysis of AOAC International. 18th ed. Gaithersburg: ASA-SSA Inc. and AOAC 2006. Proximate Analysis and Calculations Crude Protein Meat and Meat Products Including Pet Foods—item 80. In: Official methods of analysis Association of Analytical Communities, Gaithersburg, MD, 17th edition, Reference data: Method 992.15 (39.1.16); NFNAP; NITR; NT.

Moisture percentages were measured using Association of Official Analytical Chemists. 1995. In Official Methods of Analysis.

Carbohydrate percentages were measured using methods described in J AOAC Int. 2012 September-October; 95(5):1392-7.

Fat by acid hydrolysis were measured using AOAC International. 2012. Official Method Fat (crude) or ether extraction in pet food. Gravimetric method, 954.02. In: Official Methods of Analysis of AOAC International, 19th ed., AOAC International, Gaithersburg, MD, USA, 2012.

Standard plate count was measured using AOAC International. 2005. Aerobic plate count in foods, dry rehydratable film, method 990.12. AOAC International, 17th ed. Gaithersburg, MD. Yeast and mold counts were measured using AOAC Official Method 997.02. Yeast and Mold Counts in Foods Dry Rehydratable Film Method (Petrifilm™ Method) First Action 1997 Final Action 2000 Salmonella was measured using AOAC International. 2005. Salmonella in selected foods, BAX automated system, method 2003.09. In Official methods of analysis of AOAC International, 17th ed., AOAC International, Gaithersburg, MD. Total coliform was measured using AOAC International. 2005. E. coli count in foods, dry rehydratable film, method 991.14. In: Official methods of analysis of AOAC International, 17th ed. AOAC International, Gaithersburg, MD.

TABLE 5
Specification for Ovomucoid produced by P. pastoris DFB-003

| Physical properties | Specification | | |
|---|---|---|---|
| Source | Yeast fermentation-derived | | |
| Appearance | White to off-white amorphous powder | | |
| Solubility | Soluble in water | | |
| | Specification | | Method |
| Chemical Properties | | | |
| (in powder as is) | | | |
| Protein | >75% | | AOAC 990.03^{1a} |
| | | | AOAC 992.15^{1b} |
| Moisture | Maximum 10.0% | | AOAC 925.09² |
| Carbohydrate | Maximum 20% | | Calculated |
| Ash | Maximum 2.0% | | AOAC 942.05³ |
| Fat by Acid Hydrolysis | <0.1% | | AOAC 954.02⁴ |
| Hg | <1 | ppm | ICP-AES⁵ |
| Pb | <1 | ppm | ICP-AES⁵ |
| As | <1 | ppm | ICP-AES⁵ |
| Cd | <1 | ppm | ICP-AES⁵ |
| Microbial Properties | | | |
| (in powder as is) | | | |
| Standard Plate Count | <10000 | CFU/g | AOAC 990.12⁶ |
| Yeast & Mold | <100 | CFU/g | AOAC 997.02⁷ |
| Salmonella | Not Detected/25 g | | AOAC 2003.09⁸ |
| E. coli | Not Detected/25 g | | AOAC 991.14⁹ |
| Total coliform | ≤30 | CFU/g | AOAC 991.14⁹ |

TABLE 6
Quality control results for three lots of Ovomucoid
produced by P. pastoris DFB-003

| Analysis Parameter | Specification | | SOL19303 | SOL19317 | SOL19351 |
|---|---|---|---|---|---|
| Protein | >75% | | 75.31 | 75.06 | 79.94 |
| Protein (% dry weight powder) | >80% | | 82.2 | 82.5 | 87.8 |
| Moisture and Volatiles | <10% | | 8.4 | 9 | 9 |
| Carbohydrates, Calculated | <20% | | 15.53 | 15.28 | 11.06 |
| Ash | <2% | | 0.76 | 0.66 | <0.4 |
| Fat by Acid Hydrolysis | <0.1% | | <0.10 | <0.10 | <0.10 |
| Arsenic (As) | <1 | mg/kg | <0.010 | <0.010 | <0.010 |
| Mercury (Hg) | <1 | mg/kg | <0.010 | <0.010 | <0.010 |
| Lead (Pb) | <1 | mg/kg | 0.03 | 0.063 | 0.168 |
| Cadmium (Cd) | <1 | mg/kg | <0.010 | <0.010 | <0.010 |
| Aerobic Plate Count | <10000 | CFU/g | <10 | <10 | <10 |
| Molds | <100 | CFU/g | <10 | <10 | <10 |
| Yeast | <100 | CFU/g | <10 | <10 | <10 |

| | | | | |
|---|---|---|---|---|
| Salmonella | Not Detected/ | Not | Not | Not |
| | 25 g | Detected | Detected | Detected |
| Escherichia Coli | Not Detected/ | Not | Not | Not |
| | 25 g | Detected | Detected | Detected |
| Coliforms | less than or equal | <10 | <10 | <10 |
| | to 30 CFU/g | | | |
| Absence of source organism | Not detected */ | Not | Not | Not |
| from product | mg sample | detected | detected | detected |
| Absence of encoding DNA | Not detected **/ | Not | Not | Not |
| from product | mg sample | detected | detected | detected |

* Limit of detection for source organism = 11 CFU/mg sample
** Limit of detection for encoding DNA = 10 femtogram

### Example 6: Absence of Production Organism and DNA in rOVD Preparations

rOVD powder was plated on PGA plates and if samples yielded colonies, these were re-streaked and analyzed by PCR for the presence of the Pichia organism. This procedure was applied to three lots of rOVD powder produced from the recombinant strain. No manufacturing organism was detected in any of the lots (Table 6).

PCR analysis was used to confirm that no encoding pieces of recombinant DNA was present in the rOVD preparation using primers for the rOVD cassette. OVD plasmid DNA was used as a positive control, producing a 570 bp band corresponding the OVD PCR product. This band was absent in all three rOVD powder lots tested.

### Example 7: Fermentation and Purification of rOVD

An rOVD P. pastoris seed strain was removed from cryo-storage and thawed to room temperature. Contents of the thawed seed vials were used to inoculate liquid culture media in the primary fermenter and grown at process temperature until target cell density was reached. Then, the grown rOVD P. pastoris was transferred to a production-scale reactor. The culture was grown in the production bioreactor at target fermentation conditions and fed a series of substrates. The fermentation was analyzed for culture purity at multiple times during the process.

The recombinant OVD was purified by separating the cells from the liquid medium by centrifugation, followed by microfiltration. Fermentation broth was first brought to pH 3 and diluted with DI water. Cells were removed using bucket centrifugation. The collected supernatant was brought to pH 7 using sodium hydroxide and a 0.2 μm filtration was performed followed by diafiltration with five volumes of deionized water. The permeates of the 0.2 μm were adjusted to pH 5 and then concentrated via 5 kDa TFF membrane. The 5 kDa retentate was precipitated using 65% saturation ammonium sulfate. After salt addition, the pH was adjusted to pH 4-4.1 with phosphoric acid. The mixture was incubated with agitation at room temperature overnight. The next day, precipitates were spun down using bucket centrifugation. The rOVD precipitates were dissolved in DI water and pH adjusted to 5 using sodium hydroxide. The rOVD solution was then diafiltered and then the retentate was passed through 0.2 μm bottle filters.

A spray dryer was used to dehydrate the rOVD solution into rOVD powder.

### Example 8: Hydrogen Peroxide Treatment During rOVD Purification

Liquid rOVD was concentrated to 50-60 g/L using a 5 kDa TFF membrane. The rOVD solution was passed through a 0.2 μm filter to remove microbes. Hydrogen peroxide, an oxygen-generating agent, in an amount to equal 10% volume of the solution was slowly added to the rOVD solution while stirring. The mixture was incubated with agitation and monitored to ensure color change from a dark green-brown color before treatment to a pale-yellow color after treatment. After 1.5 hours, diafiltration was performed via 5 kDa TFF membrane with 5 volumes of DI water. The rOVD in the 5 kDa diafiltration retentate was precipitated using ammonium sulfate at 65% salt saturation at room temperature. After addition of salt, the pH was adjusted to pH4-4.1 with phosphoric acid. The mixture was incubated with agitation overnight to form precipitates. The next day, the precipitates were spun down using bucket centrifugation. The precipitates were removed, dissolved in deionized water and pH adjusted to 5 using sodium hydroxide. Five kDa TFF membranes were cleaned and diafiltration was performed using volumes of DI water until a retentate conductivity of less than 2.0 mS was achieved. The retentate was passed through 0.2 μm bottle filters. The filtered rOVD solution was then spray dried and stored.

### Example 9: Reprocessed rOVD Treated with Hydrogen Peroxide

OVD powder was dissolved in deionized water to 50-60 g/L and filtered through a hollow fiber 0.2 μm tangential flow filter, then through a 0.2 μm bottle filter. Hydrogen peroxide in an amount to provide a 10% solution was slowly stirred into the rOVD solution and incubated for thirty minutes. The treated solution was washed through a 5 kDa membrane using 5 volumes of DI water.

Ammonium sulfate was slowly added to the retentate solution and the pH changed to between 4 to 4.1 using phosphoric acid. After overnight incubation with medium agitation, the solution was centrifuged, and supernatants discarded. Precipitates were collected, dissolved in DI water, and brought to pH 5 using sodium hydroxide. The protein solution was desalted with a 5 kDa membrane and filtered through a 0.2 μm bottle filter. Then, the protein solution was spray dried to produce rOVD powder.

### Example 10: Preparation of Recombinant Ovalbumin

A Gallus gallus OVA coding sequence was fused in-frame with the alpha mating factor signal sequence downstream of the promoter sequence (SEQ ID NO:45). A promoter was placed upstream of the signal sequence OVA coding sequence and a transcriptional terminator was placed downstream of the OVA sequence. The expression construct was placed into a Kpas-URA 3 vector.

The expression constructs were transformed into Pichia pastoris. Successful integration was confirmed by genomic sequencing.

Fermentation: Recombinant OVA was produced in a bioreactor at ambient conditions. A seed train for the fermentation process begins with the inoculation of shake flasks with liquid growth broth using 2 ml cryovials of Pichia pastoris which are stored at −80° C. and thawed at room temperature prior to inoculation.

The inoculated shake flasks were kept in a shaker at 30° C. for 24 hours, after which the grown Pichia pastoris was transferred to a production scale reactor.

The culture was grown at 30° C., at a set pH and dissolved oxygen (DO). The culture was fed with a carbon source. At the end of the fermentation, the target OVA protein was harvested from the supernatant.

Cell debris was removed, protein was purified and lyophilized to a dry powder. The OVA produced was used in the examples described below.

### Example 11: Fermentation and Production of rOVA

Fermentation: Strains for fermenting recombinant OVA (rOVA) were each cultured in a bioreactor at ambient conditions. A seed train for the fermentation process began with the inoculation of shake flasks with liquid growth broth. The inoculated shake flasks were kept in a shaker after which the grown P. pastoris was transferred to a production-scale reactor.

To expand production, a seed vial of rOVA P. pastoris seed strain was removed from cryo-storage and thawed to room temperature. Contents of the thawed seed vials were used to inoculate liquid seed culture media in baffled flasks which were grown at 30° C. in shaking incubators. These seed flasks were then transferred and grown in a series of larger and larger seed fermenters (number to vary depending on scale) containing a basal salt media, trace metals, and glucose. Temperature in the seed reactors was controlled at 30° C., pH at 5, and dissolved oxygen (DO) at 30%. pH was maintained by feeding ammonia hydroxide, which also acted as a nitrogen source. Once sufficient cell mass was reached, the grown rOVA P. pastoris was inoculated into a production-scale reactor containing basal salt media, trace metals, and glucose.

Like in the seed tanks, the culture was also controlled at 30° C., pH5 and 30% DO throughout the process. pH was again maintained by feeding ammonia hydroxide. During the initial batch glucose phase, the culture was left to consume all glucose and subsequently-produced ethanol. Once the target cell density was achieved and glucose and ethanol concentrations were confirmed to be zero, the glucose fed-batch growth phase was initiated. In this phase, glucose was fed until the culture reached a target cell density. Glucose was fed at a limiting rate to prevent ethanol from building up in the presence of non-zero glucose concentrations. In the final induction phase, the culture was co-fed glucose and methanol which induced it to produce rOVA via the pAOX promoters. Glucose was fed at an amount to produce a desired growth rate, while methanol was fed to maintain the methanol concentration at 1% to ensure that expression was consistently induced. Regular samples were taken throughout the fermentation process for analyses of specific process parameters (e.g., cell density, glucose/methanol concentrations, product titer, and quality). After a designated amount of fermentation time, secreted rOVA was collected and transferred for downstream processing.

The fermentation broth containing the secreted rOVA was subjected to centrifugation at 12,000 rpm. The supernatant was clarified using microfiltration. To concentrate the protein and remove excess water, ultrafiltration at room temperature was used. An appropriately sized filter was used to retain the target rOVA while the compounds, salts, and water smaller than rOVA passed through the filter. To reduce the final salt content and conductivity in preparation for chromatography, the concentrated rOVA retentate was dialyzed at pH 3.5 until the final conductivity of the material was 1.7 mS/cm. The bulk of the purification was done using cation exchange chromatography at pH 3.5. Citrate buffer containing a high salt concentration of sodium chloride was used to elute the bound rOVA from the resin. To remove the excess salts, the eluant was finally dialyzed to make a final protein solution containing about 5-10% protein and 85-95% water. The final solution was sterilized by passing it through a 0.2 μm bioburden filter. The water was evaporated using a spray dryer/lyophilizer at appropriate temperatures to produce a final powder containing about 80% protein.

### Example 12: Preparation of Solubilized rOVA

In this example, hydrophobic recombinant chicken rOVA was solubilized and passed through a 0.2 μm filter.

Recombinant rOVA was purified through ion exchange chromatography at pH 3.5 and was found to be insoluble. Sodium hydroxide was added to the solution to change the pH to 12.5 and solubilize the rOVA. The rOVA solution at pH 12.5 was passed through a 0.2 μm filter. Following filtration, the pH was returned to 6.5 using hydrochloric acid and the rOVA was spray dried or lyophilized. This dried chicken rOVA was then used in the Examples below.

### Example 13: Glycosylation of Gallus gallus rOVA

In this example, Pichia-secreted rOVA was analyzed for glycosylation patterns.

Native ovalbumin (nOVA) has two potential N-linked glycosylation sites (FIG. 2A). A single site of glycosylation at Asn-292 is found in the egg white. MALDI-TOF analysis has shown that the typical glycans on native OVA are organized as (Man)5(GlcNAc)5(Gal)1 (FIG. 2A) (Harvey et al., 2000). Analysis of glycans on rOVA showed a typical glycosylation pattern shown in (FIG. 2B).

Pichia secreted chicken rOVA from the above Example was analyzed by gel electrophoresis migration and observed in three distinct forms (three white arrows pointing to rOVA in the “Input” lane below a) glycosylation-free, b) mono-glycosylated and c) di-glycosylated. Both the mono- and di-glycosylated glycosyl chains were cleaved from the mature rOVA protein using either of the endoglycanases EndoH or PNGaseF. Both the “denatured” or “native” deglycosylation protocols were used (as described in the NEB catalog). The green arrow indicates exogenous EndoH and the purple arrow indicates exogenous PNGaseF added to the in vitro reactions (FIG. 2C).

Pichia secreted chicken rOVA was subjected to standard analysis using Mass spectrometry. It was found to have five versions of N-linked Glycans (ManGlcNAc): high-mannose glycans of Man9 (˜40%), Man10 (˜47%) or Man11 (˜13%) type of N-glycan structures (FIG. 2D).

### Example 14: Comparison of Egg Protein Gelation Properties

In this example, recombinant chicken ovomucoid (rOVD) and recombinant chicken ovalbumin (rOVA) gels were made using the above-described examples. rOVA, rOVD, and combinations of rOVA and rOVD were compared to whole egg and fresh egg white in a gel egg system. Gel properties were measured for hardness, cohesiveness and chewiness. Table 7 shows the results of the experimentation.

Protein gels were prepared using simple protein and DI water matrix to better understand gelation properties of individual proteins and in combination.

The protein gels were prepared as follows: Heat the protein solutions to 85° C. for 30 min in a waterbath. Allow equilibration for 2 hours at room temperature. Measure the gel strength properties using Texture Profile Analysis (TPA) with a 36 mm cylindrical probe at a 50% compression target load and 5 g trigger load.

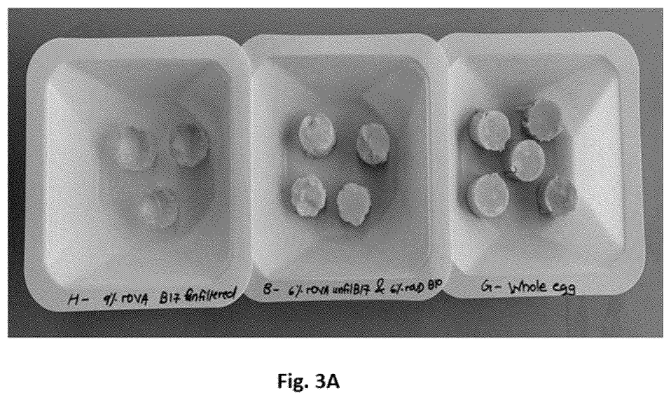

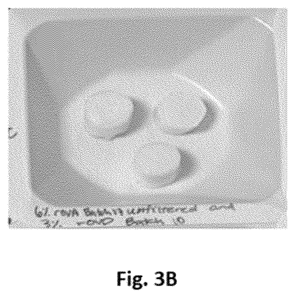

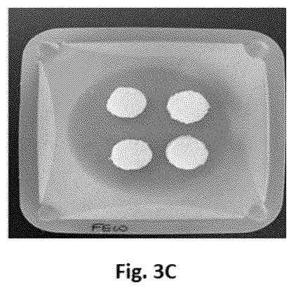

The photos in FIG. 3A shows gels formed from 9% rOVA, 6% rOVA and 6% rOVD, and whole egg, from left to right. The 9% rOVA, 6% rOVA and 6% rOVD gels are white in color, whereas the whole egg gel is yellow in color. FIG. 3B shows a gel formed from 6% rOVA and 3% rOVD, which is white in color. FIG. 3C shows a gel formed from fresh egg white, which is white in color.

TABLE 7
measured gel properties for different protein combinations

| | | | Chewiness |
|---|---|---|---|
| Protein Conc and Type | Hardness (g) | Cohesiveness | (mJ) |
| Fresh Egg White | 647 ± 5 abd | 0.68 ± 0.08 a | 21 ± 3 ab |
| Whole egg | 1202 ± 183 c | 0.63 ± 0.01 a | 36 ± 5 a |
| 9% rOVA | 282 ± 32 de | 0.2 ± 0.04 a | 2 ± 0.5 b |
| 12% rOVA | 647 ± 35 ad | 0.27 ± 0.02 a | 7 ± 0.5 bc |
| 6% rOVA + 3% rOVD | 148 ± 15 e | 0.32 ± 0.25 a | 2 ± 1 b |
| 6% rOVA + 6% rOVD | 845 ± 203 ac | 0.65 ± 0.14 a | 25 ± 11 ac |

| | |
|---|---|
| 6% rOVA | Viscous liquid; does not gel |
| 3% rOVD | Thin liquid; does not gel |
| 6% rOVD | Thin liquid; does not gel |
| 9% rOVD | Thin liquid; does not gel |
| 12% rOVD | Thin liquid; does not gel |

*Data that does not share the same letter within a given attribute is significantly different from each other (p < 0.05); based on HSD-Tukey analysis

In terms of gelling, an interesting result is that 6% rOVA protein solution does not gel, neither does 3% rOVD solution or 6% rOVD solution. However, combinations of these proteins, a. 6% rOVA and 3% rOVD; b. 6% rOVA and 6% rOVD gel well.

The hardness of 6% rOVA and 6% rOVD combination protein gel is similar to both fresh egg white and whole egg. 9% rOVA protein gel has similar hardness, cohesiveness and chewiness as compared to the protein combination of 6% rOVA and 3% rOVD.

In terms of hardness, cohesiveness and chewiness, 6% rOVA and 6% rOVD combination gel is similar to whole egg gel and 6% rOVA and 6% rOVD combination gel is similar to 12% rOVA gel, but whole egg gel is not similar to 12% rOVA in terms of hardness or chewiness.

12% rOVA is similar to fresh egg white in terms of hardness, and a combination of 6% rOVA and 6% rOVD is equivalent to fresh egg white in hardness, cohesiveness, and chewiness.

rOVA material gels are translucent, but combinations of rOVD and rOVA may allow the formation of a white opaque gel, such as that of the color of fresh egg white.

### Example 15: Comparison of Sensory Evaluation of Vegan Egg Scramble to Whole Egg Scramble

In this example, recombinant chicken ovomucoid (rOVD) and recombinant chicken ovalbumin (rOVA) were made using the above-described examples. Combinations of rOVA and rOVD were used to prepare a vegan egg scramble and was compared to fresh whole egg scramble as a control in a scramble preparation. The protein combinations used in the vegan scramble formulations was 6% rOVA and 3% rOVD and 6% rOVA and 6% rOVD.

Table 8 shows the ingredients used in the vegan scramble formulation. The dry ingredients were mixed together. The flavor was blended in oil separately and the blend was added to the dry mixture. Tapioca syrup with color and water were separately blended and then mixed with the flavor/oil/dry mixture. The above ingredients were then mixed carefully with a spatula until the powers were completely dissolved. Preheated griddle (Presto Liddle Griddle) was set to 225° F. and the mixture of all the ingredients was poured on to a pan, which was placed on the griddle. The mixture was then cooked for 3 minutes or until a cohesive cooked mass was formed.

5 panelists then compared the vegan egg scrambles and whole egg scramble in a sensory evaluation. A ‘difference from control’ test was used to differentiate the samples from the whole egg scramble in terms of overall quality, texture and flavor profile. Panelists were also asked to rate the samples in terms of likeability, along with the control. Table 9 shows the legend used to score and evaluate the samples. Table 10 shows the results of the sensory evaluation.

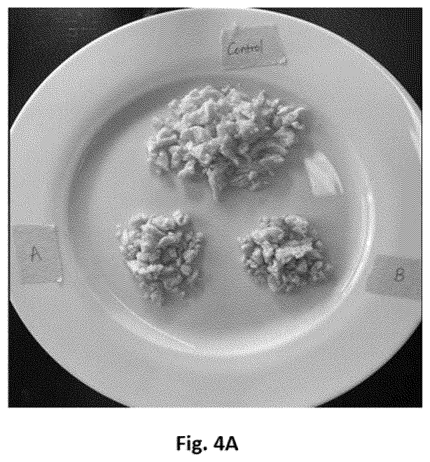

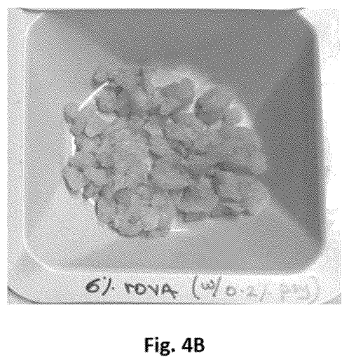

The photo in FIG. 4A shows the scramble compositions formed. The lower left composition (Sample A) was formed from 6% rOVA and 3% rOVD, the lower right composition (Sample B) is formed from 6% rOVA and 6% rOVD, and the upper composition was a control using fresh egg. Sample A and Sample B demonstrated a similar appearance in texture and color to that of the control. The photo in FIG. 4B shows the scramble composition of 6% rOVA, which formed a brittle gel with a less body, and a slightly different appearance than the control.

TABLE 8
Formulation used for vegan egg matrix

| | 6% rOVA and | 6% rOVA and |
|---|---|---|
| | 3% rOVD | 6% rOVD |
| Ingredients | Amount % | Amount % |
| rOVA | 7.00 | 7.00 |
| rOVD | 3.694581281 | 7.389162562 |
| Rock salts (such as kala namak) | 0.7 | 0.7 |
| Egg Flavor | 1 | 1 |
| High acyl Gellan gum | 0.51 | 0.51 |
| Coconut oil | 2.1 | 2.1 |
| Canola oil | 5 | 5 |
| Tapioca syrup | 0.3 | 0.3 |
| Psyllium | 0.2 | 0.2 |
| Pineapple yellow | 0.10 | 0.10 |
| DI | 79.39 | 75.70 |
| Total | 100.00 | 100.00 |

TABLE 9
Legend used to score and evaluate the samples:

| Score | Difference from Control | Score | Likeability test |
|---|---|---|---|
| 1 | no difference | 1 | dislike extremely |
| 2 | very slight difference | 2 | dislike very much |
| 3 | slight/moderate difference | 3 | dislike moderately |
| 4 | moderate difference | 4 | dislike slightly |
| 5 | moderate/large difference | 5 | neither like nor dislike |
| 6 | large difference | 6 | like slightly |
| 7 | very large difference | 7 | like moderately |
| | | 8 | like very much |
| | | 9 | like extremely |

TABLE 10
Results for sensory evaluation:

| | Difference from control | | | Likeability | | |
|---|---|---|---|---|---|---|
| Parameter/ | Overall | Flavor | Texture | | Control | |
| Sample | quality | quality | quality | Sample | (Whole egg) | Comments |
| 6% rOVA | 3 | 3 | 2 | 7 | 7 | Cohesive, |
| and 3% | Slight/ | Slight/ | Very slight | Like | Like | bouncy but |
| rOVD | moderate | moderate | difference | moderately | moderately | softer than |
| | difference | difference | | | | control. |
| | | | | | | Good color. |
| | | | | | | Not pasty |
| 6% rOVA | 4 | 4 | 3 | 6 | | Softer, |
| and 6% | Moderate | Moderate | Slight/ | Like | | grainy/curd |
| rOVD | difference | difference | moderate | slightly | | led texture, |
| | | | difference | | | pale color |

In terms of the difference from the control (whole egg scramble) the overall quality of the 6% rOVA and 3% rOVD was rated by the panelists as showing a slight/moderate difference as compared to the control, and the 6% rOVA and 6% rOVD was rated as showing a moderate difference as compared to the control.

In terms of the likeability of the vegan egg scrambles as compared to the control, the 6% rOVA and 3% rOVD egg scramble was rated by the panelists as having the same rating of likeability as the control.

### Example 16: Comparison of Sensory Evaluation of rOVD Vegan Scrambles and Other Plant-Based Vegan Scrambles

In this example, recombinant chicken ovomucoid (rOVD) were made using the above-described examples. rOVD was compared to other proteins, such as native OVD (nOVD), mungbean proteins, and chickpea protein alone as a control.

Ingredients in the formulations are listed in Table 11 and a list of the ingredients and their proportions used in the control and the other experimental samples with specific protein of interest are listed in Table 12. First, the dry ingredients (which includes the proteins of interest) were mixed together. The lecithin (used synonymously with “sunflower lecithin”) with the oil (used synonymously with “canola oil”) were blended together to make a blend. Then the blend was added to the dry mixture. Tapioca syrup was blended with water separately and then mixed with blend/mixture combination from the previous step. All the above ingredients were mixed with use of a stirrer for 20 seconds followed by a shear mixer for 1 minute. A preheated griddle (Presto Liddle Griddle) was set to 225° F. and the mixture of all the ingredients was poured on to a pan, which was placed on the griddle. The mixture was stirred while cooking and the mixture was cooked for 5-6 minutes or until a cohesive cooked mass was formed.

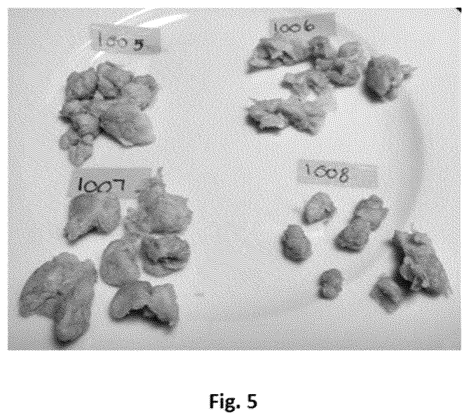

The photo in FIG. 5 shows multiple scramble compositions. Sample 1008 was a control scramble, which was formed from chickpea protein. Sample 1005 is a scramble formed using chickpea and mungbean. Sample 1006 shows a scramble formed using chickpea and nOVD. Sample 1007 shows a scramble formed from chickpea and rOVD.

TABLE 11
Ingredients used in vegan egg scramble preparations for their
evaluation against other vegan egg scramble preparations
Ingredients

```
Dry base ingredients: Chickpea protein, psyllium, calcium lactate,
baking powder, pretested agar, rock salts (such as kala namak)
Wet ingredients: Sunflower lecithin, canola oil, tapioca syrup, water
Proteins of interest to be tested:
    nOVD - 85% Protein content
    rOVD - 98% Protein Content
    Mung bean protein - 80% Protein content
```

TABLE 12
List of Ingredients and their proportions used in control formulation
and other experimental samples with specific protein of interest

| | Control | Chickpea- | Chickpea- | Chickpea- |
|---|---|---|---|---|
| | (Chickpea) | Mungbean | nOVD | rOVD |
| Ingredient | % | % | % | % |
| Lecithin | 0.2 | 0.2 | 0.2 | 0.2 |
| Canola oil | 5 | 5 | 5 | 5 |
| Chickpea protein | 20 | 10 | 10 | 10 |
| Test Protein | | 7.5 | 7.06 | 6.15 |
| Psyllium | 0.68 | 0.68 | 0.68 | 0.68 |
| Calcium lactate | 0.2 | 0.2 | 0.2 | 0.2 |
| baking powder | 0.75 | 0.75 | 0.75 | 0.75 |
| Agar | 0.25 | 0.25 | 0.25 | 0.25 |
| Rock salts (such | 0.75 | 0.75 | 0.75 | 0.75 |
| as kala namak) | | | | |
| Tapioca syrup | 1 | 1 | 1 | 1 |
| Water | 71.17 | 73.67 | 74.11 | 75.02 |
| Total | 100 | 100 | 100 | 100 |

Four in-house trained panelists participated in the sensory test quality descriptors, which included appearance, smell, taste/flavor, texture and overall liking in a nine-point scale from 1: Dislike extremely, 2: Dislike very much, 3: Dislike moderately, 4: Dislike slightly, 5: Neither like nor dislike, 6: Like slightly, 7: Like moderately, 8: Like very much, and 9: Like extremely. The results are shown in Table 13.

OVD fortified samples (both nOVD and rOVD) scored higher for texture/mouthfeel likeability, however, due to the large standard deviations, these differences were insignificant statistically. No statistically significant difference was observed by the panelists between all the samples for appearance, smell, taste, texture and overall liking.

TABLE 13
Likeability test results for vegan egg scramble based on a 9-point hedonic scale

| | Control | Chickpea/ | Chickpea/ | Chickpea/ | |
|---|---|---|---|---|---|
| | (Chickpea) | Mungbean | nOVD | rOVD | p Value |
| Appearance | *5.5 ± 1.29 a | 4.75 ± 1.50 a | 6 ± 1.63 a | 4.75 ± 0.96 a | Not |
| Likeability | | | | | significant |
| Smell | 5 ± 0.82 a | 4.25 ± 1.26 a | 5 ± 1.15 a | 4.25 ± 1.89 a | Not |
| Likeability | | | | | significant |
| Taste/Flavor | 5 ± 2.16 a | 4 ± 1.63 a | 4 ± 2.94 a | 3.5 ± 1.91 a | Not |
| Likeability | | | | | significant |
| Texture/Mouthfeel | 2.75 ± 1.26 a | 2.75 ± 0.50 a | 4 ± 2.45 a | 4 ± 2.16 a | Not |
| Likeability | | | | | significant |
| Overall | 4.25 ± 1.71 a | 3.75 ± 1.71 a | 4 ± 2.45 a | 4 ± 1.41 a | Not |
| Likeability | | | | | significant |

*Data that does not share the same letter for a specific attribute, is significantly different from each other (p < 0.05) based on Tukey-HSD test

The four panelists measured the appropriateness of the level of a specific attribute of the sample using a JAR (Just-About-Right) test. The four sample characteristics measured were color, saltiness, chewiness, and cohesiveness.

Table 14 demonstrates the results of the JAR scale for color attributes of the vegan egg scrambles. Chickpea-mungbean samples scored lower indicating a ‘not enough yellow’ color for the sample. Control, and OVD samples, had a JAR score. However, these differences between the samples were not statistically significant.

TABLE 14
JAR scale for color attribute of vegan egg scramble

| | Control | Chickpea/ | Chickpea/ | Chickpea/ |
|---|---|---|---|---|
| | (Chickpea) | Mungbean | nOVD | rOVD |
| *Too light | | 25% | | |
| Not yellow enough | 25% | 50% | 25% | 50% |
| JAR | 50% | 25% | 50% | 25% |
| Slightly more yellow | | | | |
| Too dark yellow | 25% | | 25% | 25% |
| Avg ± Std dev | **3.25 ± 1.26 a | 2 ± 0.82 a | 3.25 ± 1.26 a | 3 ± 1.41 a |

*JAR scale: 1: Too light, 2: Not yellow enough, 3: Just- About- Right, 4: Slightly more yellow, 5: Too dark yellow
**Data that does not share the same letter, is significantly different from each other (p < 0.05) based on Tukey-HSD test

Table 15 demonstrates the results of the JAR scale for saltiness of the vegan egg scramble. Panelists identified both the OVD samples (nOVD and rOVD) as saltier, and panelists commented that rOVD samples were yeasty savory while having higher perceived saltiness. Significant differences were observed in salt levels between chickpea-mungbean sample and rOVD sample, wherein the rOVD sample was higher in saltiness. However, no statistically significant differences were observed in control, chickpea-mungbean and nOVD samples.

TABLE 15
JAR scale for saltiness attribute of vegan egg scramble

| | Control | Chickpea/ | Chickpea/ | Chickpea/ |
|---|---|---|---|---|
| | (Chickpea) | Mungbean | nOVD | rOVD |
| *Too bland | | 25% | | |
| Not salty enough | 25% | | | |
| JAR | 75% | 75% | 25% | 25% |
| Slightly more salty | | | 75% | 25% |
| Too salty | | | | 50% |
| Avg ± Std dev | **2.75 ± 0.50 a | 2.50 ± 1 ab | 3.75 ± 0.5 abc | 4.25 ± 0.96 ac |

*JAR scale: 1: Too bland, 2: Not salty enough, 3: Just- About- Right, 4: Slightly more salty, 5: Too salty
**Data that does not share the same letter, is significantly different from each other (p < 0.05) based on Tukey-HSD test.

Table 16 demonstrates the results of the JAR scale for chewiness of the vegan egg scramble. panelists The panelists identified the control sample, chickpea mungbean sample and nOVD sample as soft, which is not a positive attribute. However, addition of rOVD to the matrix helped improve the chewiness profile and the panelists significantly scored it higher, indicating a JAR chewiness. Significant differences were observed between the inclusion of nOVD and rOVD, wherein rOVD performed better.

TABLE 16
JAR scale for chewiness attribute of vegan egg scramble

| | Control | Chickpea/ | Chickpea/ | Chickpea/ |
|---|---|---|---|---|
| | (Chickpea) | Mungbean | nOVD | rOVD |
| *Too soft | 100% | 100% | 50% | 25% |
| Not chewy enough | | | 50% | 50% |
| JAR | | | | |
| Slightly more chewy | | | | |
| Too chewy | | | | 25% |
| Avg ± Std dev | **1 ± 0.0 a | 1 ± 0.0 a | 1.15 ± 0.58 a | 2.75 ± 1.73 b |

*JAR scale: 1: Too soft, 2: Not chewy enough, 3: Just- About- Right, 4: Slightly more chewy, 5: Too chewy
**Data that does not share the same letter, is significantly different from each other (p < 0.05) based on Tukey-HSD test

Table 17 demonstrates the results of the JAR scale for cohesiveness. No significant differences were observed between all the samples for cohesiveness. Addition of OVD protein (both nOVD and rOVD) did not affect the cohesiveness observed in control samples.

TABLE 17
JAR scale for cohesiveness attribute of vegan egg scramble

| | Control | Chickpea/ | Chickpea/ | Chickpea/ |
|---|---|---|---|---|
| | (Chickpea) | Mungbean | nOVD | rOVD |
| *Too crumbly | | | | |
| Not cohesive enough | | | | |
| JAR | | | | |
| Slightly more cohesive | 25% | 25% | 75% | 50% |
| Too cohesive | 75% | 75% | 25% | 50% |
| Avg ± Std dev | **4.75 ± 0.50 a | 4.75 ± 0.50 a | 4.25 ± 0.5 a | 4.5 ± 0.58 a |

*JAR scale: 1: Too crumbly, 2: Not cohesive enough, 3: Just- About- Right, 4: Slightly more cohesive, 5: Too cohesive
**Data that does not share the same letter, is significantly different from each other (p < 0.05) based on Tukey-HSD test

Table 18 demonstrates the results of asking the panelists to measure intensities of bean like taste, chemical taste, chemical note and aftertaste attributes on a 5-point scale for each sample. The panelists identified very mild aftertaste in all samples. However, no significant differences were observed after addition of OVD protein. OVD fortified samples were also scored lower on bean like taste indicating a milder bean taste as compared to control, however, the differences were not statistically significant. All panelists observed that the samples were similar for mild chemical notes.

TABLE 18
Intensity test results for bean like taste, chemical note
and aftertaste intensity of vegan egg scramble samples

| | Control | Chickpea/ | Chickpea/ | Chickpea/ | |
|---|---|---|---|---|---|
| | (Chickpea) | Mungbean | nOVD | rOVD | p Value |
| Bean like | *2.25 ± 1.26 a | 1.75 ± 0.96 a | 3 ± 0.82 a | 2.75 ± 0.5 a | Not |
| Taste | Moderate beany | Moderate | Mild beany | Mild beany | significant |
| Intensity | note | beany note | note | note | |
| Chemical | 4.75 ± 0.5 a | 4.5 ± 0.58 a | 3.5 ± 1.29 a | 2.75 ± 1.26 a | Not |
| Note intensity | No chemical | very mild- no | mild- very mild | mild chemical | significant |
| | note | chemical note | chemical note | note | |
| Aftertaste | 3.25 ± 0.5 a | 3.25 ± 0.50 | 3.25 ± 1.50 a | 3.5 ± 1.29 a | Not |
| Intensity | very mild | very mild | very mild | very mild- | significant |
| | aftertaste | aftertaste | aftertaste | moderate aftertaste | |

*Data that does not share the same letter, is significantly different from each other (p < 0.05) based on Tukey-HSD test

Table 19 shows comments from the panelists that were made during their evaluation of the samples.

TABLE 19
Comments from the panelists

| Control | Chickpea/ | Chickpea/ | Chickpea/ |
|---|---|---|---|
| (Chickpea) | Mungbean | nOVD | rOVD |
| less flavor, | flavor is okay, | flavors are | little to no |
| needs little | sample is very | about right | smell, salty, not |
| more salt, | soft and mushy | chemical taste | mushy |
| sample very | beany chemical | texture close to | chemical taste |
| soft and very | taste | egg, but | and salty |
| mushy in | more beany and | slightly beany | gooey, salty, |
| mouth | mushy | and sour | beany |
| smells and taste | beany, | sticky but | chewier than |
| beany, mushy | cardboard | smooth texture, | nOVD sample, |
| beany taste. | aftertaste, | acidic/mustard | glossy |
| soft/mushy | mushy, sticky | like aftertaste | appearance, |
| cardboard | on teeth, had a | | acidic aftertaste |
| aftertaste, | powdery grainy | | |
| mushy | texture | | |

### Example 17: Illustrative Vegan Scramble

In this example, a vegan scramble was produced.

The vegan scramble was produced by performing the following steps: 1) Mix all the dry ingredients together (WIP 1); 2) Mix all the oil mix ingredients (WIP 2) separately; 3) Mix all the wet ingredients together (WIP 3) separately; 4) Mix in WIP 2 with WIP 1 with gentle mixing; 5) Stir in the WIP 3 with the WIP 1 & 2 combination; 6) Homogenize the mix well for upto 2 min; and 7) Heat process the sample to avoid any microbiological concerns (suggested temperature: 60 C for 3 min).

WIP 1, WIP 2, and WIP 3 were included in the amounts/proportions described in Table 20, below:

TABLE 20

| WIP No. # | WIP Description | Amount | % |
|---|---|---|---|
| WIP 1 | Dry Ingredients | 19.27 | 19.27 |
| WIP 2 | Oil mix | 5.2 | 5.2 |
| WIP 3 | Wet Ingredients | 75.53 | 75.53 |
| Total | | 100 | 100 |

WIP 1 included the dry ingredients in the amounts/proportions described in Table 21, below:

TABLE 21

| Ingredients | Amount (g) | % |
|---|---|---|
| Chickpea protein | 16.67 | 86.50752 |
| Psyllium | 0.68 | 3.528801 |
| Calcium lactate | 0.2 | 1.037883 |
| Baking powder | 0.75 | 3.89206 |
| Agar | 0.25 | 1.297353 |
| Flavor | 0.28 | 1.453036 |
| Rock salts (such as kala | 0.44 | 2.283342 |
| namak) | | |
| Total | 19.27 | |

WIP 2 included the oil mix in the amounts/proportions described in Table 22, below:

TABLE 22

| Ingredients | Amount (g) | % |
|---|---|---|
| Lecithin | 0.2 | 3.846154 |
| Canola oil | 5 | 96.15385 |
| Total | 5.2 | 100 |

WIP 3 included the wet ingredients in the amounts/proportions described in Table 23, below:

TABLE 23

| Ingredients | Amount (g) | % |
|---|---|---|
| Tapioca syrup | 1 | 1.323977 |
| Egg flavor | 3.3 | 4.369125 |
| Preservative | 0.55 | 0.728187 |
| Water | 70.68 | 93.57871 |
| Total | 75.53 | 100 |

The protein content of the vegan scramble described in this example was about 10 grams and approximately 5 grams of protein per 50 grams of vegan scramble.

### Example 18: Illustrative Vegan Scramble

In this example, a variety of vegan scrambles were produced and tested. Raters were given a series of different samples and then ask a set of structured questions about the sensory experiences of those samples. Several samples with different concentrations of each component were tested and exemplary samples are discussed below and in Table 24. The mixing instructions and ingredients used are as follows:

For R1-R6 the ingredients are provided in Table 24:
1. Mix together rOVD and rOVA with rock salt (Rock salts (such as kala namak)).
2. Blend water, tapioca Syrup, and yellow color and mix with Step 1 with spatula and stir bar.
3. Hydrate for at least 30 minutes.
4. Blend gums and mix with step 3.
5. Add flavor to Step 4.
6. Blend oil and sunflower lecithin together to make homogenous sample and mix with Step 5.
7. Before cooking, please make sure the scramble liquid mixture is homogeneous. Mix if needed. Cooking was performed at 350 F for scrambles comprising recombinant proteins and at 250 F for all others.

Experimenters added pepper roughly 0.5% black pepper to the seasoned example.

### Cooking Instructions:

For all samples, the experimenter uses the following procedure:
1. Use the Presto Liddle Griddle to cook the sample.
2. Set the griddle at target temperature and pour the mixture on to the pan.
3. Let the mix cook for 1.5 to 2 m. till a cohesive cooked mass is formed.
4. Samples were provided to 5-6 tasters (identity of the samples were not disclosed to the testers) and tasters were asked to review samples and rate them.

TABLE 24
Ingredients and concentrations tested (in %)

| ID | R1 | R2 | R3 | R4 | R5 | R6 |
|---|---|---|---|---|---|---|
| rOVA | 2.3 | 6.4 | 4.5 | 3.9 | 3.8 | 2.6 |
| rOVD | 3.1 | 4.2 | 1.4 | 1.1 | 1.8 | 5.8 |
| Rock salts (such | 0.2 | 1.1 | 0.7 | 1.2 | 0.3 | 0.4 |
| as kala namak) | | | | | | |
| Egg Yolk Flavor | 1.2 | 1.2 | 0.7 | 1 | 0.9 | 1.6 |
| (GS) (OS) | | | | | | |
| Coconut oil | 0 | 0 | 0 | 1.4 | 0.9 | 4.4 |
| Canola oil | 5.8 | 4.3 | 2.2 | 1.5 | 0.1 | 0 |
| Palm Oil | 0 | 0 | 1.7 | 0 | 1.4 | 0.9 |
| High acyl Gellan | 0.9 | 0.4 | 0.6 | 0.4 | 0.4 | 0.4 |
| gum | | | | | | |
| Beta-glucan | 0.2 | 0.6 | 1.7 | 0.4 | 0.6 | 1.5 |
| Tapioca syrup | 0.8 | 0.4 | 0.5 | 0.1 | 0.5 | 0.6 |
| DE27 | | | | | | |
| Psyllium | 0.6 | 0.8 | 0 | 0.9 | 0.5 | 0.1 |
| Pineapple yellow | 0.5 | 0 | 0.3 | 0.9 | 0.4 | 0.3 |
| Sunflower Lecithin | 0 | 0 | 0.7 | 0 | 1.3 | 1.1 |
| DI | 84.4 | 80.6 | 85 | 87.2 | 87.1 | 80.3 |

Tasters were asked to rate the samples R1-R6 for likeability and similarity to eggs. Per likeability the question asked was “Do you like sample X” and the ratings were based on the scale of Table 25:

TABLE 25
Likeability scale

| Numeric Score | Shown to Panelist |
|---|---|
| 0 | Dislike extremely |
| 1 | Dislike very much |
| 2 | Dislike moderately |
| 3 | Dislike slightly |
| 4 | Neither like nor dislike |
| 5 | Like slightly |
| 6 | Like moderately |
| 7 | Like very much |
| 8 | Like extremely |

Per similarity to eggs, the question asked was: “How different is sample X to the control?” Table 26 describes the scale:

TABLE 26
Similarity scale

| Numeric Score | Shown to Panelist |
|---|---|
| 6 | Not |
| 5 | Very slightly |
| 4 | Slight moderately |
| 3 | Moderately |
| 2 | Moderately largely |
| 1 | Largely |
| 0 | Very largely |

Results from tasters were as provided in Table 27 below:

TABLE 27
Tasting results

| ID | Likeability | Similarity |
|---|---|---|
| R1 | Dislike extremely | Very largely different |
| R2 | Like slightly | Moderately different |
| R3 | Like very much | Very slightly different |
| R4 | Like slightly | Slight moderately different |
| R5 | Like moderately | Moderately largely different |
| R6 | Like slightly | Moderately largely different |

### Example 19: Comparative Vegan Egg Scrambles

In this example, a variety of vegan scrambles were produced and tested. Ten to twelve raters were given a series of different samples and then asked a set of structured questions about the sensory experiences of those samples. This example considers “seasoned” and “unseasoned” scrambles separately. In both cases, the experimenter provided the hen's egg control first and identified it as “control” to the participant.

Two commercially-available, off-the-shelf products were used as comparative products. Those samples may already have contained unknown proprietary seasoning or common seasoning present. Therefore, for this first set of experiments, the experimenter added salt and pepper to simulate as if a consumer would be eating these scrambles. In total, sensory raters provided feedback on four samples including the control.

This study compares the R3 scramble (which had rOVD and rOVA as protein components) to the commercial products in human sensory trials. Furthermore, given their use in the market, this investigation considers a version of R3 with mung bean and chickpea (A3) replacing recombinant proteins but at the same overall protein content as R3. A3 was tested in the unseasoned category.

For the unseasoned samples, hen's egg and R3 samples were provided without salt and pepper. In this set, the experimenter included R3 and also A3. In total, sensory raters provide feedback on three unseasoned samples including the control. The mixing instructions and ingredients used are as follows:

### For A3 and R3:

TABLE 28
Scramble egg ingredients

| Ingredient | R3 | A3 |
|---|---|---|
| rOVA | 4.5% | 0.0% |
| rOVD | 1.4% | 0.0% |
| Chickpea | 0.0% | 2.5% |
| Mung bean | 0.0% | 3.4% |
| Rock salts (such as kala | 0.7% | 0.7% |
| namak) | | |
| Egg Yolk Flavor (GS) (OS) | 0.7% | 0.7% |
| Coconut oil | 1.7% | 1.7% |
| Canola oil | 2.2% | 2.2% |
| High acyl Gellan gum | 0.6% | 0.6% |
| Beta-glucan | 1.7% | 1.7% |
| Tapioca syrup DE27 | 0.5% | 0.5% |
| Pineapple yellow | 0.3% | 0.3% |
| Sunflower Lecithin | 0.7% | 0.7% |
| DI | 85% | 85% |

In addition to using an ordinal scale, this example uses the two tailed Wilcoxon-Pratt signed rank test for non-parametric paired samples. Median was used for summary statistics. For overall similarity questions, this investigation pairs the encoded similarity score for a rater in one sample with the similarity score for the same rater in another sample. For likability, this example pairs the “likability difference” score for a rater in one sample with the “likability difference” score for the same rater in a different sample.

like_{diff}=likeₛₐₘₚₗₑ−like_{control}

This in mind, this example tests the overall similarity and likability for R3 versus a commercially-available mung bean protein scramble and a commercially-available lupin protein scramble, yielding two “families” of two related tests. This investigation also examines similarity and likability against the combined mung bean/chickpea scramble (A3), yielding one “family” of two tests.

In both cases, analysis applies the Bonferroni correction.

For the other samples, the form provided the same scale of difference from control sample (“Do you like sample X?”) but also asked about similarity of the sample (“On each of these attributes, how different is sample X to the control?”) with regards to flavor, texture, appearance, and overall similarity in that order. It presents the following options for each:

TABLE 29
Difference from Control Scale

| Shown to User | Numeric Encoding |
|---|---|
| Not | 1 |
| Very slightly | 2 |
| Slight moderately | 3 |
| Moderately | 4 |
| Moderately largely | 5 |
| Largely | 6 |
| Very largely | 7 |

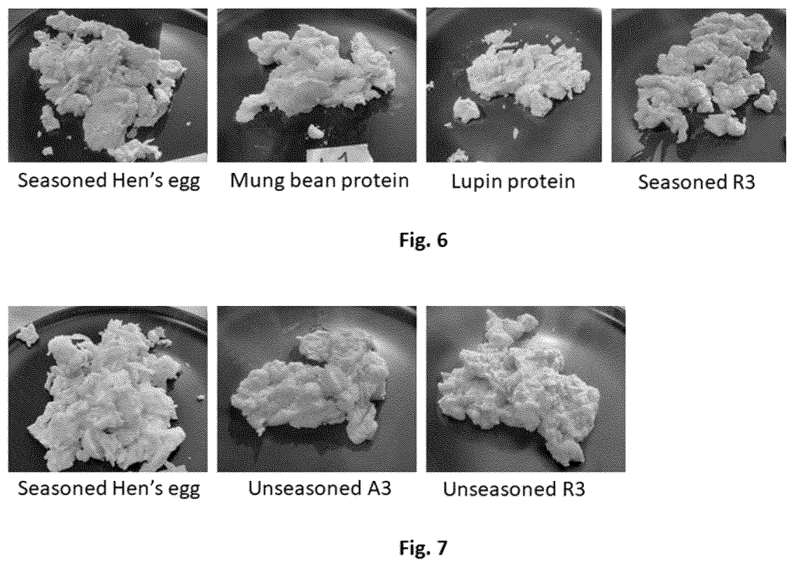

A higher likability difference means more likeable. Meanwhile, a lower overall similarity score means more similar given that the form asks in terms of degree of difference from control. In this instance, likability difference reports relative to hen's egg for ease of interpretation. However, given that both relate to the same rater's score for the same control, this means that a higher likability difference for one sample over another also means higher raw likability for that sample over the other. Results of the analysis are provided in FIGS. 6-7 and Tables 30 and 31.

TABLE 30
Likeability Results

| | Median | | p value (sample v R3) | |
|---|---|---|---|---|
| | Like | Overall | Like | Overall |
| | Diff | Similarity | Diff | Similarity |
| R3 | −1.5 | 3.0 | | |
| Commercially-available mung | −1.0 | 4.0 | 0.76 | 0.75 |
| bean protein scramble | | | | |
| Commercially-available lupin | −4.0 | 5.0 | 0.01 | 0.01 |
| protein scramble | | | | |

This study finds that the R3 scramble provides significantly better scores than a commercially-available lupin protein scramble for both overall similarity to seasoned hen's egg and likability (p<0.05/2). However, the data do not find significant difference between R3 and a commercially-available mung bean protein scramble (p≥0.05/2).

Unseasoned samples: The results in Table 31 suggest that, compared to the unseasoned A3, the unseasoned R3 scramble sees higher overall similarity to the control unseasoned hen's egg scramble and also higher likability (p<0.05/2). In other words, an unseasoned vegan scramble, replacing the recombinant egg proteins of the present disclosure with plant-based proteins results in a less desirable product. That is to say, an unexpected feature of the present disclosure is that plant-based proteins cannot replace the recombinant egg proteins when a whole-egg-like vegan scramble is desired.

TABLE 31
Similarity Results

| | likability Delta | Overall Similarity |
|---|---|---|
| R3 | −1.0 | 4.0 |
| Mung + Chickpea | −5.0 | 6.0 |
| p Value | 0.003 | 0.006 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A liquid whole egg substitute composition comprising:
(a) one or more recombinant egg-white proteins comprising at least recombinant ovalbumin (rOVA);
(b) one or more gelation agents and/or one or more thickening agents;
(c) a lipid component at a weight percent to the composition from about 2% to about 15% on a w/w or w/v basis;
(d) lecithin at a weight percent to the composition of from about 0.1% to about 1% on a w/w or w/v basis; and
(e) water,
wherein the weight percent of rOVA to the composition is at least 2% on a w/w basis.

2. The liquid whole egg substitute composition of claim 1, wherein the weight percent of rOVA to the composition is 8% to 11% on a w/w basis.

3. The liquid whole egg substitute composition of claim 1, wherein the weight percent of the lipid component to the composition is from about 2% to about 5% on a w/w or w/v basis.

4. The liquid whole egg substitute composition of claim 1, wherein the composition lacks any animal-derived substances or any animal-derived components.

5. The liquid whole egg substitute composition of claim 1, wherein when the liquid whole egg substitute composition and a whole hen's egg are prepared as a scramble, the scrambled liquid whole egg substitute composition provides sensory attributes that are comparable to those of the scrambled whole hen's egg; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

6. The liquid whole egg substitute composition of claim 1, wherein when the liquid whole egg substitute composition and a composition comprising a protein component consisting of proteins obtained from a plant are prepared as a scramble, the scrambled liquid whole egg substitute composition provides better sensory attributes than those of a scrambled composition comprising a protein component consisting of proteins obtained from a plant; wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

7. The liquid whole egg substitute composition of claim 1, wherein the rOVA is selected from the group consisting of duck OVA, ostrich OVA, quail OVA, and chicken OVA.

8. The liquid whole egg substitute composition of claim 1, wherein the glycosylation pattern of rOVA is different from a glycosylation pattern of chicken OVA.

9. The liquid whole egg substitute composition of claim 1, wherein the recombinant egg-white proteins further comprise a recombinant ovomucoid (rOVD).

10. The liquid whole egg substitute composition of claim 1, wherein the composition comprises from about 25% to about 90% water.

11. The liquid whole egg substitute composition of claim 1, wherein the amino acid profile of the recombinant egg-white proteins is closer to a whole hen's egg than the amino acid profile of a protein component consisting of proteins obtained from a plant.

12. The liquid whole egg substitute composition of claim 1, wherein the recombinant egg-white protein comprises a fraction of cysteine, methionine, and/or lysine amino acids that is closer to the fraction in a whole hen's egg than the fraction in a protein component consisting of proteins obtained from a plant.

13. The liquid whole egg substitute composition of claim 1, further comprising a coloring agent.

14. The liquid whole egg substitute composition of claim 1, wherein the composition comprises at least 1% gelation agents.

15. The liquid whole egg substitute composition of claim 1, wherein the rOVA is recombinantly produced in a yeast host cell or in a fungal host cell.

16. The liquid whole egg substitute composition of claim 15, wherein the yeast host cell is selected from a Pichia species and a Saccharomyces species, and the fungal host cell is selected from a Trichoderma species and an Aspergillus species.

17. The liquid whole egg substitute composition of claim 1, wherein the composition does not comprise any natural egg white proteins or a natural egg white.

18. The liquid whole egg substitute composition of claim 1, further comprising one or more plant proteins.

19. A liquid whole egg substitute composition comprising:
(a) recombinant egg-white proteins comprising at least recombinant ovalbumin (rOVA), wherein the rOVA is at a weight percent to the composition of at least 2% on a w/w basis;
(b) a lipid component at a weight percentage from about 2% to about 5% on a w/w or w/v basis;
(c) lecithin at a weight percent to the composition of from about 0.1% to about 1% on a w/w or w/v basis;
(d) water at a weight percent to the composition of from about 25% to about 90% on a w/w or w/v basis;
(e) a gum at a weight percent to the composition of from about 0.5% to about 2% on a w/w or w/v basis;
(f) a salt at a weight percent to the composition of from about 0.1% to about 2% on a w/w or w/v basis; and
(g) baking powder at a weight percent to the composition of from about 0.1% to about 1% on a w/w or w/v basis.

20. The liquid whole egg substitute composition of claim 19, further comprising a natural coloring or a synthetic coloring at a weight percent to the composition of from about 0.1% to about 2% on a w/w or w/v basis.

21. The liquid whole egg substitute composition of claim 19, further comprising a flavoring agent at a weight percent to the composition of from about 0.1% to about 5% on a w/w or w/v basis.

22. The liquid whole egg substitute composition of claim 19, wherein:
the amino acid profile of the recombinant egg-white proteins is closer to a whole hen's egg than the amino acid profile of a protein component consisting of proteins obtained from a plant; and/or
the nutrition value provided by amino acids of the recombinant egg-white proteins is closer to a whole hen's egg than the nutrition value provided by amino acids of a protein component consisting of proteins obtained from a plant; and/or
the recombinant egg-white protein comprises a fraction of cysteine, methionine, and/or lysine amino acids that is closer to the fraction in a whole hen's egg than the fraction in a protein component consisting of proteins obtained from a plant; and/or
the recombinant egg-white protein comprises a larger fraction of cysteine, methionine, and/or lysine amino acids than the fraction in a composition comprising a protein component consisting of proteins obtained from a plant; and/or
the recombinant egg-white protein comprises a fraction of cysteine and methionine amino acids closer to the fraction in a whole hen's egg than the fraction in a protein component consisting of proteins obtained from a plant.

23. The liquid whole egg substitute composition of claim 19,
wherein when the liquid whole egg substitute composition and a whole hen's egg are prepared as a scramble, the scrambled liquid whole egg substitute composition provides sensory attributes that are comparable to those of the scrambled whole hen's egg;
wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

24. The liquid whole egg substitute composition of claim 19, wherein when the liquid whole egg substitute composition and a composition comprising a protein component consisting of proteins obtained from a plant are prepared as a scramble, the scrambled liquid whole egg substitute composition provides better sensory attributes than those of a scrambled composition comprising a protein component consisting of proteins obtained from a plant;
wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

25. A liquid whole egg substitute composition comprising:
(a) recombinant egg-white proteins comprising at least recombinant ovalbumin (rOVA), wherein the rOVA is at a weight percent to the composition of 8% to 11% on a w/w basis;
(b) a lipid component at a weight percentage from about 2% to about 5% on a w/w or w/v basis;
(c) lecithin at a weight percent to the composition of from about 0.1% to about 1% on a w/w or w/v basis;
(d) water at a weight percent to the composition of from about 25% to about 90% on a w/w or w/v basis;
(e) a gum at a weight percent to the composition of from about 0.5% to about 1% on a w/w or w/v basis;
(f) a salt at a weight percent to the composition of from about 0.1% to about 1% on a w/w or w/v basis; and
(g) baking powder at a weight percent to the composition of from about 0.1% to about 1% on a w/w or w/v basis.

26. The liquid whole egg substitute composition of claim 25, further comprising a natural coloring or a synthetic coloring at a weight percent to the composition of from about 0.1% to about 2% on a w/w or w/v basis.

27. The liquid whole egg substitute composition of claim 25, further comprising a flavoring agent at a weight percent to the composition of from about 0.1% to about 5% on a w/w or w/v basis.

28. The liquid whole egg substitute composition of claim 25, wherein:
the amino acid profile of the recombinant egg-white proteins is closer to a whole hen's egg than the amino acid profile of a protein component consisting of proteins obtained from a plant; and/or
the nutrition value provided by amino acids of the recombinant egg-white proteins is closer to a whole hen's egg than the nutrition value provided by amino acids of a protein component consisting of proteins obtained from a plant; and/or
the recombinant egg-white protein comprises a fraction of cysteine, methionine, and/or lysine amino acids that is closer to the fraction in a whole hen's egg than the fraction in a protein component consisting of proteins obtained from a plant; and/or
the recombinant egg-white protein comprises a larger fraction of cysteine, methionine, and/or lysine amino acids than the fraction in a composition comprising a protein component consisting of proteins obtained from a plant; and/or
the recombinant egg-white protein comprises a fraction of cysteine and methionine amino acids closer to the fraction in a whole hen's egg than the fraction in a protein component consisting of proteins obtained from a plant.

29. The liquid whole egg substitute composition of claim 25,
wherein when the liquid whole egg substitute composition and a whole hen's egg are prepared as a scramble, the scrambled liquid whole egg substitute composition provides sensory attributes that are comparable to those of the scrambled whole hen's egg;
wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.

30. The liquid whole egg substitute composition of claim 25, wherein when the liquid whole egg substitute composition and a composition comprising a protein component consisting of proteins obtained from a plant are prepared as a scramble, the scrambled liquid whole egg substitute composition provides better sensory attributes than those of a scrambled composition comprising a protein component consisting of proteins obtained from a plant;
wherein the sensory attributes comprise one or more of flavor, smell, color, chewiness, texture, fluffiness, springiness, hardness, adhesiveness, fracturability, cohesiveness, gumminess, softness, graininess, mouthfeel, appearance, likeability, bite, and aftertaste.


SEQUENCE LISTING (130 sequences)

SEQ ID NO: 1 - protein, 186 aa, synthetic construct
  REGION 1..186: note = Synthetic Polypeptide
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 2 - protein, 186 aa, synthetic construct
  REGION 1..186: note = Synthentic Polypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 3 - protein, 186 aa, synthetic construct
  REGION 1..186: note = Synthentic Polypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 4 - protein, 210 aa, synthetic construct
  REGION 1..210: note = Synthentic Polypeptide
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 5 - protein, 210 aa, synthetic construct
  REGION 1..210: note = Synthentic Polypeptide
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 6 - protein, 208 aa, synthetic construct
  REGION 1..208: note = Synthentic Polypeptide
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 7 - protein, 186 aa, synthetic construct
  REGION 1..186: note = Synthentic Polypeptide
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 8 - protein, 208 aa, synthetic construct
  REGION 1..208: note = Synthentic Polypeptide
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 9 - protein, 239 aa, synthetic construct
  REGION 1..239: note = Synthentic Polypeptide
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 10 - protein, 185 aa, synthetic construct
  REGION 1..185: note = Synthentic Polypeptide
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 11 - protein, 237 aa, synthetic construct
  REGION 1..237: note = Synthentic Polypeptide
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVDCSEYPKPACTLEYRPLCGSDNKTYGNKCNFCNAVVESNGTLTLSHFGKC

SEQ ID NO: 12 - protein, 128 aa, synthetic construct
  REGION 1..128: note = Synthentic Polypeptide
EYGTNISIKHNGECKETVPMDCSRYANMTNEEGKVMMPCDRTYNPVCGTDGVTYDNECQLCAHNVEQGTSVDKKHDGVCGKELAAVSVDCSEYPKPECTAEERPICGSDNKTYGNKCNFCNAVVYVQP

SEQ ID NO: 13 - protein, 195 aa, synthetic construct
  REGION 1..195: note = Synthentic Polypeptide
VDCSRFPNTTNEEGKDVLACTKELHPICGTDGVTYSNECLLCYYNIEYGTNISKEHDGECTEAVPVDCSRYPNTTSEEGKVLIPCNRDFNPVCGSDGVTYENECLLCAHNVEQGTSVGKKHDGGCRKEFAAVSVDCSEYPKPDCTLEYRPLCGSDNKTYASKCNFCNAVVIWEQEKNTRHHASHSVFFISARLVC

SEQ ID NO: 14 - protein, 339 aa, synthetic construct
  REGION 1..339: note = Synthentic Polypeptide
MLPLGLREYGTNTSKEHDGECTEAVPVDCSRYPNTTSEEGKVRILCKKDINPVCGTDGVTYDNECLLCSHSVGQGASIDKKHDGGCRKEFAAVSVDCSEYPKPACMSEYRPLCGSDNKTYVNKCNFCNAVVYVQPWLHSRCRLPPTGTSFLGSEGRETSLLTSRATDLQVAGCTAISAMEATRAAALLGLVLLSSFCELSHLCFSQASCDVYRLSGSRNLACPRIFQPVCGTDNVTYPNECSLCRQMLRSRAVYKKHDGRCVKVDCTGYMRATGGLGTACSQQYSPLYATNGVIYSNKCTFCSAVANGEDIDLLAVKYPEEESWISVSPTPWRMLSAGA

SEQ ID NO: 15 - protein, 238 aa, synthetic construct
  REGION 1..238: note = Synthentic Polypeptide
MSWWGIKPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 16 - protein, 284 aa, synthetic construct
  REGION 1..284: note = Synthentic Polypeptide
MSSQNQLHRRRRPLPGGQDLNKYYWPHCTSDRFSWLLHVTAEQFRHCVCIYLQPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 17 - protein, 186 aa, synthetic construct
  REGION 1..186: note = Synthentic Polypeptide
VEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNVVQGTSVGKKHDGECRKELAAVSVDCSEYPKPACPKDYRPVCGSDNKTYSNKCNFCNAVVESNGTLTLNHFGKC

SEQ ID NO: 18 - protein, 210 aa, synthetic construct
  REGION 1..210: note = Synthentic Polypeptide
MAMAGVFLLFSFALCGFLPDAAFGVEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNIVQGTSVGKKHDGECRKELAAVSVDCSEYPKPACPKDYRPVCGSDNKTYSNKCNFCNAVVESNGTLTLNHFGKC

SEQ ID NO: 19 - protein, 205 aa, synthetic construct
  REGION 1..205: note = Synthentic Polypeptide
MAGVFVLLSLVLCCFPDAAFGVEVDCSRFPNTTNEEGKDVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 20 - protein, 171 aa, synthetic construct
  REGION 1..171: note = Synthentic Polypeptide
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 21 - protein, 207 aa, synthetic construct
  REGION 1..207: note = Synthentic Polypeptide
MTMPGAFVVLSFVLCCFPDATFGVEVDCSTYPNTTNEEGKEVLVCSKILSPICGTDGVTYSNECLLCANNIEYGTNISKYHDGECKEFVPVNCSRYPNTTNEEGKVMLICNKDLSPVCGTDGVTYDNECLLCAHNLEPGTSVGKKYDGECKKEIATVDCSDYPKPVCSLESMPLCGSDNKTYSNKCNFCNAVVDSNETLTLSHFGKC

SEQ ID NO: 22 - protein, 207 aa, synthetic construct
  REGION 1..207: note = Synthentic Polypeptide
MTMAGVFVLLSFALCCFPDAAFGVEVDCSTYPNTTNEEGKEVLVCTKILSPICGTDGVTYSNECLLCAYNIEYGTNVSKDHDGECKEVVPVDCSRYPNSTNEEGKVVMLCSKDLNPVCGTDGVTYDNECVLCAHNVESGTSVGKKYDGECKKETATVDCSDYPKPACTLEYMPFCGSDSKTYSNKCNFCNAVVDSNGTLTLSHFGKC

SEQ ID NO: 23 - protein, 207 aa, synthetic construct
  REGION 1..207: note = Synthentic Polypeptide
MTTAGVFVLLSFALCSFPDAAFGVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 24 - protein, 207 aa, synthetic construct
  REGION 1..207: note = Synthentic Polypeptide
MTTAGVFVLLSFTLCSFPDAAFGVEVDCSPYPNTTNEEGKEVLVCNKILSPICGTDGVTYSNECLLCAYNLEYGTNISKDYDGECKEVAPVDCSRHPNTTNEEGKVELLCNKDLNPICGTNGVTYDNECLLCARNLESGTSIGKKYDGECKKEIATVDCSDYPKPVCTLEEMPLCGSDNKTYGNKCNFCNAVVDSNGTLTLSHFGKC

SEQ ID NO: 25 - protein, 207 aa, synthetic construct
  REGION 1..207: note = Synthentic Polypeptide
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVVDSNGTLTLSRFGKC

SEQ ID NO: 26 - protein, 207 aa, synthetic construct
  REGION 1..207: note = Synthentic Polypeptide
MTMTGVFVLLSFAICCFPDAAFGVEVDCSTYPNTTNEEGKEVLVCTKILSPICGTDGVTYSNECLLCAYNIEYGTNVSKDHDGECKEVVPVDCSKYPNTTNEEGKVVLLCSKDLSPVCGTDGVTYDNECLLCARNLEPGSSVGKKYDGECKKEIATIDCSDYPKPVCSLEYMPLCGSDSKTYDNKCNFCNAVVDSNGTLTLSHFGKC

SEQ ID NO: 27 - protein, 207 aa, synthetic construct
  REGION 1..207: note = Synthentic Polypeptide
MTTAGVFVLLSFVLCCFPDAVFGVEVDCSTYPNTTNEEGKEVLVCTKILSPICGTDGVTYSNECLLCAYNIEYGTNVSKDHDGECKEVVPVNCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 28 - protein, 207 aa, synthetic construct
  REGION 1..207: note = Synthentic Polypeptide
MTTAGVFVLLSIALCCFPDAAFGVEVDCSAYSNTTSEEGKEVLSCTKILSPICGTDGVTYSNECLLCAYNIEYGTNISKDHDGECKEVVSVDCSRYPNTTNEEGKAVLLCNKDLSPVCGTDGVTYDNECLLCAHNLEPGTSVGKKYDGACKKEIATVDCSDYPKPVCTLEYLPLCGSDSKTYSNKCDFCNAVVDSNGTLTLSHFGKC

SEQ ID NO: 29 - protein, 206 aa, synthetic construct
  REGION 1..206: note = Synthentic Polypeptide
MTTAGVFVLLSFALCCFPDAAFGVEVDCSTYPNTTNEEGKEVLVCTKILSPICGTDGTTYSNECLLCAYNIEYGTNVSKDHDGECKVVPVDCSKYPNTTNEDGKVVLLCNKALSPICGTDRVTYDNECLMCAHNLEPGTSVGKKHDGECQKEVATVDCSDYPKPVCSLEYMPLCGSDGKTYSNKCNFCNAVVNSNGTLTLSHFEKC

SEQ ID NO: 30 - protein, 213 aa, synthetic construct
  REGION 1..213: note = Synthentic Polypeptide
MTTAGVFVLLSFVLCCFFPDAAFGVEVDCSTYPNTTNEEGKEVLVCAKILSPVCGTDGVTYSNECLLCAHNIENGTNVGKDHDGKCKEAVPVDCSRYPNTTDEEGKVVLLCNKDVSPVCGTDGVTYDNECLLCAHNLEAGTSVDKKNDSECKTEDTTLAAVSVDCSDYPKPVCTLEYLPLCGSDNKTYSNKCRFCNAVVDSNGTLTLSRFGKC

SEQ ID NO: 31 - protein, 207 aa, synthetic construct
  REGION 1..207: note = Synthentic Polypeptide
MTTAGVFVLLSFALCCSPDAAFGVEVDCSTYPNTTNEEGKEVLACTKILSPICGTDGVTYSNECLLCAYNMEYGTNVSKDHDGKCKEVVPVDCSRYPNTTNEEGKVVLLCNKDLSPVCGTDGVTYDNECLLCARNLEPGASVGKKYDGECKKEIATVDCSDYPKPVCSLEHMPLCGSDSKTYSNKCTFCNAVVDSNGTLTLSHFGKC

SEQ ID NO: 32 - protein, 207 aa, synthetic construct
  REGION 1..207: note = Synthentic Polypeptide
MTTAGVFVLLSFALCCFPDAAFGVEVDCSTYPNTTNEEGREVLVCTKILSPICGTDGVTYSNECLLCAYNIEYGTNVSKDHDGECKEVAPVGCSRYPNTTNEEGKVVLLCNKDLSPVCGTDGVTYDNECLLCARHLEPGTSVGKKYDGECKKEIATVDCSDYPKPVCSLEYMPLCGSDSKTYSNKCNFCNAVLDSNGTLTLSHFGKC

SEQ ID NO: 33 - protein, 207 aa, synthetic construct
  REGION 1..207: note = Synthentic Polypeptide
MTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 34 - protein, 207 aa, synthetic construct
  REGION 1..207: note = Synthentic Polypeptide
MTTAGVFVLLSFVLCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 35 - protein, 282 aa, synthetic construct
  REGION 1..282: note = Synthentic Polypeptide
MSSQNQLPSRCRPLPGSQDLNKYYQPHCTGDRFCWLFYVTVEQFRHCICIYLQLALERPSHEQSGQPADSRNTSTMTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 36 - protein, 193 aa, synthetic construct
  REGION 1..193: note = Synthentic Polypeptide
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVV

SEQ ID NO: 37 - protein, 190 aa, synthetic construct
  REGION 1..190: note = Synthentic Polypeptide
EAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 38 - protein, 199 aa, synthetic construct
  REGION 1..199: note = Synthentic Polypeptide
EEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 39 - protein, 275 aa, synthetic construct
  REGION 1..275: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 40 - protein, 273 aa, synthetic construct
  REGION 1..273: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 41 - protein, 188 aa, synthetic construct
  REGION 1..188: note = Synthentic Polypeptide
EAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 42 - protein, 206 aa, synthetic construct
  REGION 1..206: note = Synthentic Polypeptide
MTMAGVFVLLSFILCCFPDTAFGVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 43 - protein, 272 aa, synthetic construct
  REGION 1..272: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 44 - protein, 187 aa, synthetic construct
  REGION 1..187: note = Synthentic Polypeptide
EAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 45 - protein, 474 aa, synthetic construct
  REGION 1..474: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 46 - protein, 389 aa, synthetic construct
  REGION 1..389: note = Synthentic Polypeptide
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 47 - protein, 419 aa, synthetic construct
  REGION 1..419: note = Synthentic Polypeptide
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 48 - protein, 406 aa, synthetic construct
  REGION 1..406: note = Synthentic Polypeptide
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 49 - protein, 451 aa, synthetic construct
  REGION 1..451: note = Synthentic Polypeptide
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 50 - protein, 386 aa, synthetic construct
  REGION 1..386: note = Synthentic Polypeptide
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 51 - protein, 386 aa, synthetic construct
  REGION 1..386: note = Synthentic Polypeptide
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 52 - protein, 417 aa, synthetic construct
  REGION 1..417: note = Synthentic Polypeptide
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 53 - protein, 383 aa, synthetic construct
  REGION 1..383: note = Synthentic Polypeptide
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 54 - protein, 386 aa, synthetic construct
  REGION 1..386: note = Synthentic Polypeptide
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 55 - protein, 411 aa, synthetic construct
  REGION 1..411: note = Synthentic Polypeptide
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 56 - protein, 383 aa, synthetic construct
  REGION 1..383: note = Synthentic Polypeptide
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 57 - protein, 408 aa, synthetic construct
  REGION 1..408: note = Synthentic Polypeptide
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 58 - protein, 383 aa, synthetic construct
  REGION 1..383: note = Synthentic Polypeptide
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 59 - protein, 386 aa, synthetic construct
  REGION 1..386: note = Synthentic Polypeptide
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 60 - protein, 386 aa, synthetic construct
  REGION 1..386: note = Synthentic Polypeptide
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 61 - protein, 386 aa, synthetic construct
  REGION 1..386: note = Synthentic Polypeptide
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 62 - protein, 386 aa, synthetic construct
  REGION 1..386: note = Synthentic Polypeptide
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 63 - protein, 386 aa, synthetic construct
  REGION 1..386: note = Synthentic Polypeptide
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 64 - protein, 386 aa, synthetic construct
  REGION 1..386: note = Synthentic Polypeptide
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 65 - protein, 386 aa, synthetic construct
  REGION 1..386: note = Synthentic Polypeptide
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 66 - protein, 386 aa, synthetic construct
  REGION 1..386: note = Synthentic Polypeptide
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 67 - protein, 386 aa, synthetic construct
  REGION 1..386: note = Synthentic Polypeptide
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 68 - protein, 386 aa, synthetic construct
  REGION 1..386: note = Synthentic Polypeptide
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 69 - protein, 386 aa, synthetic construct
  REGION 1..386: note = Synthentic Polypeptide
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 70 - protein, 386 aa, synthetic construct
  REGION 1..386: note = Synthentic Polypeptide
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 71 - protein, 391 aa, synthetic construct
  REGION 1..391: note = Synthentic Polypeptide
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 72 - protein, 386 aa, synthetic construct
  REGION 1..386: note = Synthentic Polypeptide
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 73 - protein, 386 aa, synthetic construct
  REGION 1..386: note = Synthentic Polypeptide
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 74 - protein, 386 aa, synthetic construct
  REGION 1..386: note = Synthentic Polypeptide
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 75 - protein, 386 aa, synthetic construct
  REGION 1..386: note = Synthentic Polypeptide
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 76 - protein, 385 aa, synthetic construct
  REGION 1..385: note = Synthentic Polypeptide
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 77 - protein, 386 aa, synthetic construct
  REGION 1..386: note = Synthentic Polypeptide
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 78 - protein, 386 aa, synthetic construct
  REGION 1..386: note = Synthentic Polypeptide
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 79 - protein, 386 aa, synthetic construct
  REGION 1..386: note = Synthentic Polypeptide
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 80 - protein, 385 aa, synthetic construct
  REGION 1..385: note = Synthentic Polypeptide
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 81 - protein, 386 aa, synthetic construct
  REGION 1..386: note = Synthentic Polypeptide
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 82 - protein, 386 aa, synthetic construct
  REGION 1..386: note = Synthentic Polypeptide
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 83 - protein, 386 aa, synthetic construct
  REGION 1..386: note = Synthentic Polypeptide
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 84 - protein, 386 aa, synthetic construct
  REGION 1..386: note = Synthentic Polypeptide
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 85 - protein, 386 aa, synthetic construct
  REGION 1..386: note = Synthentic Polypeptide
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 86 - protein, 386 aa, synthetic construct
  REGION 1..386: note = Synthentic Polypeptide
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 87 - protein, 386 aa, synthetic construct
  REGION 1..386: note = Synthentic Polypeptide
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 88 - protein, 386 aa, synthetic construct
  REGION 1..386: note = Synthentic Polypeptide
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 89 - protein, 386 aa, synthetic construct
  REGION 1..386: note = Synthentic Polypeptide
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 90 - protein, 381 aa, synthetic construct
  REGION 1..381: note = Synthentic Polypeptide
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 91 - protein, 391 aa, synthetic construct
  REGION 1..391: note = Synthentic Polypeptide
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 92 - protein, 383 aa, synthetic construct
  REGION 1..383: note = Synthentic Polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 93 - protein, 386 aa, synthetic construct
  REGION 1..386: note = Synthentic Polypeptide
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 94 - protein, 392 aa, synthetic construct
  REGION 1..392: note = Synthentic Polypeptide
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 95 - protein, 386 aa, synthetic construct
  REGION 1..386: note = Synthentic Polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 96 - protein, 395 aa, synthetic construct
  REGION 1..395: note = Synthentic Polypeptide
  SITE 99: note = misc_feature - Xaa can be any naturally occurring amino acid
  SITE 233: note = misc_feature - Xaa can be any naturally occurring amino acid
  SITE 341: note = misc_feature - Xaa can be any naturally occurring amino acid
  SITE 379: note = misc_feature - Xaa can be any naturally occurring amino acid
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 97 - protein, 386 aa, synthetic construct
  REGION 1..386: note = Synthentic Polypeptide
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 98 - protein, 386 aa, synthetic construct
  REGION 1..386: note = Synthentic Polypeptide
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 99 - protein, 385 aa, synthetic construct
  REGION 1..385: note = Synthentic Polypeptide
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 100 - protein, 386 aa, synthetic construct
  REGION 1..386: note = Synthentic Polypeptide
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 101 - protein, 383 aa, synthetic construct
  REGION 1..383: note = Synthentic Polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 102 - protein, 386 aa, synthetic construct
  REGION 1..386: note = Synthentic Polypeptide
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 103 - protein, 386 aa, synthetic construct
  REGION 1..386: note = Synthentic Polypeptide
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 104 - protein, 2174 aa, synthetic construct
  REGION 1..2174: note = Synthentic Polypeptide
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 105 - protein, 386 aa, synthetic construct
  REGION 1..386: note = Synthentic Polypeptide
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 106 - protein, 2104 aa, synthetic construct
  REGION 1..2104: note = Synthentic Polypeptide
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 107 - protein, 386 aa, synthetic construct
  REGION 1..386: note = Synthentic Polypeptide
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 108 - protein, 420 aa, synthetic construct
  REGION 1..420: note = Synthentic Polypeptide
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 109 - protein, 386 aa, synthetic construct
  REGION 1..386: note = Synthentic Polypeptide
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 110 - protein, 386 aa, synthetic construct
  REGION 1..386: note = Synthentic Polypeptide
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 111 - protein, 386 aa, synthetic construct
  REGION 1..386: note = Synthentic Polypeptide
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 112 - protein, 386 aa, synthetic construct
  REGION 1..386: note = Synthentic Polypeptide
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 113 - protein, 386 aa, synthetic construct
  REGION 1..386: note = Synthentic Polypeptide
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 114 - protein, 399 aa, synthetic construct
  REGION 1..399: note = Synthentic Polypeptide
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 115 - protein, 389 aa, synthetic construct
  REGION 1..389: note = Synthentic Polypeptide
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 116 - protein, 474 aa, synthetic construct
  REGION 1..474: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 117 - protein, 389 aa, synthetic construct
  REGION 1..389: note = Synthentic Polypeptide
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 118 - protein, 474 aa, synthetic construct
  REGION 1..474: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 119 - protein, 218 aa, synthetic construct
  REGION 1..218: note = Synthentic Polypeptide
MRFPSIFTAVLFAASSALAAPVNTTTEDETAQIPAEAVIGYSDLEGDFDVAVLPFSNSTNNGLLFINTTIASIAAKEEGVSLDKREAEAKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 120 - protein, 133 aa, synthetic construct
  REGION 1..133: note = Synthentic Polypeptide
EAEAKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 121 - protein, 129 aa, synthetic construct [sequence omitted]

SEQ ID NO: 122 - protein, 129 aa, synthetic construct
  REGION 1..129: note = Synthentic Polypeptide
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 123 - protein, 130 aa, synthetic construct
  REGION 1..130: note = Synthentic Polypeptide
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 124 - protein, 129 aa, synthetic construct
  REGION 1..129: note = Synthentic Polypeptide
KVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 125 - protein, 147 aa, synthetic construct
  REGION 1..147: note = Synthentic Polypeptide
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 126 - protein, 185 aa, synthetic construct [sequence omitted]

SEQ ID NO: 127 - protein, 184 aa, synthetic construct
  REGION 1..184: note = Synthentic Polypeptide
SGCYGNIMDVPTTGASCLTASQDNLPYCGVAASQQMAATDLPDMNQYKEKILAVAQNLCMDGAVIAGIISRESRAGAVLQNGWGDNGHAFGLMQIDIRWHSIEGAWNSQENINEGTGILINMIVAISDKFPSWSVNDNLKGGIAAYNAGPGNIYSYSQVDQYTTDGDYSNDVVARAQYYKTQGY

SEQ ID NO: 128 - protein, 266 aa, synthetic construct
  REGION 1..266: note = Synthentic Polypeptide
FRYAILAREEEPRVRRAALVDKPRVEIADVLISTFTESGVIEVVLQALREIGCNDLRERFAKDTSEGSPTSASKYGDIMKVETTGASMQTAQQDYLDFSGARASHAMAETDLIEMNNYKSVIKNAAGKKGVDPALIAAMISRSCRAGKTLSGGWGCWDEKRQKYNTYGLMQIDVNPKGGGHTPKGSWDSEEHLCQAIDILIRFITRIRQKYPQWSKEEQLKGGIAAYNAGDGNIGPGKDVDSKTTNGDYANDIVARAQWYKSNGGF

SEQ ID NO: 129 - protein, 120 aa, synthetic construct
  REGION 1..120: note = Synthentic Polypeptide
AHNFATGIVPQSCLECICKTESGCRAIGCKFDVYSDSCGYFQLKQAYWEDCGRPGGSLTSCADDIHCSSQCVQHYMSRYIGHTSCSRTCESYARLHNGGPHGCEHGSTLGYWGHVQGHGC

SEQ ID NO: 130 - protein, 4 aa, synthetic construct
  REGION 1..4: note = Synthetic peptide
EAEA